(19) United States
(12) Patent Application Publication
(10) Pub. No.: US 2023/0141988 A1
(43) Pub. Date: May 11, 2023

(54) PHARMACEUTICAL COMPOSITION FOR USE IN THE TREATMENT OF NEUROLOGICAL DISEASES

(71) Applicant: Aclipse One Inc., Radnor, PA (US)

(72) Inventor: Ning Shan, Chandler, AZ (US)

(21) Appl. No.: 17/633,150

(22) PCT Filed: Aug. 7, 2020
(86) PCT No.: PCT/US2020/045321

§ 371 Date: Feb. 4, 2022

(51) Int. Cl. A61K 31/473; A61K 9/00

(52) U.S. Cl. CPC A61K 31/473; A61K 9/0056; A61K 9/0019; A61K 9/0043; A61K 9/006

(57) ABSTRACT

The present invention relates to pharmaceutical compositions for administration to mammals that include (6aS)-6-methyl-5,6,6a,7-tetrahydro-4H-dibenzo[de,g]quinoline-10,11-diol or a pharmaceutically acceptable salt thereof and at least one pharmaceutically acceptable excipient that provides pharmacokinetic profiles useful for the treatment of neurodegenerative diseases.

### CROSS-REFERENCE TO RELATED APPLICATION

This application claims the benefit of U.S. Provisional Application No. 62/883,903, filed Aug. 7, 2019, the disclosure of which is hereby incorporated herein by reference in its entirety.

### FIELD OF INVENTION

This invention relates to pharmaceutical compositions comprising (6aS)-6-methyl-5,6,6a,7-tetrahydro-4H-dibenzo[de,g]quinoline-10,11-diol and salt forms thereof useful for the treatment of neurological diseases, processes for preparing such formulations, and pharmacokinetic parameters controlled by the same.

### BACKGROUND

Pharmacokinetics is a subdivision of pharmacology which addresses the concentrations or quantities of drug substances and their metabolites in biological fluids, tissues and excreta as a function of time. In general, pharmacokinetic studies focus upon the understanding and characterization of drug absorption, distribution, metabolism and excretion, as well as their relationships to pharmacodynamics and toxicology. Particularly, drug absorption, the transport of active pharmaceutical ingredients (APIs) into blood circulation, represents a critical PK process for orally administered drugs. Most APIs are solids under ambient conditions and most drug products are developed and orally delivered as solid dosage forms. The oral absorption of a drug is typically a two-step process. Firstly, the drug product dissolves into the biological fluids secreted in the gastrointestinal (GI) tract. This releases API molecules that can then permeate across the GI membrane via modes such as passive diffusion or active transport. Solubility and permeability are therefore two critical parameters that significantly impact the efficiency of oral drug absorption. In this context, the United States Food and Drug Administration (FDA) issued a Guidance for Industry to address the need to identify and characterize solid forms of drug molecules and classify orally administrated drugs, via the Biopharmaceutics Classification System (BCS), according to their solubility and permeability. With respect to BCS, a drug molecule is considered highly soluble when the highest dose strength dissolves in less than 250 ml of water over a pH range from 1 to 7.5. Also, a drug molecule is considered highly permeable when the extent of absorption in humans is determined to be more than 90% of the administered dose based on mass-balance or in comparison to an intravenous reference dose. It has been noted that ca. 30% of the orally administered drugs currently on the market belong to BCS class II (i.e., low solubility and high permeability). Moreover, approximately 70% of the new chemical entities (NCEs) under development, potentially for oral administration, also exhibit low solubilities. In drug development, much effort has been directed towards improving the aqueous solubility of poorly soluble APIs.

(6aS)-6-methyl-5,6,6a,7-tetrahydro-4H-dibenzo[de,g]quinoline-10,11-diol is a weak dopamine antagonist for the treatment of neurological disorders. (6aS)-6-methyl-5,6,6a,7-tetrahydro-4H-dibenzo[de,g]quinoline-10,11-diol, also known as S-(+)-10,11-dihydroxyaporphine, is depicted by the following chemical structure:

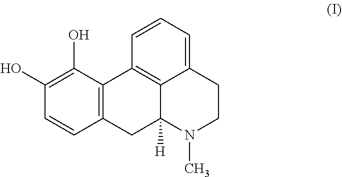 [SMILES: CN1CCc2cccc3c2[C@@H]1Cc1ccc(O)c(O)c1-3]

Free base of (6aS)-6-methyl-5,6,6a,7-tetrahydro-4H-dibenzo[de,g]quinoline-10,11-diol exhibited low solubilities in the aqueous media at various pH values. The hydrochloride salt of (6aS)-6-methyl-5,6,6a,7-tetrahydro-4H-dibenzo[de,g]quinoline-10,11-diol also showed low solubility in the aqueous buffer solutions. Moreover, (6aS)-6-methyl-5,6,6a,7-tetrahydro-4H-dibenzo[de,g]quinoline-10,11-diol undertook significant oxidation in the liquid solutions, indicating a high level of decomposition in the GI tract and through first-pass metabolism. Therefore, systemic exposure of (6aS)-6-methyl-5,6,6a,7-tetrahydro-4H-dibenzo[de,g]quinoline-10,11-diol after its oral administration remains as a challenge. Compared to parenteral delivery, it was also note that (6aS)-6-methyl-5,6,6a,7-tetrahydro-4H-dibenzo[de,g]quinoline-10,11-diol is expected to exhibit a lower Cₘₐₓ (maximal drug concentration) after oral administration. Knowing that most APIs cross the blood-brain barrier (BBB) via passive diffusion, a lower Cₘₐₓ could lead to a poor transport of API molecules to the brain. Consequently, the exposure of (6aS)-6-methyl-5,6,6a,7-tetrahydro-4H-dibenzo[de,g]quinoline-10,11-diol in the central nervous system (CNS) via oral delivery is also problematic.

### BRIEF DESCRIPTION OF THE DRAWINGS

FIG. 1. Plasma Concentration-Time Profiles of (6aS)-6-methyl-5,6,6a,7-tetrahydro-4H-dibenzo[de,g]quinoline-10,11-diol after subcutaneous administration at 4.40 mg/kg to male C57BL/6J mice (n=3).

FIG. 2. Plasma Concentration-Time Profiles of (6aR)-6-methyl-5,6,6a,7-tetrahydro-4H-dibenzo[de,g]quinoline-10,11-diol after subcutaneous administration at 0.0367 mg/kg to male C57BL/6J mice (n=3).

FIG. 3. Plasma Concentration-Time Profiles of (6aS)-6-methyl-5,6,6a,7-tetrahydro-4H-dibenzo[de,g]quinoline-10,11-diol after subcutaneous administration to male C57BL/6J Mice (mean±SD, n=3).

FIG. 4. Plasma Concentration-Time Profiles of (6aR)-6-methyl-5,6,6a,7-tetrahydro-4H-dibenzo[de,g]quinoline-10,11-diol after subcutaneous administration to male C57BL/6J Mice (mean±SD, n=3).

FIG. 5. Plasma Concentration-Time Profiles of (6aS)-6-methyl-5,6,6a,7-tetrahydro-4H-dibenzo[de,g]quinoline-10,11-diol after intravenous (IV) Administration at 0.880 mg/kg to male SD Rats (n=3).

FIG. 6. Plasma Concentration-Time Profiles of (6aS)-6-methyl-5,6,6a,7-tetrahydro-4H-dibenzo[de,g]quinoline-10,11-diol after subcutaneous administration at 0.880 mg/kg to male SD Rats (n=3).

FIG. 7. Plasma Concentration-Time Profiles of (6aS)-6-methyl-5,6,6a,7-tetrahydro-4H-dibenzo[de,g]quinoline-10,11-diol after oral (PO) Administration at 4.40 mg/kg to male SD Rats (n=3).

FIG. 8. Plasma Concentration-Time Profiles of (6aS)-6-methyl-5,6,6a,7-tetrahydro-4H-dibenzo[de,g]quinoline-10,11-diol after intranasal (IN) Administration at 1.76 mg/kg to male SD Rats (n=3).

FIG. 9. Plasma Concentration-Time Profiles of (6aS)-6-methyl-5,6,6a,7-tetrahydro-4H-dibenzo[de,g]quinoline-10,11-diol after IV, subcutaneous, PO and IN administration to male SD Rats (mean±SD, n=3).

FIG. 10. Plasma Concentration-Time Profiles of (6aR)-6-methyl-5,6,6a,7-tetrahydro-4H-dibenzo[de,g]quinoline-10,11-diol after IV Administration at 0.880 mg/kg to male SD Rats (n=3).

FIG. 11. Individual and mean plasma concentration profiles of (6aS)-6-methyl-5,6,6a,7-tetrahydro-4H-dibenzo[de,g]quinoline-10,11-diol in male Beagle dogs following single sublingual (SL) administration of (6aS)-6-methyl-5,6,6a,7-tetrahydro-4H-dibenzo[de,g]quinoline-10,11-diol at 5 mg/kg.

FIG. 12. Individual and mean CSF concentration profiles of (6aS)-6-methyl-5,6,6a,7-tetrahydro-4H-dibenzo[de,g]quinoline-10,11-diol in male Beagle dogs following single SL administration of (6aS)-6-methyl-5,6,6a,7-tetrahydro-4H-dibenzo[de,g]quinoline-10,11-diol at 5 mg/kg.

FIG. 13. Mean plasma and CSF concentration profiles of (6aS)-6-methyl-5,6,6a,7-tetrahydro-4H-dibenzo[de,g]quinoline-10,11-diol in male Beagle dogs following single SL administration of (6aS)-6-methyl-5,6,6a,7-tetrahydro-4H-dibenzo[de,g]quinoline-10,11-diol at 5 mg/kg.

FIG. 14. Individual and mean plasma concentration profiles of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg.

FIG. 15. Individual and mean CSF concentration profiles of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg.

FIG. 16. Mean plasma and CSF concentration profiles of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg.

FIG. 17. Individual and mean plasma concentration profiles of (6aS)-6-methyl-5,6,6a,7-tetrahydro-4H-dibenzo[de,g]quinoline-10,11-diol in male Beagle dogs following single SC administration of (6aS)-6-methyl-5,6,6a,7-tetrahydro-4H-dibenzo[de,g]quinoline-10,11-diol at 1 mg/kg.

FIG. 18. Individual and mean CSF concentration profiles of (6aS)-6-methyl-5,6,6a,7-tetrahydro-4H-dibenzo[de,g]quinoline-10,11-diol in male Beagle dogs following single SC administration of (6aS)-6-methyl-5,6,6a,7-tetrahydro-4H-dibenzo[de,g]quinoline-10,11-diol at 1 mg/kg.

FIG. 19. Mean plasma and CSF concentration profiles of (6aS)-6-methyl-5,6,6a,7-tetrahydro-4H-dibenzo[de,g]quinoline-10,11-diol in male Beagle dogs following single SC administration of (6aS)-6-methyl-5,6,6a,7-tetrahydro-4H-dibenzo[de,g]quinoline-10,11-diol at 1 mg/kg.

FIG. 20. Mean plasma concentration profiles of (6aS)-6-methyl-5,6,6a,7-tetrahydro-4H-dibenzo[de,g]quinoline-10,11-diol in male Beagle dogs following single SL, PO and SC administration of (6aS)-6-methyl-5,6,6a,7-tetrahydro-4H-dibenzo[de,g]quinoline-10,11-diol at 5, 10 and 1 mg/kg, respectively.

FIG. 21. Mean CSF concentration profiles of (6aS)-6-methyl-5,6,6a,7-tetrahydro-4H-dibenzo[de,g]quinoline-10,11-diol in male Beagle dogs following single SL, PO and SC administration of (6aS)-6-methyl-5,6,6a,7-tetrahydro-4H-dibenzo[de,g]quinoline-10,11-diol at 5, 10 and 1 mg/kg, respectively.

FIG. 22. Individual and mean plasma concentration profiles of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg.

FIG. 23. Individual and mean CSF concentration profiles of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg.

FIG. 24. Mean plasma and SCF concentration profiles of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg.

FIG. 25. Plasma concentration versus time profile after 1.14 mg/kg SC administration of (6aS)-6-methyl-5,6,6a,7-tetrahydro-4H-dibenzo[de,g]quinoline-10,11-diol in Cynomolgus Monkey.

### DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a,” “an,” and “the” include plural reference unless the context clearly dictates otherwise. Thus, for example, reference to a “cell” is a reference to one or more cells and equivalents thereof known to those skilled in the art, and so forth.

As used herein, the term “about” means deviation of up to 20% of the numerical value of the number with which it is being used. Therefore, about 50% means in the range of 40%-60%.

“Administering” when used in conjunction with a therapeutic means to administer a therapeutic directly to a subject, whereby the agent positively impacts the target. “Administering” a composition may be accomplished by, for example, injection, oral administration, topical administration, or by these methods in combination with other known techniques. Such combination techniques include heating, radiation, ultrasound and the use of delivery agents. When a compound is provided in combination with one or more other active agents (e.g. other anti-atherosclerotic agents such as the class of statins), “administration” and its variants are each understood to include concurrent and sequential provision of the compound or salt and other agents.

By “pharmaceutically acceptable” it is meant the carrier, diluent, adjuvant, or excipient must be compatible with the other ingredients of the formulation and not deleterious to the recipient thereof.

“Composition” as used herein is intended to encompass a product comprising the specified ingredients in the specified amounts, as well as any product which results, directly or indirectly, from combination of the specified ingredients in the specified amounts. Such term in relation to “pharmaceutical composition” is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound o the present invention and a pharmaceutically acceptable carrier.

As used herein, the term “agent,” “active agent,” “therapeutic agent,” or “therapeutic” means a compound or composition utilized to treat, combat, ameliorate, prevent or improve an unwanted condition or disease of a patient. Furthermore, the term “agent,” “active agent,” “therapeutic agent,” or “therapeutic” encompasses a combination of one or more of the compounds of the present invention.

A “therapeutically effective amount” or “effective amount” of a composition is a predetermined amount calculated to achieve the desired effect, i.e., to inhibit, block, or reverse the activation, migration, proliferation, alteration of cellular function, and to preserve the normal function of cells. The activity contemplated by the methods described herein includes both medical therapeutic and/or prophylactic treatment, as appropriate, and the compositions of the invention may be used to provide improvement in any of the conditions described. It is also contemplated that the compositions described herein may be administered to healthy subjects or individuals not exhibiting symptoms but who may be at risk of developing a particular disorder. The specific dose of a compound administered according to this invention to obtain therapeutic and/or prophylactic effects will, of course, be determined by the particular circumstances surrounding the case, including, for example, the compound administered, the route of administration, and the condition being treated. However, it will be understood that the chosen dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refer to both therapeutic treatment and prophylactic or preventative measures, wherein the object is to prevent or slow down (lessen) an undesired physiological condition, disorder, or disease, or to obtain beneficial or desired clinical results. For the purposes of this invention, beneficial or desired results include, but are not limited to, alleviation of symptoms; diminishment of the extent of the condition, disorder, or disease; stabilization (i.e., not worsening) of the state of the condition, disorder, or disease; delay in onset or slowing of the progression of the condition, disorder, or disease; amelioration of the condition, disorder, or disease state; and remission (whether partial or total), whether detectable or undetectable, or enhancement or improvement of the condition, disorder, or disease. Treatment includes prolonging survival as compared to expected survival if not receiving treatment.

The term “AUC” refers to the area under the plasma concentration-time curve. “AUCₗₐₛₜ” refers to the area under the plasma concentration-time curve from zero to the last quantifiable concentration time. The term “AUCₐₗₗ” refers to the area under the plasma concentration-time curve from the time of dosing to the time of the last observation, regardless of whether the last concentration is measureable or not. The term “AUC_{inf}” refers to the area under the plasma concentration-time curve from time zero extrapolated to infinity. The term “Cₘₐₓ” refers to maximum concentration. The term “C₀” refers to initial or back-extrapolated drug concentration at time zero following bolus intravenous injection, and the term “Tₘₐₓ” refers to the time when Cₘₐₓ is achieved. The term “t_{1/2}” refers to the half-life, which is the period of time required for the concentration or amount of drug in the body to be reduced by one-half. The term “(6aS)-6-methyl-5,6,6a,7-tetrahydro-4H-dibenzo[de,g]quinoline-10,11-diol” or a pharmaceutically acceptable salt thereof may also be referred to as “Compound 1”.

The term “bioavailability” or “F %” refers to the rate and extent to which (6aS)-6-methyl-5,6,6a,7-tetrahydro-4H-dibenzo[de,g]quinoline-10,11-diol is absorbed into a biological system from an administered drug product and becomes available at the site of biological action. The values for bioavailability may be represented in relative terms. For example, oral bioavailability may be represented in terms of percentages relative to intravenous administration or subcutaneous administration.

Administration and Compositions

The pharmaceutical compositions included within the scope of the present invention comprise a therapeutically effective amount Compound 1 and at least one pharmaceutically acceptable excipient. The term “excipient” refers to a pharmaceutically acceptable, inactive substance used as a carrier for the pharmaceutically active ingredient (Compound 1), and includes anti adherents, binders, coatings, disintegrants, fillers, diluents, solvents, flavors, bulkants, colours, glidants, dispersing agents, wetting agents, lubricants, preservatives, sorbents and sweeteners. The choice of excipient(s) will depend on factors such as the particular mode of administration and the nature of the dosage form. Solutions or suspensions used for injection or infusion can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, including autoinjectors, or multiple dose vials made of glass or plastic.

In the methods of various embodiments, pharmaceutical compositions including the active agent can be administered to a subject in an “effective amount” or “therapeutically effective amount,” which may be any amount that provides a beneficial effect to the subject.

A pharmaceutical formulation of the present invention may be in any pharmaceutical dosage form. The pharmaceutical formulation may be, for example, a tablet, capsule, nanoparticulate material, e.g., granulated particulate material or a powder, a lyophilized material for reconstitution, liquid solution, suspension, emulsion or other liquid form, injectable suspension, solution, emulsion, etc., suppository, or topical or transdermal preparation or patch. The pharmaceutical formulations generally contain about 1% to about 99% by weight of Compound 1 and 99% to 1% by weight of a suitable pharmaceutical excipient. In one embodiment, the dosage form is an oral dosage form. In another embodiment, the dosage form is a parenteral dosage form. In another embodiment, the dosage form is an enteral dosage form. In another embodiment, the dosage form is a topical dosage form. In one embodiment, the pharmaceutical dosage form is a unit dose. The term ‘unit dose’ refers to the amount of Compound 1 administered to a patient in a single dose.

A pharmaceutical formulation may be, for example, an oral dosage form for controlled release. By way of example only, controlled or modified release oral dosage forms can be prepared by using methods known in the art. For example, a suitable controlled release form of Compound I may be a matrix tablet or a capsule dosage composition. Suitable materials for matrix dosage forms include, for example, waxes (e.g. carnauba, bees wax, paraffin wax, ceresine, shellac wax, fatty acids, and fatty alcohols), oils, hardened oils or fats (e.g., hardened rapeseed oil, castor oil, beef tallow palm oil, and soya bean oil), and polymers (e.g., hydroxypropyl cellulose, polyvinylpyrrolidone, hydroxypropyl methyl cellulose, and polyethylene glycol). Other suitable matrix tableting materials include microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. The finished tablet may also be coated or uncoated.

The coating composition typically contains an insoluble matrix polymer (approximately 15-85% by weight of the coating composition) and a water soluble material (e.g., approximately 15-85% by weight of the coating composition). Optionally an enteric polymer (approximately 1 to 99% by weight of the coating composition) may be used or included. Suitable water soluble materials include polymers such as polyethylene glycol, hydroxypropyl cellulose, hydroxypropyl methyl cellulose, polyvinylpyrrolidone, polyvinyl alcohol, and monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups. The coating composition may be plasticised according to the properties of the coating blend such as the glass transition temperature of the main component or mixture of components or the solvent used for applying the coating compositions. Suitable plasticisers may be added from 0 to 50% by weight of the coating composition and include, for example, diethyl phthalate, citrate esters, polyethylene glycol, glycerol, acetylated glycerides, acetylated citrate esters, dibutylsebacate, and castor oil. If desired, the coating composition may include a filler. The amount of the filler may be 1% to approximately 99% by weight based on the total weight of the coating composition and may be an insoluble material such as silicon dioxide, titanium dioxide, talc, kaolin, alumina, starch, powdered cellulose, MCC, or polacrilin potassium. The coating composition may be applied as a solution or latex in organic solvents or aqueous solvents or mixtures thereof. If solutions are applied, the solvent may be present in amounts from approximate by 25-99% by weight based on the total weight of dissolved solids. Suitable solvents are water, lower alcohol, lower chlorinated hydrocarbons, ketones, or mixtures thereof. If latexes are applied, the solvent is present in amounts from approximately 25-97% by weight based on the quantity of polymeric material in the latex. The solvent may be predominantly water.

In some embodiments, a pharmaceutical composition of the present invention is delivered to a subject via a parenteral route, an enteral route, or a topical route. Examples of parental routes the present invention include, without limitation, any one or more of the following: intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumoral, intratympanic, intrauterine, intravascular, intravenous (bolus or drip), intraventricular, intravesical, and/or subcutaneous.

Enteral routes of administration of the present invention include administration to the gastrointestinal tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories. Oral administration includes sublingual and buccal administration.

Topical administration includes administration to a body surface, such as skin or mucous membranes, including intranasal and pulmonary administration. Transdermal forms include cream, foam, gel, lotion or ointment. Intranasal and pulmonary forms include liquids and powders, e.g., liquid spray.

The dose may vary depending upon the dosage form employed, sensitivity of the patient, and the route of administration. Dosage and administration are adjusted to provide sufficient levels of the active agent(s) or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy.

In one embodiment, the daily dose of Compound 1 administered to a patient is selected from: up to 200 mg, 175 mg, 150 mg, 125 mg, 100 mg, 90 mg, 80 mg, 70 mg, 60 mg, 50 mg, 30 mg, 25 mg, 20 mg, 15 mg, 14 mg, 13 mg, 12 mg, 11 mg, 10 mg, 9 mg, 8 mg, 7 mg, 6 mg, 5 mg, 4 mg, 3 mg, 2 mg, 1 mg, 0.5 mg, or up to 0.1 mg.

In another embodiment, the daily dose is at least 0.05 mg, 0.1 mg, 0.5 mg, 1 mg, 2 mg, 3 mg, 4 mg, 5 mg, 6 mg, 7 mg, 8 mg, 9 mg, 10 mg, 12 mg, 13 mg, 14 mg, 15 mg, 20 mg, 25 mg, 30 mg, 40 mg, 50 mg, 60 mg, 70 mg, 80 mg, 90 mg, 100 mg, 125 mg, 150 mg, 175 mg, or at least 200 mg. In another embodiment, the daily dose is 0.05-1 mg, 1-2 mg, 2-4 mg, 1-5 mg, 5-7.5 mg, 7.5-10 mg, 10-15 mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, or 45-50 mg, 50-75 mg, 75-100 mg, 100-125 mg, 125-150 mg, 150-175 mg, 175-200 mg, or more than 200 mg.

In another embodiment, a single dose of Compound 1 administered to a patient is selected from about: 0.05 mg, 0.1 mg, 0.5, mg, 1 mg, 2 mg, 3 mg, 4 mg, 5 mg, 6 mg, 7 mg, 8 mg, 9 mg, 10 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, or 50 mg.

In another embodiment, a single dose of Compound 1 administered to a patient is selected from about: 0.05-1 mg, 1-2 mg, 2-4 mg, 1-5 mg, 5-7.5 mg, 7.5-10 mg, 10-15 mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, 45-50 mg, 50-75 mg, 75-100 mg, 100-125 mg, 125-150 mg, 150-175 mg, 175-200 mg, or more than 200 mg.

In one embodiment, the single dose is administered by a route selected from any one of: oral, buccal, or sublingual administration. In another embodiment, said single dose is administered by injection, e.g., subcutaneous, intramuscular, or intravenous. In another embodiment, said single dose is administered by inhalation or intranasal administration.

As a non-limited example, the dose of Compound 1 administered by intramuscular or subcutaneous injection may be about 0.05 to 50 mg per day to be administered in divided doses. A single dose of Compound 1 administered by intramuscular or subcutaneous injection may be about 0.05-6 mg, preferably about 1-4 mg, 1-3 mg, or 2 mg. Intra muscular or subcutaneous infusion may be preferable in those patients requiring division of injections into more than 10 doses daily. The continuous intramuscular or subcutaneous infusion dose may be 1 mg/hour daily and is generally increased according to response up to 4 mg/hour.

The fine particle dose of Compound 1 administered by pulmonary administration, e.g., inhalation using a pressurized metered dose inhaler (pMDI), dry powder inhaler (DPI), soft-mist inhaler, nebulizer, or other device, may be in the range of about, 0.5-15 mg, preferably about 0.5-8 mg or 2-6 mg. The Nominal Dose (ND), i.e., the amount of drug metered in the receptacle (also known as the Metered Dose), of Compound 1 administered by pulmonary administration may be, for example, in the range of 0.5-15 mg, 3-10 mg, 10-15 mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, or 45-50 mg.

Long-acting pharmaceutical compositions may be administered, 1, 2, 3, 4, 5, 6, 7, 8, 9, 10 or more than 10 times daily (preferably: 1 times per day), every other day, every 3 to 4 days, every week, or once every two weeks depending on half-life and clearance rate of the particular formulation.

Pharmaceutical compositions comprising (6aS)-6-methyl-5,6,6a,7-tetrahydro-4H-dibenzo[de,g]quinoline-10,11-diol and pharmaceutically-acceptable salts thereof can be administered by means that produces contact of the active agent with the agent's site of action. They can be administered by conventional means available for use in conjunction with pharmaceuticals in a dosage range of 0.001 to 1000 mg/kg of mammal body weight per day in a single dose or in divided doses. One dosage range is 0.01 to 500 mg/kg body weight per day in a single dose or in divided doses. Administration can be delivered as individual therapeutic agents or in a combination of therapeutic agents. They can be administered alone, but typically are administered with a pharmaceutically acceptable excipient selected on the basis of the chosen route of administration and standard pharmaceutical practice.

Indications Suitable for Treatment

The pharmaceutical compositions of the present invention may be employed to treat or reduce the symptoms associated with protein misfolding or conditions that would benefit from a reduction of such activity. Because (6aS)-6-methyl-5,6,6a,7-tetrahydro-4H-dibenzo[de,g]quinoline-10,11-diol modifies the protein misfolding leading to neurodegenerative diseases, preferred embodiments of the present invention include pharmaceutical compositions that have (6aS)-6-methyl-5,6,6a,7-tetrahydro-4H-dibenzo[de,g]quinoline-10,11-diol or a pharmaceutically acceptable salt thereof and a pharmaceutically acceptable excipient that provide a pharmacokinetic profile conducive to the treatment of neurodegenerative diseases.

Conditions suitable for treatment according to this invention include neurodegenerative diseases include Alzheimer's disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy).

Pharmacokinetic (PK) Profile

Various embodiments further include a pharmaceutical composition having (6aS)-6-methyl-5,6,6a,7-tetrahydro-4H-dibenzo[de,g]quinoline-10,11-diol or a pharmaceutically acceptable salt thereof and at least one pharmaceutically acceptable excipient, and a pharmacokinetic profile after administration to a mammal. When used at the appropriate dosages the described pharmacokinetic profiles provide levels of (6aS)-6-methyl-5,6,6a,7-tetrahydro-4H-dibenzo[de,g]quinoline-10,11-diol for the treatment of neurological diseases which include, Alzheimer's disease, Parkinson's Disease, multiple sclerosis, Huntington's Disease, ALS, spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sachs, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy); and preferably ALS, Huntington's disease, Parkinson's disease, and Friedreich's ataxia. In particular, the pharmaceutical compositions of the present invention provide a pharmacokinetic profile that provides (6aS)-6-methyl-5,6,6a,7-tetrahydro-4H-dibenzo[de,g]quinoline-10,11-diol to the site of biological action. Preferably in the central nervous system (CNS) of the subject. More preferably in the cerebral spinal fluid (CSF) and the brain of the subject.

One embodiment of the present invention includes a pharmaceutical composition having (6aS)-6-methyl-5,6,6a,7-tetrahydro-4H-dibenzo[de,g]quinoline-10,11-diol and at least one pharmaceutically acceptable excipient, and provides one or more pharmacokinetic properties including a t_{1/2} at about 0.1 to about 72 hours, a Tₘₐₓ at about 0 to about 24 hours, a Cₘₐₓ at about 1 ng/mL to about 50,000 ng/mL, a brain to plasma ratio in a mammal for (6aS)-6-methyl-5,6,6a,7-tetrahydro-4H-dibenzo[de,g]quinoline-10,11-diol of about 0.1 to about 6, a CSF to plasma ratio in a mammal for (6aS)-6-methyl-5,6,6a,7-tetrahydro-4H-dibenzo[de,g]quinoline-10,11-diol of about 0.1 to about 6, a bioavailability of about 0.1% to about 99% relative to intravenous administration, and/or an AUCₗₐₛₜ/Cₘₐₓ or AUCₗₐₛₜ/C₀ ratio from about 0.0001 to about 100 hours.

One embodiment of the present invention includes a pharmaceutical composition having (6aS)-6-methyl-5,6,6a,7-tetrahydro-4H-dibenzo[de,g]quinoline-10,11-diol and at least one pharmaceutically acceptable excipient, and provides a pharmacokinetic profile that includes a t_{1/2} at about 0.1 to about 72 hours after administration to mammal. In another embodiment, the pharmaceutical composition exhibits a pharmacokinetic profile that includes a t_{1/2} at about 0.3 to about 48 hours after intramuscular or subcutaneous administration. In another embodiment, the pharmaceutical composition exhibits a pharmacokinetic profile that includes a t_{1/2} at about 0.3 to about 25 hours after intramuscular or subcutaneous administration. In another embodiment, the pharmaceutical composition exhibits a pharmacokinetic profile that includes a t_{1/2} at about 0.3 to about 10 hours after intramuscular or subcutaneous administration. Another embodiment of the pharmaceutical composition exhibits a pharmacokinetic profile that includes a t_{1/2} at about 0.5 to about 7 hours after administration. In certain embodiments the pharmaceutical composition of exhibits a pharmacokinetic profile that includes a t_{1/2} at about 0.5 to about 7 hours after intramuscular or subcutaneous administration. Yet in other embodiments, the pharmaceutical composition of exhibits a pharmacokinetic profile that includes a t_{1/2} at about 0.5 to about 7 hours after sublingual or buccal administration. In another embodiment, the pharmaceutical composition of exhibits a pharmacokinetic profile that includes a t_{1/2} at about 0.5 to about 7 hours after intranasal administration. Another embodiment includes the pharmaceutical composition provides a t_{1/2} at about 0.05 to about 5 hours after intravenous administration. In another embodiment, the pharmaceutical composition exhibits a t_{1/2} at about 0.01 to about 30 hours after oral administration. In one aspect, the pharmaceutical composition exhibits a t_{1/2} at about 1 to about 30 hours after oral administration. In another aspect, the pharmaceutical composition exhibits a t_{1/2} at about 5 to about 25 hours after oral administration.

One embodiment of the present invention includes a pharmaceutical composition having (6aS)-6-methyl-5,6,6a,7-tetrahydro-4H-dibenzo[de,g]quinoline-10,11-diol and at least one pharmaceutically acceptable excipient, and provides a pharmacokinetic profile that includes a Tₘₐₓ at about 0 to about 24 hours after administration to a mammal. In another embodiment, the pharmaceutical composition exhibits a pharmacokinetic profile that includes a Tₘₐₓ at about 0.05 to about 10 hours after administration. In another embodiment, the pharmaceutical composition exhibits a pharmacokinetic profile that includes a Tₘₐₓ at about 0.05 to about 4 hours after administration. In another embodiment, the pharmaceutical composition of exhibits a pharmacokinetic profile that includes a Tₘₐₓ at about 0.05 to about 4 hours after intramuscular or subcutaneous administration. Another embodiment of the pharmaceutical composition exhibits a pharmacokinetic profile that includes a Tₘₐₓ at about 0.05 to about 4 hours after sublingual administration. In yet another embodiment, the pharmaceutical composition exhibits a pharmacokinetic profile that includes a Tₘₐₓ at about 0.05 to about 4 hours after intranasal administration. In another embodiment, the pharmaceutical composition of exhibits a Tₘₐₓ at about 0.1 to about 5 hours after oral administration.

One embodiment of the present invention includes a pharmaceutical composition having (6aS)-6-methyl-5,6,6a,7-tetrahydro-4H-dibenzo[de,g]quinoline-10,11-diol and at least one pharmaceutically acceptable excipient, and provides a pharmacokinetic profile that includes a Cₘₐₓ at about 1 ng/mL to about 50,000 ng/mL after administration to a mammal. In another embodiment, the pharmaceutical composition provides a pharmacokinetic profile that includes a Cₘₐₓ at about 1 ng/mL to about 5000 ng/mL after administration. In another embodiment, the pharmaceutical composition provides a pharmacokinetic profile that includes a Cₘₐₓ at about 1 ng/mL to about 5000 ng/mL after intramuscular or subcutaneous administration. In yet another embodiment, the pharmaceutical composition provides a pharmacokinetic profile that includes a Cₘₐₓ at about 1 ng/mL to about 5000 ng/mL after sublingual administration. In another embodiment, the pharmaceutical composition provides a pharmacokinetic profile that includes a Cₘₐₓ at about 1 ng/mL to about 5000 ng/mL after intranasal administration. In another embodiment, the pharmaceutical composition provides a pharmacokinetic profile that includes a Cₘₐₓ at about 1000 ng/mL. In another embodiment, the pharmaceutical composition provides a Cₘₐₓ at about 5 ng/mL to about 50 ng/mL after oral administration.

An embodiment of the present invention further includes a pharmaceutical composition that includes (6aS)-6-methyl-5,6,6a,7-tetrahydro-4H-dibenzo[de,g]quinoline-10,11-diol or a pharmaceutically acceptable salt thereof and at least one pharmaceutically acceptable excipient, and provides a pharmacokinetic profile that includes a Tₘₐₓ at about 0 to about 10 hours, a Cₘₐₓ at about 1 ng/mL to about 50,000 ng/mL, and a t_{1/2} at about 0.1 to about 15 hours after administration to a mammal.

The present invention also includes embodiments of a pharmaceutical composition that includes (6aS)-6-methyl-5,6,6a,7-tetrahydro-4H-dibenzo[de,g]quinoline-10,11-diol and at least one pharmaceutically acceptable excipient, and provides a brain to plasma ratio in a mammal for (6aS)-6-methyl-5,6,6a,7-tetrahydro-4H-dibenzo[de,g]quinoline-10,11-diol of about 0.1 to about 6 following administration to a mammal. Another embodiment of the present invention includes a pharmaceutical composition that includes (6aS)-6-methyl-5,6,6a,7-tetrahydro-4H-dibenzo[de,g]quinoline-10,11-diol and at least one pharmaceutically acceptable excipient, and provides a CSF to plasma ratio in a mammal for (6aS)-6-methyl-5,6,6a,7-tetrahydro-4H-dibenzo[de,g]quinoline-10,11-diol of about 0.1 to about 6 following administration to a mammal.

A further embodiment of the present invention includes a pharmaceutical composition that includes (6aS)-6-methyl-5,6,6a,7-tetrahydro-4H-dibenzo[de,g]quinoline-10,11-diol and at least one pharmaceutically acceptable excipient, and provides a bioavailability of about 0.1% to about 99% relative to intravenous administration. In another embodiment, the pharmaceutical composition provides an oral bioavailability of about 1% to about 40% relative to intravenous administration. In another embodiment, the pharmaceutical composition provides an oral bioavailability of about 5% to about 30% relative to intravenous administration. In yet another embodiment, the pharmaceutical composition provides a intramuscular or subcutaneous bioavailability of about 40% to about 99% relative to intravenous administration. In another embodiment, the pharmaceutical composition of claim 25 provides a sublingual or buccal bioavailability of about 20% to about 80% relative to intravenous administration. In another embodiment, the pharmaceutical composition provides an intranasal bioavailability of about 20% to about 80% relative to intravenous administration.

Embodiments of the present invention also encompass a pharmaceutical composition that includes (6aS)-6-methyl-5,6,6a,7-tetrahydro-4H-dibenzo[de,g]quinoline-10,11-diol and at least one pharmaceutically acceptable excipient, and provides a bioavailability of about 0.1% to about 99% relative to intramuscular or subcutaneous administration. In one embodiment, the pharmaceutical composition provides an oral bioavailability of about 1% to about 40% relative to intramuscular or subcutaneous administration. In another embodiment, the pharmaceutical composition of provides an oral bioavailability of about 5% to about 30% relative to intramuscular or subcutaneous administration. In another embodiment, the pharmaceutical composition provides a sublingual bioavailability of about 20% to about 80% relative to intramuscular or subcutaneous administration. In yet another embodiment, the pharmaceutical composition provides an intranasal bioavailability of about 20% to about 80% relative to intramuscular or subcutaneous administration.

Another embodiment includes a pharmaceutical composition having (6aS)-6-methyl-5,6,6a,7-tetrahydro-4H-dibenzo[de,g]quinoline-10,11-diol or a pharmaceutically acceptable salt thereof and at least one pharmaceutically acceptable excipient, and provides an AUCₗₐₛₜ/Cₘₐₓ ratio from about 0.0001 to about 100 hours. In one aspect, the pharmaceutical composition provides a provides an AUCₗₐₛₜ/Cₘₐₓ ratio from about 0.0001 to about 50 hours after administration. In another aspect, the pharmaceutical composition provides a provides an AUCₗₐₛₜ/Cₘₐₓ ratio from about 0.0001 to about 20 hours after administration. In one embodiment, the pharmaceutical composition provides a provides an AUCₗₐₛₜ/Cₘₐₓ ratio from about 0.0001 to about 100 hours after intramuscular or subcutaneous administration. In another aspect, the pharmaceutical composition provides a provides an AUCₗₐₛₜ/Cₘₐₓ ratio from about 0.01 to about 20 hours after intramuscular or subcutaneous administration. In yet another aspect, the pharmaceutical composition provides an AUCₗₐₛₜ/Cₘₐₓ ratio from about 0.0001 to about 100 hours after sublingual or buccal administration. In other embodiments, the pharmaceutical composition provides an AUCₗₐₛₜ/Cₘₐₓ ratio from about 0.01 to about 20 hours after sublingual or buccal administration. In another embodiment, pharmaceutical composition provides an AUCₗₐₛₜ/Cₘₐₓ ratio from about 0.0001 to about 100 hours after intranasal administration. In another embodiment, the pharmaceutical composition provides an AUCₗₐₛₜ/Cₘₐₓ ratio from about 0.01 to about 20 hours after intranasal administration. In other embodiments, the pharmaceutical composition of provides an AUCₗₐₛₜ/Cₘₐₓ ratio from about 0.0001 to about 100 hours after oral administration. In another embodiment, the pharmaceutical composition provides an AUCₗₐₛₜ/Cₘₐₓ ratio from about 0.01 to about 20 hours after oral administration. In another embodiment, the pharmaceutical composition provides an AUCₗₐₛₜ/Cₘₐₓ ratio from about 0.0001 to about 100 hours after intravenous administration. In another embodiment, the pharmaceutical composition provides an AUCₗₐₛₜ/Cₘₐₓ ratio from about 0.0001 to about 20 hours after intravenous administration.

### EXAMPLES

### Example 1: Preparation of (6aS)-6-methyl-5,6,6a,7-tetrahydro-4H-dibenzo[de,g]quinoline-10,11-diol Hydrochloride

The following examples contain detailed methods of preparing (6aS)-6-methyl-5,6,6a,7-tetrahydro-4H-dibenzo[de,g]quinoline-10,11-diol. The general synthetic scheme is presented for illustrative purposes only and is not intended as a restriction on the scope of the invention. All parts are by weight and temperatures are in Degrees Celsius unless otherwise indicated.

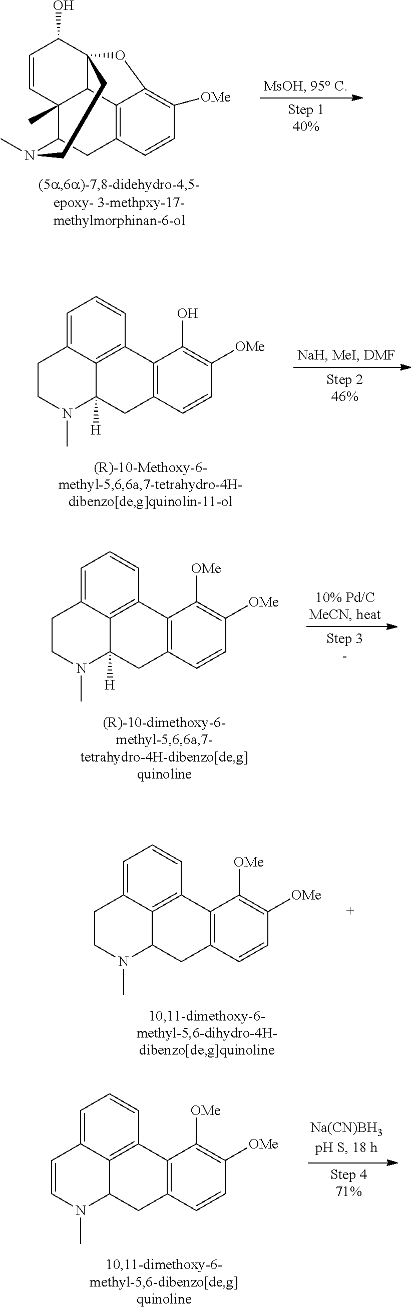

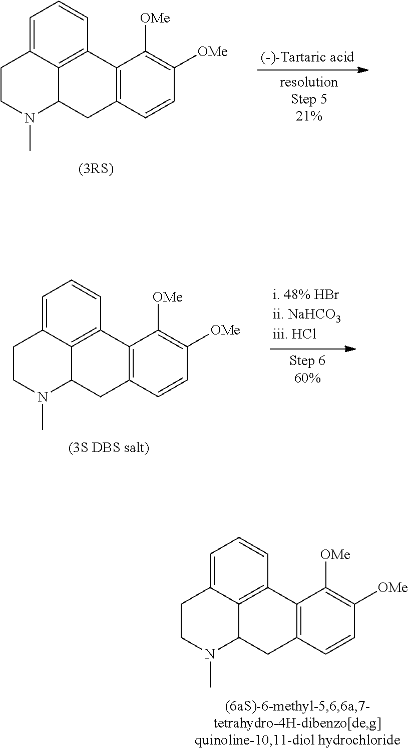 [SMILES: COc1ccc2c(c1OC)-c1cccc3c1C(C2)N(C)CC3 ; COc1ccc2c(c1OC)-c1cccc3c1C(C2)N(C)CC3 ; COc1ccc2c(c1OC)-c1cccc3c1C(C2)N(C)CC3]

### Preparation of (R)-10-Methoxy-6-methyl-5,6,6a,7-tetrahydro-4H-dibenzo[de,g]quinolin-11-ol

A mixture of (5a,6a)-7,8-didehydro-4,5-epoxy-3-methoxy-17-methylmorphinan-6-ol (10.0 g, 33.4 mmol) and methanesulfonic acid (40 ml) was heated to 100° C. After 30 minutes, the temperature was lowered to 65° C. and after a further 30 minutes, the reaction was left to cool to room temperature. The mixture was diluted with water (150 ml) and then basified with ammonium hydroxide (with cooling in an ice bath to keep the temperature <50° C.). The resultant suspension was cooled to room temperature, collected by filtration and washed with water. The solid was dissolved in dichloromethane (50 ml) and separated from residual water.

The resultant solution was passed through a pad of silica eluting with 5% methanol/dichloromethane. Concentration of appropriate fractions gave the desired product as a white foam (3.8 g, 40%).

### Preparation of (R)-10,11-dimethoxy-6-methyl-5,6,6a,7-tetrahydro-4H-dibenzo[de,g]quinoline

To a solution of (R)-10-Methoxy-6-methyl-5,6,6a,7-tetrahydro-4H-dibenzo[de,g]quinolin-11-ol (5.0 g, 17.8 mmol) in dimethylformamide (25 ml) was added 60% sodium hydride (0.75 g, 18.8 mmol). After stirring for 20 minutes, methyl iodide (2.66 g, 18.8 mmol) was added dropwise over 10 minutes. The reaction mixture was stirred for 20 minutes and then poured onto water (70 ml) and extracted with methyl t-butyl ether (150 ml). The organic layer was separated, washed with 2M sodium hydroxide solution (3×50 ml) then brine (50 ml) and concentrated to a pink oil which was purified on a plug of silica eluted 5% methanol/dichloromethane. Concentration of appropriate fractions gave the desired product (2.43 g, 46%).

### Preparation of 10,11-dimethoxy-6-methyl-5,6-dihydro-4H-dibenzo[de,g]quinoline and 10,11-dimethoxy-6-methyl-6H-dibenzo[de,g]quinoline

A solution of (R)-10,11-dimethoxy-6-methyl-5,6,6a,7-tetrahydro-4H-dibenzo[de,g]quinoline (18.47 g, 65.9 mmol) in acetonitrile (550 ml) was treated with a slurry of 10% Pd/C in acetonitrile (450 ml) and the mixture heated at reflux overnight and then allowed to cool. The catalyst was collected by filtration, washed with acetonitrile and the combined filtrate concentrated to a dark oil (10.6 g) which was used without further purification.

### Preparation of (3RS)

A mixture of 10,11-dimethoxy-6-methyl-5,6-dihydro-4H-dibenzo[de,g]quinoline and 10,11-dimethoxy-6-methyl-6H-dibenzo[de,g]quinoline (10.6 g, 36.1 mmol) was dissolved in absolute ethanol (500 ml) and treated with sodium cyanoborohoydride (11 g, 175 mmol). Part of a solution of acetyl chloride (8.8 g, 0.113 mol) in absolute ethanol (100 ml) was added until the mixture became cloudy and the mixture stirred at room temperature. After stirring overnight, the reaction was found to be around 80% complete by LC-MS; stirring was continued and further aliquots of the acetyl chloride solution added at intervals. Once the reaction was found to contain <2% starting material by LCMS, the mixture was concentrated and the residue diluted with water (300 ml) and basified with potassium carbonate. After extraction with ethyl acetate (2×300 ml), the organic layer was washed with brine, concentrated and then purified by chromatography (silica gel, 3→10% methanol:dichloromethane) (7.53 g, 71%).

### Preparation of (S)-10,11-dimethoxy-6-methyl-5,6,6a,7-tetrahydro-4H-dibenzo[de,g]quinoline (2R,3R)-2,3-bis(benzoyloxy)succinate (3S DBS salt)

To a solution of (−)-Dibenzoyl-L-tartaric acid (8.73 g, 24.4 mmol) in ethyl acetate (80 ml) was added a solution of (3RS) (7.20 g, 24.4 mmol) in ethyl acetate (30 ml) over 15 minutes. The resultant precipitate was heated to reflux and isopropanol (56 ml) added to give a dark solution. After heating for 15 minutes, the solution was allowed to slowly cool to room temperature for two hours. It was then further cooled in an ice bath for an hour and the resultant precipitate collected by filtration (2.6 g). A second crop was also collected (1.4 g). The two were combined and heated to reflux in ethyl acetate (35 ml); isopropanol (20 ml) was then added slowly until a solution was obtained. This was allowed to slowly cool and the product collected by filtration in 3 crops, all of which had >95% ee by chiral NMR (3.28 g, 21%).

### Preparation of (6aS)-6-methyl-5,6,6a, 7-tetrahydro-4H-dibenzo[de,g]quinoline-10,11-diol hydrochloride

A slurry of (3S DBS salt) (4.4 g, 6.7 mmol) was treated with potassium carbonate until pH 8. The mixture was extracted with ethyl acetate (2×40 ml) and the combined extracts dried and concentrated to an orange/brown oil (1.90 g). This was dissolved in acetic acid (30 ml), treated with 48% hydrobromic acid (30 ml) and the mixture heated under reflux for 5 hours under nitrogen. After cooling to room temperature overnight, the mixture was concentrated to ca 10 ml and then carefully basified by the slow addition of saturated sodium bicarbonate solution. The product was extracted with ethyl acetate (2×60 ml), washed with brine, dried over sodium sulphate and concentrated. The resultant blue foam was dissolved in absolute ethanol (40 ml) and treated with concentrated hydrochloric acid (1.1 ml) and activated carbon (600 mg). After heating under reflux for 40 minutes, the mixture was hot filtered and the filter bed washed with boiling absolute ethanol (80 ml). The combined filtrate was concentrated and the white solid thus obtained triturated with cold acetone (15 ml) to give the desired product. (1.08 g, 60%).

In Vivo Bioactivity

The following general methods are used in order to describe and demonstrate biological activity and potential therapy usage of compounds of the present invention only, and are not to be construed in any way as limiting the scope of the invention. Final yield 210 g (92%).

### Example 2: Pharmacokinetic and Brain Penetration Study Following Single Dose of (6aS)-6-methyl-5,6,6a,7-tetrahydro-4H-dibenzo[de,g]quinoline-10,11-diol Hydrochloride Via Subcutaneous Administration in Male C57BL/6J Mouse

(6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol pharmacokinetic profiles were evaluated in male C57BL/6J mice (N=3). 9 male C57BL/6J mice following administration of a single subcutaneous dose of 4.40 mg/kg. The vehicle for the subcutaneous formulation contained “0.1% sodium metabisulfite solution”. During this PK study, there were no obvious drug-related changes in clinical signs and behavioral responses. There was no animal death related to drug administration.

Animals

Male C57BL/6J mice were purchased from Si Bei Fu Laboratory Animal Technology Co. Ltd (Beijing, China). The animals were about 8 weeks old with body weights of 20-30 g on the dosing date. The animals were housed in a 12-hour light/12-hour dark cycle environment and had free access to food and water. All animals were fasted overnight and fed after 4 hours collection.

Study Design

(6aS)-6-methyl-5,6,6a,7-tetrahydro-4H-dibenzo[de,g]quinoline-10,11-diol hydrochloride hemihydrate was administered with subcutaneous dose (4.40 mg/kg, free base) at 5 mL/kg. For group 1, plasma samples were collected from dorsal metatarsal vein at each time point after subcutaneous administrations. For group 2, plasma, CSF and brain samples were collected at 0.167, 0.5 h post dose.

| | | Dose Level | Dose Level | | | | | |
|---|---|---|---|---|---|---|---|---|
| | | (mg/kg) (salt | (mg/kg) (salt | Dose Level | Dose | | | |
| | | hemihydrate | anhydrous | (mg/kg) | Volume | Conc. | | No. of |
| Group | Treatment | form) | form) | (free base) | (mL/kg) | (mg/mL) | Route | Animals |
| 1 | (6aS)-6-methyl- | 5.15 | 5 | 4.40 | 5 | 0.880 | Subcutaneous | 3/male |
| | 5,6,6a,7-tetrahydro-4H- | | | | | | | |
| | dibenzo[de,g]quinoline- | | | | | | | |
| | 10,11-diol | | | | | | | |
| | hydrochloride | | | | | | | |
| 2 | (6aS)-6-methyl- | 5.15 | 5 | 4.40 | 5 | 0.880 | Subcutaneous | 6/male |
| | 5,6,6a,7-tetrahydro-4H- | | | | | | | |
| | dibenzo[de,g]quinoline- | | | | | | | |
| | 10,11-diol | | | | | | | |
| | hydrochloride | | | | | | | |

Dosage and Administration

Preparation of dosing for subcutaneous administration (4.40 mg/kg, Group 1): 1.22 mg of (6aS)-6-methyl-5,6,6a,7-tetrahydro-4H-dibenzo[de,g]quinoline-10,11-diol hydrochloride hemihydrate was dissolved in 1.185 mL of 0.1% sodium metabisulfite solution with vortexing to obtain a solution with concentration at 0.880 mg/mL of (6aS)-6-methyl-5,6,6a,7-tetrahydro-4H-dibenzo[de,g]quinoline-10,11-diol.

Preparation of dosing for subcutaneous administration (4.40 mg/kg, Group 2): 1.25 mg of (6aS)-6-methyl-5,6,6a,7-tetrahydro-4H-dibenzo[de,g]quinoline-10,11-diol hydrochloride hemihydrate was dissolved in 1.214 mL of 0.1% sodium metabisulfite solution with vortexing to obtain a solution with concentration at 0.880 mg/mL of (6aS)-6-methyl-5,6,6a,7-tetrahydro-4H-dibenzo[de,g]quinoline-10,11-diol.

Dose Sample Validation (Subcutaneous):

| | |
|---|---|
| Step 1 | Take 20 μL subcutaneous dose to 180 μL DMSO |
| | to make first 10× dilution (S1) |
| Step 2 | Take 10 μL of solution from S1 to 990 μL 50% |
| | ACN in water to make second 100× dilution (S2) |
| Step 3 | Take 3 μL of solution from S2 to 30 μL blank |
| | Plasma to make third 10× dilution (S3) |

The total dose solution was diluted 10000×. The concentrations of dose formulation were measured by LC-MS, and the results shown in the table below:

| | | Dilution | Nominal | Measured | Mean | Accuracy | SD | CV |
|---|---|---|---|---|---|---|---|---|
| Route | Sample Name | Factor | (mg/mL) | (mg/mL) | (mg/mL) | (%) | (mg/mL) | (%) |
| Subcutaneous | Dose_G1_1 | 10000 | 0.880 | 0.921 | 0.880 | 100 | 0.0518 | 5.89 |
| G1 | Dos_G1_2 | 10000 | | 0.821 | | | | |
| | Dose_G1_3 | 10000 | | 0.897 | | | | |
| Subcutaneous | Dose_G2_1 | 10000 | 0.880 | 0.979 | 0.953 | 108 | 0.0259 | 2.72 |
| G2 | Dose_G2_2 | 10000 | | 0.928 | | | | |
| | Dose_G2_3 | 10000 | | 0.951 | | | | |

Sample Collection

For Group 1 (4.40 mg/kg), blood samples were collected from each animal at 0.0833, 0.167, 0.25, 0.5, 1, 2 hr post dose. For Group 2 (4.40 mg/kg), blood, CSF and brain samples were collected from 3 mice at two time points: 0.167 hr and 0.5 hr post dose.

For plasma samples, about 30 μL or 300 μL of blood samples were collected from each animal via dorsal metatarsal vein or heart. These blood samples were placed into the tubes containing K2EDTA. And centrifugation at 3000 g for 10 minutes in a 4° C. centrifuge. The completion time from collection was less than 30 min. The stabilizing agent of ascorbic acid at 200 mg/mL was added in a proportion of 10% in plasma. The plasma samples were stored in a freezer at −75±15° C. prior to analysis. The completion time from collection was less than 60 min.

For brain and CSF samples, The animals were fully exsanguinated with a rising concentration of CO2 gas prior to CSF and brain collection.

For CSF collection, incision was made by scissors on the neck of the rats to expose the foramen magnum. A syringe fitted with a venoclysis needle was used to sample the CSF, the tip of the needle was inserted into the membrane of foramen magnum about 1-2 mm, and a negative pressure was applied by pulling a stopcock a small length of the syringe to extract CSF fluids into a tube. The stopcock was further actuated to until a sufficient amount of CSF was collected. There should be no blood in the CSF fluid. A stabilizing agent of ascorbic acid at 200 mg/mL was added in a proportion of 10% in CSF.

For brain collection. Brain samples were collected at an adopted time point, and washed 3 times with cold saline, then dried with clean gauze and put into the weighed tube, quick frozen in an ice box and kept at −75±15° C. All brain samples were weighted and homogenized with phosphate buffered saline (PBS) by brain weight (g) to PBS volume (mL) ratio 1:3 before analysis. The stabilizing agent of ascorbic acid at 200 mg/mL was added in a proportion of 10% in grain homogenate. The actual concentration was the detected value multiplied by the dilution factor. Brain samples were collected at designed time point, quickly homogenized and kept at −75±15° C.

### Preparation of Standards

About 1 mg (6aS)-6-methyl-5,6,6a,7-tetrahydro-4H-dibenzo[de,g]quinoline-10,11-diol hydrochloride hemihydrate and (6aR)-6-methyl-5,6,6a,7-tetrahydro-4H-dibenzo[de,g]quinoline-10,11-diol hydrochloride hemihydrate was dissolved in DMSO to obtain a 1 mg/mL standard stock solution (free base).

Calibration standard working solutions were prepared at concentrations of 2, 5, 10, 20, 50, 100, 500, 1000, 5000 and 10000 ng/mL by serial dilution of the standard stock solution in 50% acetonitrile in water. Quality control working solutions at concentrations of 5, 10, 500 and 8000 ng/mL were prepared by serial dilution of the standard stock solution in 50% acetonitrile in water. These QC samples were prepared on the day of analysis in the same way as calibration standards.

Sample Treatment

For plasma and brain samples, 3 μL of each calibration standard working solution (2, 5, 10, 20, 50, 100, 500, 1000, 5000 and 10000 ng/mL) was added to 30 μL of the blank C57BL/6J mouse plasma or brain homogenate (with 10% 200 mg/mL ascorbic acid) to achieve calibration standards of 0.2-1000 ng/mL (0.2, 0.5, 1, 2, 5, 10, 50, 100, 500, 1000 ng/mL) in a total volume of 33 μL. Quality Control (QC) samples at 0.5 ng/mL (low-1), 1 ng/mL (low-2), 50 ng/mL (mid), 800 ng/mL (high) for plasma were prepared independently for those used for the calibration curves. These QC samples were prepared on the day of analysis in the same way as calibration standards. The total volumes of 33 μL of standards, 33 μL of QC samples, and 33 μL of unknown samples (30 μL of unknown plasma or brain homogenate (with 10% 200 mg/mL ascorbic acid) with 3 μL 50% acetonitrile) were added to 200 μL of acetonitrile containing IS to precipitate proteins. The samples were subsequently vortexed for 30 sec. After centrifugation at 4° C., 4700 rpm for 30 min, and supernatant was diluted 2 times with water, 10μL of the diluted supernatant was injected into the LC-MS/MS system for quantitative analysis.

For CSF samples, 2 μL of each calibration standard working solution (2, 5, 10, 20, 50, 100, 500, 1000, 5000 and 10000 ng/mL) was added to 20 μL of the artificial CSF (with 10% 200 mg/mL ascorbic acid) to achieve calibration standards of 0.2-1000 ng/mL (0.2, 0.5, 1, 2, 5, 10, 50, 100, 500, 1000 ng/mL) in a total volume of 22 μL. Quality Control (QC) samples at 0.5 ng/mL (low-1), 1 ng/mL (low-2), 50 ng/mL (mid), 800 ng/mL (high) for plasma were prepared independently for those used for the calibration curves. These QC samples were prepared on the day of analysis in the same way as calibration standards. The total volumes of 22 μL of standards, 22 μL of QC samples and 22 μL of unknown samples (20 μL of unknown CSF (with 10% 200 mg/mL ascorbic acid) with 2 μL 50% acetonitrile) were added to 200 μL of acetonitrile containing IS to precipitate proteins. Then the samples were vortexed for 30 sec. After centrifugation at 4° C., 4700 rpm for 30 min, and supernatant was diluted 2 times with water, 10 μL of the diluted supernatant was injected into the LC-MS/MS system for quantitative analysis.

All the samples were processed on ice.

LC-MS/MS Conditions

The LC-MS/MS system consisted of two Shimadzu LC-30AD pumps, a DGU-20A5R degasser, a Rack changer II and LCMS-8060 LC/MS/MS instrument mass spectrometer.

Chromatographic separation was performed on a DAICEL CORPORATION CHIRALCEL OD-3R Particle size 3 μm 4.6 mm×150 mm column at room temperature. The mobile phase was composed of A: Water (5 mM Ammonium Acetate); B: Ethanol (5 mM Ammonium Acetate). The flow rate was 0.7 mL/min. The injection volume was 10 μL.

Positive mode electrospray ionization (ESI) was performed to obtain a protonated ion of (6aR)-6-methyl-5,6,6a,7-tetrahydro-4H-dibenzo[de,g]quinoline-10,11-diol, (6aS)-6-methyl-5,6,6a,7-tetrahydro-4H-dibenzo[de,g]quinoline-10,11-diol, and Dexamethasone (IS). A multiple reaction monitoring (MRM) method was selected for quantitative analysis. The optimized transitions were 268.20191.10, 268.20191.10 and 393.20-373.20 for (6aR)-6-methyl-5,6,6a,7-tetrahydro-4H-dibenzo[de,g]quinoline-10,11-diol, (6aS)-6-methyl-5,6,6a,7-tetrahydro-4H-dibenzo[de,g]quinoline-10,11-diol, and Dexamethasone, respectively. The instrument parameters were set as follows: ion spray voltage: 8060; heating gas flow: 10 L/min; interface temperature: 250° C.; DL temperature: 220° C.; heat block temperature: 400° C.; driving gas flow: 10 L/min. (6aR)-6-methyl-5,6,6a,7-tetrahydro-4H-dibenzo[de,g]quinoline-10,11-diol, (6aS)-6-methyl-5,6,6a,7-tetrahydro-4H-dibenzo[de,g]quinoline-10,11-diol, and Dexamethasone dependent parameters are listed as follows:

| | (6aR)-6-methyl- | (6aS)-6-methyl- | |
|---|---|---|---|
| | 5,6,6a,7- | 5,6,6a,7- | |
| | tetrahydro- | tetrahydro- | |
| | 4H-dibenzo | 4H-dibenzo | |
| | [de,g]quinoline- | [de,g]quinoline- | Dexamethasone |
| Compound ID | 10,11-diol | 10,11-diol | (IS) |
| Transition | 268.20→191.10 | 268.20→191.10 | 393.20→373.20 |
| Dwell | 100 | 100 | 50 |
| Time (msec) | | | |
| Q1 Pre Bias (v) | −10 | −10 | −25 |
| CE | −30 | −30 | −10 |
| Q3 Pre Bias (v) | −19 | −19 | −18 |

Data acquisition was performed by Lab Solutions version 5.89 Software (Shimadzu). All concentration data was reported with 3 significant figures. Data statistics were performed using Excel 2010 software. The pharmacokinetic parameters of (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol were calculated using a non-compartmental approach with Phoenix™ WinNonlin®.

Results

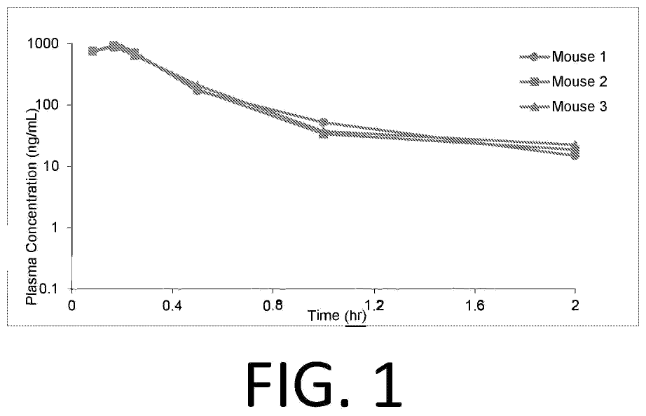

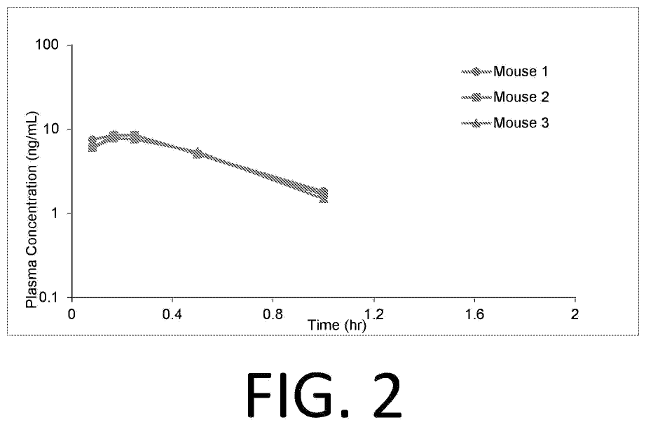

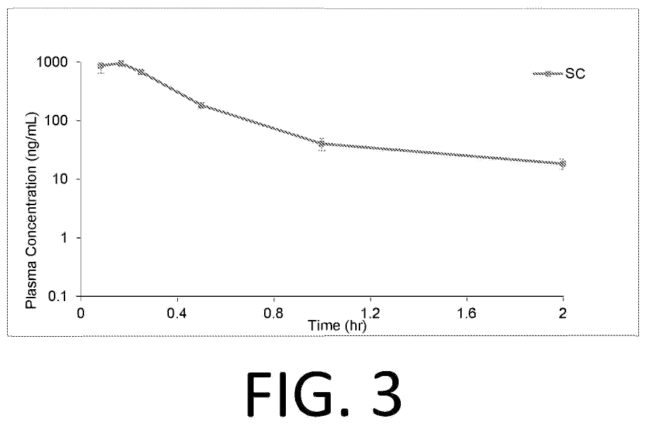

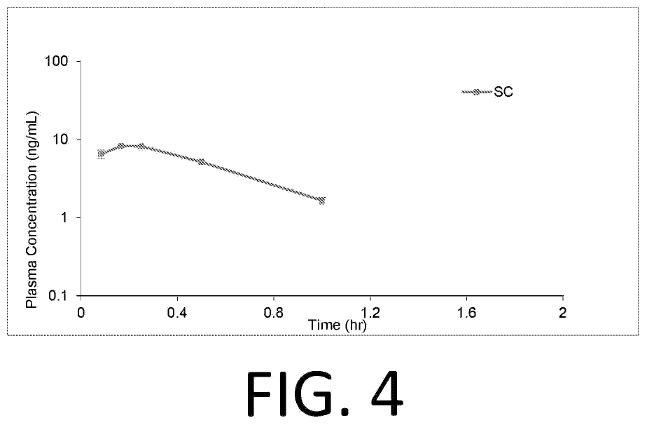

The (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol plasma concentrations vs. time profiles are shown in FIGS. 1-4.

The pharmacokinetic parameters for (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol in mice following subcutaneous administration were calculated by non-compartmental analysis (Phoenix™ WinNonlin® 8.0), and a linear trapezoidal algorithm was used for AUC calculation. The resulting pharmacokinetic parameters for (6aS)-6-methyl-5,6,6a,7-tetrahydro-4H-dibenzo[de,g]quinoline-10,11-diol are as follows:

| | t_{1/2} | Tₘₐₓ | Cₘₐₓ | AUCₗₐₛₜ | AUC_{∞} | MRTInf | AUCₗₐₛₜ/D | AUCₗₐₛₜ/Cₘₐₓ |
|---|---|---|---|---|---|---|---|---|
| Animal | (h) | (h) | (ng/mL) | (h*ng/mL) | (h*ng/mL) | (h) | (h*mg/mL) | (h) |
| Mouse 1 | 0.442 | 0.0833 | 1147 | 417 | 426 | 0.384 | 80.9 | 0.364 |
| Mouse 2 | 0.502 | 0.167 | 931 | 361 | 375 | 0.434 | 70.1 | 0.388 |
| Mouse 3 | 0.513 | 0.167 | 888 | 363 | 379 | 0.474 | 70.4 | 0.409 |
| Mean | 0.486 | 0.139 | 989 | 380 | 393 | 0.431 | 73.8 | 0.387 |
| SD | 0.0381 | 0.0483 | 139 | 31.6 | 28.5 | 0.0451 | 6.14 | 0.023 |

The resulting pharmacokinetic parameters for (6aR)-6-methyl-5,6,6a,7-tetrahydro-4H-dibenzo[de,g]quinoline-10,11-diol are as follows:

| | t_{1/2} | Tₘₐₓ | Cₘₐₓ | AUCₗₐₛₜ | AUC_{∞} | MRTInf | AUCₗₐₛₜ/D | AUCₗₐₛₜ/Cₘₐₓ |
|---|---|---|---|---|---|---|---|---|
| Animal | (h) | (h) | (ng/mL) | (h*ng/mL) | (h*ng/mL) | (h) | (h*mg/mL) | (h) |
| Mouse 1 | 0.338 | 0.167 | 8.56 | 5.10 | 5.99 | 0.542 | 139 | 0.596 |
| Mouse 2 | 0.319 | 0.25 | 8.43 | 4.91 | 5.67 | 0.526 | 134 | 0.582 |
| Mouse 3 | 0.312 | 0.167 | 7.78 | 4.80 | 5.47 | 0.515 | 131 | 0.617 |
| Mean | 0.323 | 0.195 | 8.26 | 4.94 | 5.71 | 0.528 | 135 | 0.598 |
| SD | 0.0133 | 0.0479 | 0.418 | 0.156 | 0.264 | 0.0135 | 4.25 | 0.017 |

The resulting ratios of (6aS)-6-methyl-5,6,6a,7-tetrahydro-4H-dibenzo[de,g]quinoline-10,11-diol between plasma and brain following subcutaneous administration to Male C57BL/6J mice at 4.40 mg/kg are as follows:

| | | | | Ratio | Mean Ratio |
|---|---|---|---|---|---|
| Time | | Brain | Plasma | (Brain/ | (Brain/ |
| (h) | Animal | (ng/g) | (ng/mL) | Plasma) | Plasma) |
| 0.167 h | Mouse 4 | 1802 | 776 | 2.32 | 2.72 |
| | Mouse 5 | 2991 | 1003 | 2.98 | |
| | Mouse 6 | 2446 | 857 | 2.85 | |
| 0.5 h | Mouse 4 | 1059 | 257 | 4.12 | 5.32 |
| | Mouse 5 | 1117 | 205 | 5.45 | |
| | Mouse 6 | 975 | 153 | 6.38 | |

The resulting ratios of (6aS)-6-methyl-5,6,6a,7-tetrahydro-4H-dibenzo[de,g]quinoline-10,11-diol between plasma and CSF following subcutaneous administration to male C57BL/6J mice at 4.40 mg/kg are as follows:

| | | | | Ratio | Mean Ratio |
|---|---|---|---|---|---|
| Time | | CSF | Plasma | (CSF/ | (CSF/ |
| (h) | Animal | (ng/mL) | (ng/mL) | Plasma) | Plasma) |
| 0.167 h | Mouse 4 | 20.5 | 776 | 0.0264 | 0.0245 |
| | Mouse 5 | 21.3 | 1003 | 0.0213 | |
| | Mouse 6 | 22.2 | 857 | 0.0259 | |
| 0.5 h | Mouse 4 | 9.59 | 257 | 0.0373 | 0.0389 |
| | Mouse 5 | 7.73 | 205 | 0.0377 | |
| | Mouse 6 | 6.38 | 153 | 0.0417 | |

The resulting ratios of (6aR)-6-methyl-5,6,6a,7-tetrahydro-4H-dibenzo[de,g]quinoline-10,11-diol between plasma and brain following subcutaneous administration to male C57BL/6J mice at 4.40 mg/kg are as follows:

| | | | | Ratio | Mean Ratio |
|---|---|---|---|---|---|
| Time | | Brain | Plasma | (Brain/ | (Brain/ |
| (h) | Animal | (ng/g) | (ng/mL) | Plasma) | Plasma) |
| 0.167 h | Mouse 4 | 36.6 | 8.67 | 4.22 | 5.54 |
| | Mouse 5 | 57.9 | 10.1 | 5.74 | |
| | Mouse 6 | 50.6 | 7.62 | 6.65 | |
| 0.5 h | Mouse 4 | 38.0 | 5.43 | 6.99 | 9.27 |
| | Mouse 5 | 42.1 | 4.60 | 9.15 | |
| | Mouse 6 | 38.7 | 3.32 | 11.7 | |

The resulting ratios of (6aS)-6-methyl-5,6,6a,7-tetrahydro-4H-dibenzo[de,g]quinoline-10,11-diol between plasma and CSF following subcutaneous administration to male C57BL/6J mice at 4.40 mg/kg are as follows:

| | | | | Ratio | Mean Ratio |
|---|---|---|---|---|---|
| Time | | CSF | Plasma | (CSF/ | (CSF/ |
| (h) | Animal | (ng/mL) | (ng/mL) | Plasma) | Plasma) |
| 0.167 h | Mouse 4 | BLOQ | 8.67 | NA | NA |
| | Mouse 5 | BLOQ | 10.1 | NA | |
| | Mouse 6 | BLOQ | 7.62 | NA | |
| 0.5 h | Mouse 4 | BLOQ | 5.43 | NA | NA |
| | Mouse 5 | BLOQ | 4.60 | NA | |
| | Mouse 6 | BLOQ | 3.32 | NA | |

NA=Not available; BLOQ=Below quantifiable limit of 1 ng/mL for CSF.

(6aS)-6-methyl-5,6,6a,7-tetrahydro-4H-dibenzo[de,g]quinoline-10,11-diol plasma concentrations following subcutaneous administration to male C57BL/6J mice at 4.40 mg/kg:

| Time | Conc. (ng/mL) | | | Mean | SD | CV |
|---|---|---|---|---|---|---|
| (h) | Mouse 1 | Mouse 2 | Mouse 3 | (ng/mL) | (ng/mL) | (%) |
| 0.0833 | 1147 | 760 | 750 | 886 | 226 | 25.6 |
| 0.167 | 1092 | 931 | 888 | 970 | 107 | 11.1 |
| 0.25 | 721 | 706 | 648 | 692 | 38.8 | 5.61 |
| 0.5 | 169 | 181 | 211 | 187 | 21.5 | 11.5 |
| 1 | 52.0 | 33.7 | 37.2 | 41.0 | 9.70 | 23.7 |
| 2 | 14.9 | 18.7 | 22.5 | 18.7 | 3.79 | 20.3 |

(6aR)-6-methyl-5,6,6a,7-tetrahydro-4H-dibenzo[de,g]quinoline-10,11-diol plasma concentrations following subcutaneous administration to male C57BL/6J Mice at 0.0367 mg/kg:

| Time | Conc. (ng/mL) | | | Mean | SD | CV |
|---|---|---|---|---|---|---|
| (h) | Mouse 1 | Mouse 2 | Mouse 3 | (ng/mL) | (ng/mL) | (%) |
| 0.0833 | 7.46 | 6.01 | 6.03 | 6.50 | 0.829 | 12.8 |
| 0.167 | 8.56 | 8.38 | 7.78 | 8.24 | 0.407 | 4.94 |
| 0.25 | 8.50 | 8.43 | 7.56 | 8.16 | 0.523 | 6.41 |
| 0.5 | 5.06 | 5.03 | 5.37 | 5.15 | 0.187 | 3.62 |
| 1 | 1.82 | 1.66 | 1.49 | 1.65 | 0.165 | 10.0 |
| 2 | BLOQ | BLOQ | BLOQ | NA | NA | NA |

(6aS)-6-methyl-5,6,6a,7-tetrahydro-4H-dibenzo[de,g]quinoline-10,11-diol plasma concentrations following subcutaneous administration to male C57BL/6J Mice at 4.40 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.167 | 776 | 1003 | 857 | 879 | 115 | 13.1 |
| 0.5 | 257 | 205 | 153 | 205 | 52.1 | 25.4 |

(6aS)-6-methyl-5,6,6a, 7-tetrahydro-4H-dibenzo[de,g]quinoline-10,11-diol CSF concentrations following subcutaneous administration to male C57BL/6J Mice at 4.40 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.167 | 20.5 | 21.3 | 22.2 | 21.3 | 0.823 | 3.86 |
| 0.5 | 9.59 | 7.73 | 6.38 | 7.90 | 1.61 | 20.4 |

(6aS)-6-methyl-5,6,6a, 7-tetrahydro-4H-dibenzo[de,g]quinoline-10,11-diol brain concentrations following subcutaneous administration to male C57BL/6J Mice at 4.40 mg/kg:

| Time | Conc. (ng/mL) | | | Conc. (ng/g) | | | Mean | SD | CV |
|---|---|---|---|---|---|---|---|---|---|
| (h) | Sort 1 | Sort 2 | Sort 3 | Sort 1 | Sort 2 | Sort 3 | (ng/g) | (ng/g) | (%) |
| 0.167 | 450 | 748 | 612 | 1802 | 2991 | 2446 | 2413 | 596 | 24.7 |
| 0.5 | 265 | 279 | 244 | 1059 | 1117 | 975 | 1050 | 71.4 | 6.80 |

(6aR)-6-methyl-5,6,6a,7-tetrahydro-4H-dibenzo[de,g]quinoline-10,11-diol plasma concentrations following subcutaneous administration to male C57BL/6J Mice at 0.0367 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.167 | 8.67 | 10.1 | 7.62 | 8.79 | 1.24 | 14.1 |
| 0.5 | 5.43 | 4.60 | 3.32 | 4.45 | 1.07 | 24.0 |

(6aR)-6-methyl-5,6,6a,7-tetrahydro-4H-dibenzo[de,g]quinoline-10,11-diol CSF concentrations following subcutaneous administration to male C57BL/6J mice at 0.0367 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.167 | BLOQ | BLOQ | BLOQ | NA | NA | NA |
| 0.5 | BLOQ | BLOQ | BLOQ | NA | NA | NA |

(6aR)-6-methyl-5,6,6a,7-tetrahydro-4H-dibenzo[de,g]quinoline-10,11-diol brain concentrations following subcutaneous administration to male C57BL/6J mice at 0.0367 mg/kg:

| Time | Conc. (ng/mL) | | | Conc. (ng/g) | | | Mean | SD | CV |
|---|---|---|---|---|---|---|---|---|---|
| (h) | Sort 1 | Sort 2 | Sort 3 | Sort 1 | Sort 2 | Sort 3 | (ng/g) | (ng/g) | (%) |
| 0.167 | 7.32 | 11.6 | 10.1 | 36.6 | 57.9 | 50.6 | 48.4 | 10.8 | 22.4 |
| 0.5 | 7.60 | 8.41 | 7.75 | 38.0 | 42.1 | 38.7 | 39.6 | 2.17 | 5.48 |

### Example 3: Pharmacokinetic Study Following Single Dose of (6aS)-6-methyl-5,6,6a,7-tetrahydro-4H-dibenzo[de,g]quinoline-10,11-diol Hydrochloride Via IV, Subcutaneous, PO and IN administration at 0.880 mg/kg, 0.880 mg/kg, 4.40 mg/kg and 1.76 mg/kg in Male SD Rats

(6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol pharmacokinetic profiles were evaluated in male SD Rats (N=3). 12 male SD Rats were administered with single IV dose of 0.880 mg/kg, subcutaneous dose of 0.880 mg/kg, PO dose of 4.40 mg/kg, and IN dose of 1.76 mg/kg. The vehicle for the IV, subcutaneous, PO and IN formulation contained “0.1% sodium metabisulfite solution”. During this PK study, there were no obvious drug-related changes in clinical signs and behavioral responses. There was no animal death related to drug administration.

Animals

Male SD rats were purchased from Si Bei Fu Laboratory Animal Technology Co. Ltd (Beijing, China). The animals were about 8 weeks old with body weights of 200-300 g on the dosing date. The animals were housed in a 12-hour light/12-hour dark cycle environment and had free access to food and water. All animals were fasted overnight and fed after 4 hours collection.

Study Design

(6aS)-6-methyl-5,6,6a,7-tetrahydro-4H-dibenzo[de,g]quinoline-10,11-diol hydrochloride hemihydrate was administered with IV and subcutaneous dose (0.880 mg/kg, free base) at 5 mL/kg. (6aS)-6-methyl-5,6,6a,7-tetrahydro-4H-dibenzo[de,g]quinoline-10,11-diol hydrochloride hemihydrate was administered with PO dose (4.40 mg/kg, free base) at 5 mL/kg. (6aS)-6-methyl-5,6,6a,7-tetrahydro-4H-dibenzo[de,g]quinoline-10,11-diol hydrochloride hemihydrate was administered with IN dose (1.76 mg/kg, free base) at 0.2 mL/kg. Plasma samples were collected from jugular vein at each time point after IV, subcutaneous, PO and IN administrations.

| | | | Dose Level | Dose | | | | | |
|---|---|---|---|---|---|---|---|---|---|
| | | Dose Level | (mg/kg) | Level | Dose Level | | | | |
| | | (mg/kg) (salt | (salt | (mg/kg) | (mg/kg) | Dose | | | |
| | | hemihydrate | anhydrous | (free | (anhydrous | Volume | Conc. | | No. of |
| Group | Treatment | Form) | form) | base) | salt) | (mL/kg) | (mg/mL) | Route | Animals |
| 1 | (6aS)-6-methyl- | 1.03 | 1 | 0.880 | 1.0 | 5 | 0.176 | IV | 3/male |
| | 5,6,6a,7-tetrahydro-4H- | | | | | | | | |
| | dibenzo[de,g]quin- | | | | | | | | |
| | oline-10,11-diol | | | | | | | | |
| | hydrochloride | | | | | | | | |
| 2 | (6aS)-6-methyl- | 1.03 | 1 | 0.880 | 1.0 | 5 | 0.176 | Subcutaneous | 3/male |
| | 5,6,6a,7-tetrahydro-4H- | | | | | | | | |
| | dibenzo[de,g]quin- | | | | | | | | |
| | oline-10,11-diol | | | | | | | | |
| | hydrochloride | | | | | | | | |
| 3 | (6aS)-6-methyl- | 5.15 | 5 | 4.40 | 5.0 | 5 | 0.880 | PO | 3/male |
| | 5,6,6a,7-tetrahydro-4H- | | | | | | | | |
| | dibenzo[de,g]quin- | | | | | | | | |
| | oline-10,11-diol | | | | | | | | |
| | hydrochloride | | | | | | | | |
| 4 | (6aS)-6-methyl- | 2.06 | 2 | 1.76 | 2.0 | 0.2 | 8.80 | IN (split | 3/male |
| | 5,6,6a,7-tetrahydro-4H- | | | | | | | to dose to | |
| | dibenzo[de,g]quino- | | | | | | | 2 nostrils) | |
| | line-10,11-diol | | | | | | | | |
| | hydrochloride | | | | | | | | |

Dosage and Administration

Preparation of dosing for IV and subcutaneous administration (0.880 mg/kg): 2.17 mg of (6aS)-6-methyl-5,6,6a,7-tetrahydro-4H-dibenzo[de,g]quinoline-10,11-diol hydrochloride hemihydrate was dissolved in 10.537 mL of 0.1% sodium metabisulfite solution with vortexing to obtain a solution with concentration at 0.176 mg/mL of (6aS)-6-methyl-5,6,6a,7-tetrahydro-4H-dibenzo[de,g]quinoline-10,11-diol.

Preparation of dosing for PO administration (4.40 mg/kg): 5.53 mg of (6aS)-6-methyl-5,6,6a,7-tetrahydro-4H-dibenzo[de,g]quinoline-10,11-diol hydrochloride hemihydrate was dissolved in 5.371 mL of 0.1% sodium metabisulfite solution with vortexing to obtain a solution with concentration at 0.880 mg/mL of (6aS)-6-methyl-5,6,6a,7-tetrahydro-4H-dibenzo[de,g]quinoline-10,11-diol.

Preparation of dosing for IN administration (1.76 mg/kg): 2.37 mg of (6aS)-6-methyl-5,6,6a,7-tetrahydro-4H-dibenzo[de,g]quinoline-10,11-diol hydrochloride hemihydrate was dissolved in 10.537 mL of 0.1% sodium metabisulfite solution with vortexing to obtain a solution with concentration at 8.80 mg/mL of (6aS)-6-methyl-5,6,6a,7-tetrahydro-4H-dibenzo[de,g]quinoline-10,11-diol.

Dose Sample Validation (IV and Subcutaneous):

| | |
|---|---|
| Step 1 | Take 20 μL IV and subcutaneous dose to 180 μL |
| | DMSO to make first 10× dilution (S1). |
| Step 2 | Take 100 μL of solution from S1 to 900 μL 50% |
| | ACN in water to make second 10× dilution (S2). |
| Step 3 | Take 2 μL of solution from S2 to 20 μL blank |
| | Plasma to make third 10× dilution (S3). |

The total dose solution was diluted 1000×.

Dose Sample Validation (IN):

| | |
|---|---|
| Step 1 | Take 20 μL IN dose to 180 μL DMSO |
| | to make first 10× dilution (S1). |
| Step 2 | Take 10 μL of solution from S1 to 990 μL 50% |
| | ACN in water to make second 100× dilution (S2). |
| Step 3 | Take 100 μL of solution from S2 to 900 μL 50% |
| | ACN in water to make third 10× dilution (S3). |
| Step 4 | Take 2 μL of solution from S3 to 20 μL blank |
| | Plasma to make fourth 10× dilution (S4). |

Totally dose solution was diluted 100000×.

The concentrations of dose formulation were measured by LC-MS, and the results shown in the table below:

| | | Dilution | Nominal | Measured | Mean | Accuracy | SD | CV |
|---|---|---|---|---|---|---|---|---|
| Route | Sample Name | Factor | (mg/mL) | (mg/mL) | (mg/mL) | (%) | (mg/mL) | (%) |
| IV&SC | Dose_IV&SC_1 | 1000 | 0.176 | 0.125 | 0.129 | 73.1 | 0.00770 | 5.98 |
| 0.880 | Dose_IV&SC_2 | 1000 | | 0.123 | | | | |
| mg/kg | Dose_IV&SC_3 | 1000 | | 0.138 | | | | |
| PO | Dose_PO_1 | 10000 | 0.880 | 0.625 | 0.619 | 70.4 | 0.0164 | 2.65 |
| 4.40 | Dose_PO_2 | 10000 | | 0.632 | | | | |
| mg/kg | Dose_PO_3 | 10000 | | 0.601 | | | | |
| IN | Dose_IN_1 | 100000 | 8.80 | 6.00 | 6.09 | 69.3 | 0.340 | 5.58 |
| 1.76 | Dose_IN_2 | 100000 | | 6.47 | | | | |
| mg/kg | Dose_IN_3 | 100000 | | 5.82 | | | | |

Sample Collection

For IV and subcutaneous (0.880 mg/kg) administration, blood samples were collected from each animal at 0.0333, 0.0833, 0.167, 0.25, 0.5, 1, 2, 4, 8 h post dose.

For PO (4.40 mg/kg) administration, blood samples were collected from each animal at 0.0833, 0.167, 0.25, 0.5, 0.75, 1, 2, 4, 8 h post dose.

For IN (1.76 mg/kg) administration, blood samples were collected from each animal at 0.0333, 0.0833, 0.167, 0.25, 0.5, 1, 2, 4, 8 h post dose.

About 200 μL of blood samples were collected from each animal via jugular vein. These blood samples were placed into the tubes containing K2EDTA. And centrifugation at 3000 g for 10 minutes in a 4° C. centrifuge. The completion time from collection was less than 30 min. The stabilizing agent of ascorbic acid at 200 mg/mL was added in a proportion of 10% in plasma. The plasma samples were stored in a freezer at −75±15° C. prior to analysis. The completion time from collection was less than 60 min.

### Preparation of Standards

About 1 mg (6aS)-6-methyl-5,6,6a,7-tetrahydro-4H-dibenzo[de,g]quinoline-10,11-diol hydrochloride hemihydrate and (6aR)-6-methyl-5,6,6a,7-tetrahydro-4H-dibenzo[de,g]quinoline-10,11-diol hydrochloride hemihydrate was dissolved in DMSO to obtain a 1 mg/mL standard stock solution (free base).

Calibration standard working solutions were prepared at concentrations of 2, 5, 10, 20, 50, 100, 500, 1000, 5000 and 10000 ng/mL by serial dilution of the standard stock solution in 50% acetonitrile in water. Quality control working solutions at concentrations of 5, 10, 20, 500 and 8000 ng/mL were prepared by serial dilution of the standard stock solution in 50% acetonitrile in water. These QC samples were prepared on the day of analysis in the same way as calibration standards.

Sample Treatment

For plasma samples, 2 μL of each calibration standard working solution (2, 5, 10, 20, 50, 100, 500, 1000, 5000 and 10000 ng/mL) was added to 20 μL of the blank SD rat plasma (with 10% 200 mg/mL ascorbic acid) to achieve calibration standards of 0.2-1000 ng/mL (0.2, 0.5, 1, 2, 5, 10, 50, 100, 500, 1000 ng/mL) in a total volume of 22 μL. Quality Control (QC) samples at 0.5 ng/mL (low-1), 1 ng/mL (low-2) and 2 ng/mL (low-3), 50 ng/mL (mid), 800 ng/mL (high) for plasma were prepared independently for those used for the calibration curves. These QC samples were prepared on the day of analysis in the same way as calibration standards. The total volumes of 22 μL of standards, 22 μL of QC samples and 22 μL of unknown samples (20 μL of unknown plasma (with 10% 200 mg/mL ascorbic acid) with 2 μL 50% acetonitrile) were added to 200 μL of acetonitrile containing IS to precipitate proteins. Then the samples were vortexed for 30 sec. After centrifugation at 4° C., 4700 rpm for 30 min, and supernatant was diluted 2 times with water, 10 μL of the diluted supernatant was injected into the LC-MS/MS system for quantitative analysis.

All the samples were processed on ice.

LC-MS/MS Conditions

The LC-MS/MS system consisted of two Shimadzu LC-30AD pumps, a DGU-20A5R degasser, a Rack changer II and LCMS-8060 LC/MS/MS instrument mass spectrometer.

Chromatographic separation was performed on a DAICEL CORPORATION CHIRALCEL OD-3R Particle size 3 μm 4.6 mm×150 mm column at room temperature. The mobile phase was composed of A: Water (5 mM Ammonium Acetate); B: Ethanol (5 mM Ammonium Acetate). The flow rate was 0.5 mL/min. The injection volume was 10 μL.

Positive mode electrospray ionization (ESI) was performed to obtain a protonated ion of (6aR)-6-methyl-5,6,6a,7-tetrahydro-4H-dibenzo[de,g]quinoline-10,11-diol, (6aS)-6-methyl-5,6,6a,7-tetrahydro-4H-dibenzo[de,g]quinoline-10,11-diol, and Dexamethasone (IS). A multiple reaction monitoring (MRM) method was selected for quantitative analysis. The optimized transitions were 268.20→237.15, 268.20→237.15 and 393.20→373.20 for (6aR)-6-methyl-5,6,6a,7-tetrahydro-4H-dibenzo[de,g]quinoline-10,11-diol, (6aS)-6-methyl-5,6,6a,7-tetrahydro-4H-dibenzo[de,g]quinoline-10,11-diol, and Dexamethasone, respectively. The instrument parameters were set as follows: ion spray voltage: 8060; heating gas flow: 10 L/min; interface temperature: 250° C.; DL temperature: 220° C.; heat block temperature: 400° C.; drying gas flow: 10 L/min. (6aR)-6-methyl-5,6,6a,7-tetrahydro-4H-dibenzo[de,g]quinoline-10,11-diol, (6aS)-6-methyl-5,6,6a,7-tetrahydro-4H-dibenzo[de,g]quinoline-10,11-diol, and Dexamethasone dependent parameters are listed as follows:

| | (6aR)-6-methyl- | (6aS)-6-methyl- | |
|---|---|---|---|
| | 5,6,6a,7- | 5,6,6a,7- | |
| | tetrahydro- | tetrahydro- | |
| | 4H-dibenzo | 4H-dibenzo | |
| | [de,g]quinoline- | [de,g]quinoline- | Dexamethasone |
| Compound ID | 10,11-diol | 10,1-diol | (IS) |
| Transition | 268.20→237.15 | 268.20→237.15 | 393.20→373.20 |
| Dwell | 10.2 | 10.2 | 50 |
| Time (msec) | | | |
| Q1 Pre Bias (v) | −13 | −13 | −25 |
| CE | −18 | −18 | −10 |
| Q3 Pre Bias (v) | −16 | −16 | −18 |

Data acquisition was performed by Lab Solutions version 5.89 Software (Shimadzu). All concentration data was reported with 3 significant figures. Data statistics were performed using Excel 2010 software. The pharmacokinetic parameters of (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol were calculated using a non-compartmental approach with Phoenix™ WinNonlin®.

Results

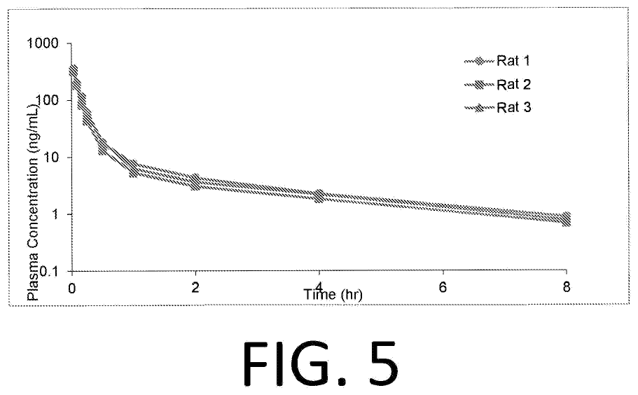

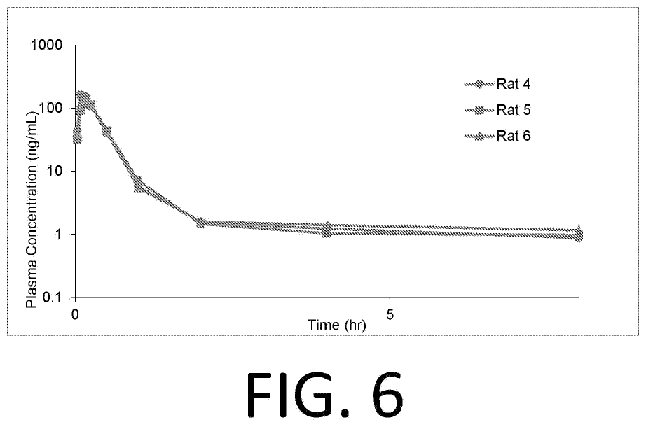

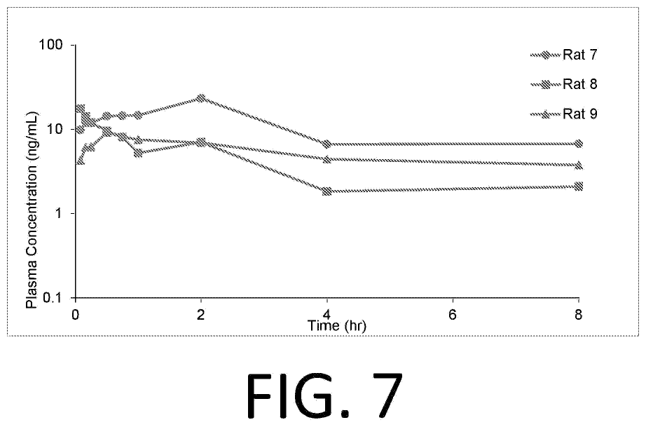

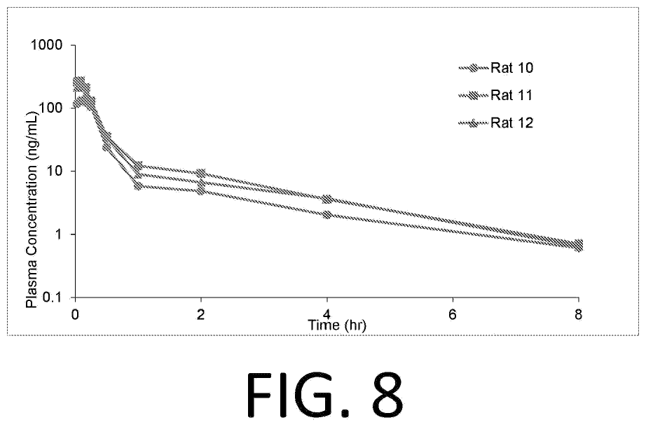

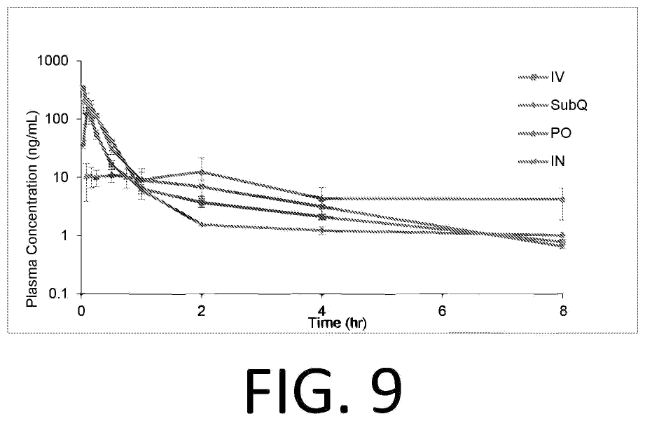

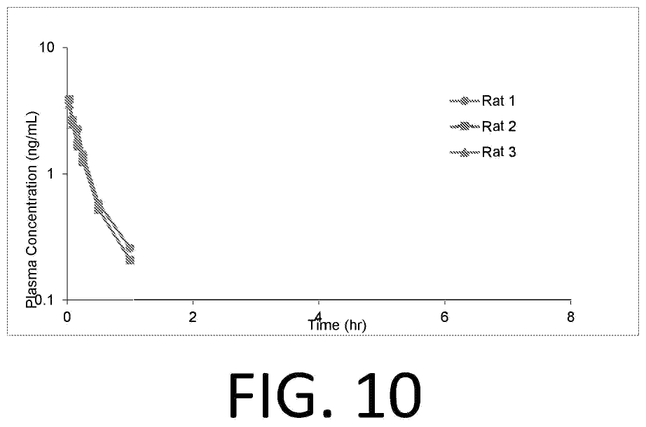

The (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol plasma concentrations vs. time profiles are shown in FIGS. 5-10.

The pharmacokinetic parameters for (6aS)-6-methyl-5,6,6a,7-tetrahydro-4H-dibenzo[de,g]quinoline-10,11-diol in Male SD Rats for IV at 0.880 mg/kg, subcutaneous at 0.880 mg/kg, PO at 4.40 mg/kg and IN at 1.76 mg/kg, were used to calculated by employing a non-compartmental analysis (Phoenix™ WinNonlin® 8.0). The linear trapezoidal algorithm was used for AUC calculation.

The resulting pharmacokinetic parameters for (6aS)-6-methyl-5,6,6a,7-tetrahydro-4H-dibenzo[de,g]quinoline-10,11-diol following IV administration to Male SD Rats at 0.880 mg/kg:

| | | | | | | | | | AUCₗₐₛₜ/ |
|---|---|---|---|---|---|---|---|---|---|
| | Cl | t_{1/2} | C₀ | AUCₗₐₛₜ | AUC_{Inf} | MRT_{Inf} | AUCₗₐₛₜ/D | Vₛₛ | C₀ |
| Animal | (mL/min/kg) | (h) | (ng/mL) | (h*ng/mL) | (h*ng/mL) | (h) | (h*mg/mL) | (L/kg) | (h) |
| Rat 1 | 169 | 2.66 | 527 | 83.2 | 86.6 | 1.26 | 94.6 | 12.8 | 0.158 |
| Rat 2 | 209 | 2.78 | 464 | 67.3 | 70.1 | 1.22 | 76.5 | 15.4 | 0.145 |
| Rat 3 | 175 | 2.66 | 476 | 80.6 | 83.6 | 1.18 | 91.6 | 12.4 | 0.169 |
| Mean | 185 | 2.70 | 489 | 77.1 | 80.1 | 1.22 | 87.6 | 13.5 | 0.157 |
| SD | 21.6 | 0.0690 | 33.5 | 8.54 | 8.81 | 0.0401 | 9.70 | 1.63 | 0.012 |

The resulting pharmacokinetic parameters for (6aS)-6-methyl-5,6,6a,7-tetrahydro-4H-dibenzo[de,g]quinoline-10,11-diol following subcutaneous administration to Male SD Rats at 0.880 mg/kg:

| | t_{1/2} | Tₘₐₓ | Cₘₐₓ | AUCₗₐₛₜ | AUC_{Inf} | MRTInf | AUClast/D | AUCₗₐₛₜ/ | F |
|---|---|---|---|---|---|---|---|---|---|
| Animal | (h) | (h) | (ng/mL) | (h*ng/mL) | (h*ng/mL) | (h) | (h*mg/mL) | Cₘₐₓ (h) | (%) |
| Rat 4 | 0.812 | 0.0833 | 162 | 70.2 | 71.2 | 0.873 | 79.8 | 0.433 | 89.0 |
| Rat 5 | 0.733 | 0.167 | 143 | 66.9 | 67.9 | 0.919 | 76.0 | 0.468 | 84.8 |
| Rat 6 | 0.839 | 0.167 | 119 | 67.0 | 68.4 | 1.06 | 76.1 | 0.563 | 85.4 |
| Mean | 0.795 | 0.139 | 141 | 68.0 | 69.2 | 0.951 | 77.3 | 0.488 | 86.4 |
| SD | 0.0552 | 0.0483 | 21.2 | 1.89 | 1.80 | 0.0985 | 2.15 | 0.067 | 2.25 |

The resulting ratios of (6aS)-6-methyl-5,6,6a,7-tetrahydro-4H-dibenzo[de,g]quinoline-10,11-diol following PO administration to Male SD Rats at 4.40 mg/kg:

| | t_{1/2} | Tₘₐₓ | Cₘₐₓ | AUCₗₐₛₜ | AUC_{Inf} | MRTInf | AUClast/D | AUCₗₐₛₜ/ | F |
|---|---|---|---|---|---|---|---|---|---|
| Animal | (h) | (h) | (ng/mL) | (h*ng/mL) | (h*ng/mL) | (h) | (h*mg/mL) | Cₘₐₓ (h) | (%) |
| Rat 7 | 3.93 | 2 | 23.2 | 88.4 | 127 | 6.26 | 20.1 | 3.810 | 23.0 |
| Rat 8 | 4.12 | 0.0833 | 17.5 | 32.5 | 45.0 | 5.71 | 7.39 | 1.857 | 8.44 |
| Rat 9 | 7.49 | 0.5 | 9.17 | 42.1 | 82.9 | 11.0 | 9.58 | 4.591 | 10.9 |
| Mean | 5.18 | 0.861 | 16.7 | 54.4 | 84.8 | 7.64 | 12.4 | 3.420 | 14.1 |
| SD | 2.00 | 1.01 | 7.08 | 29.9 | 40.8 | 2.88 | 6.79 | 1.408 | 7.76 |

The resulting ratios of (6aS)-6-methyl-5,6,6a,7-tetrahydro-4H-dibenzo[de,g]quinoline-10,11-diol following IN administration to Male SD Rats at 1.76 mg/kg:

| | t_{1/2} | Tₘₐₓ | Cₘₐₓ | AUCₗₐₛₜ | AUC_{Inf} | MRTInf | AUClast/D | AUCₗₐₛₜ/ | F |
|---|---|---|---|---|---|---|---|---|---|
| Animal | (h) | (h) | (ng/mL) | (h*ng/mL) | (h*ng/mL) | (h) | (h*mg/mL) | Cₘₐₓ (h) | (%) |
| Rat 10 | 1.18 | 0.0833 | 130 | 69.2 | 70.2 | 1.06 | 39.3 | 0.532 | 43.8 |
| Rat 11 | 1.21 | 0.0833 | 269 | 116 | 117 | 1.02 | 66.0 | 0.431 | 73.3 |
| Rat 12 | 1.34 | 0.0833 | 230 | 97.5 | 98.7 | 1.09 | 55.4 | 0.424 | 61.6 |
| Mean | 1.24 | 0.0833 | 209 | 94.3 | 95.4 | 1.06 | 53.6 | 0.462 | 59.6 |
| SD | 0.0814 | 0.00 | 71.4 | 23.7 | 23.8 | 0.0373 | 13.4 | 0.061 | 14.8 |

As shown in the tables below, the plasma concentrations of (6aR)-6-methyl-5,6,6a,7-tetrahydro-4H-dibenzo[de,g]quinoline-10,11-diol following IV administration to Male SD Rats at 0.880 mg/kg (Rats 1-3) and PO administration to Male SD Rats at 4.40 mg/kg (Rats 7-9), respectively, were not suitable for calculating PK parameters.

| Time | Conc. (ng/mL) | | | Mean | SD | CV |
|---|---|---|---|---|---|---|
| (h) | Rat 1 | Rat 2 | Rat 3 | (ng/mL) | (ng/mL) | (%) |
| 0.0333 | 3.85 | 3.87 | 3.54 | 3.75 | 0.183 | 4.88 |
| 0.0833 | 2.69 | 2.64 | 2.45 | 2.59 | 0.123 | 4.76 |
| 0.167 | 2.26 | 1.73 | 1.66 | 1.88 | 0.327 | 17.4 |
| 0.25 | 1.42 | 1.24 | 1.32 | 1.33 | 0.0903 | 6.79 |
| 0.5 | 0.582 | 0.520 | 0.575 | 0.559 | 0.0340 | 6.07 |
| 1 | 0.257 | 0.207 | BLOQ | 0.232 | NA | NA |
| 2 | BLOQ | BLOQ | BLOQ | NA | NA | NA |
| 4 | BLOQ | BLOQ | BLOQ | NA | NA | NA |
| 8 | BLOQ | BLOQ | BLOQ | NA | NA | NA |

| Time | Conc. (ng/mL) | | | Mean | SD | CV |
|---|---|---|---|---|---|---|
| (h) | Rat 7 | Rat 8 | Rat 9 | (ng/mL) | (ng/mL) | (%) |
| 0.0333 | BLOQ | BLOQ | BLOQ | NA | NA | NA |
| 0.0833 | BLOQ | BLOQ | BLOQ | NA | NA | NA |
| 0.167 | BLOQ | BLOQ | BLOQ | NA | NA | NA |
| 0.25 | BLOQ | BLOQ | BLOQ | NA | NA | NA |
| 0.5 | BLOQ | BLOQ | BLOQ | NA | NA | NA |
| 1 | BLOQ | BLOQ | BLOQ | NA | NA | NA |
| 2 | BLOQ | BLOQ | BLOQ | NA | NA | NA |
| 4 | BLOQ | BLOQ | BLOQ | NA | NA | NA |
| 8 | BLOQ | BLOQ | BLOQ | NA | NA | NA |

NA = Not available;
BLOQ = Below quantifiable limit of 0.2 or 0.5 ng/mL.

(6aS)-6-methyl-5,6,6a,7-tetrahydro-4H-dibenzo[de,g]quinoline-10,11-diol plasma concentrations following IV Administration to male SD Rats at 0.880 mg/kg:

| Time | Conc. (ng/mL) | | | Mean | SD | CV |
|---|---|---|---|---|---|---|
| (h) | Rat 1 | Rat 2 | Rat 3 | (ng/mL) | (ng/mL) | (%) |
| 0.0333 | 353 | 319 | 346 | 339 | 18.1 | 5.33 |
| 0.0833 | 193 | 181 | 214 | 196 | 16.7 | 8.52 |
| 0.167 | 115 | 80.3 | 104 | 100 | 17.7 | 17.8 |
| 0.25 | 59.1 | 43.2 | 61.0 | 54.4 | 9.77 | 17.9 |
| 0.5 | 17.8 | 13.1 | 18.3 | 16.4 | 2.90 | 17.7 |
| 1 | 7.60 | 5.28 | 6.23 | 6.37 | 1.16 | 18.3 |
| 2 | 4.32 | 3.06 | 3.65 | 3.67 | 0.628 | 17.1 |
| 4 | 2.26 | 1.83 | 2.20 | 2.10 | 0.235 | 11.2 |
| 8 | 0.880 | 0.683 | 0.767 | 0.777 | 0.0989 | 12.7 |

S(6aS)-6-methyl-5,6,6a,7-tetrahydro-4H-dibenzo[de,g]quinoline-10,11-diol plasma concentrations following subcutaneous administration to male SD Rats at 0.880 mg/kg:

| Time | Conc. (ng/mL) | | | Mean | SD | CV |
|---|---|---|---|---|---|---|
| (h) | Rat 4 | Rat 5 | Rat 6 | (ng/mL) | (ng/mL) | (%) |
| 0.0333 | 42.7 | 31.9 | 36.1 | 36.9 | 5.43 | 14.7 |
| 0.0833 | 162 | 91.0 | 104 | 119 | 37.5 | 31.5 |
| 0.167 | 151 | 143 | 119 | 138 | 16.5 | 12.0 |
| 0.25 | 107 | 112 | 113 | 110 | 3.11 | 2.81 |
| 0.5 | 40.5 | 43.8 | 44.9 | 43.1 | 2.25 | 5.23 |
| 1 | 5.76 | 7.07 | 5.44 | 6.09 | 0.860 | 14.1 |
| 2 | 1.52 | 1.46 | 1.60 | 1.53 | 0.0677 | 4.43 |
| 4 | 1.23 | 1.03 | 1.40 | 1.22 | 0.186 | 15.2 |
| 8 | 0.877 | 0.975 | 1.16 | 1.00 | 0.145 | 14.4 |

(6aS)-6-methyl-5,6,6a,7-tetrahydro-4H-dibenzo[de,g]quinoline-10,11-diol plasma concentrations following PO administration to male SD Rats at 4.40 mg/kg:

| Time | Conc. (ng/mL) | | | Mean | SD | CV |
|---|---|---|---|---|---|---|
| (h) | Rat 7 | Rat 8 | Rat 9 | (ng/mL) | (ng/mL) | (%) |
| 0.0833 | 9.85 | 17.5 | 4.26 | 10.6 | 6.67 | 63.2 |
| 0.167 | 12.0 | 14.1 | 6.12 | 10.7 | 4.13 | 38.5 |
| 0.25 | 11.9 | 12.0 | 6.17 | 10.0 | 3.32 | 33.2 |
| 0.5 | 14.3 | 9.58 | 9.17 | 11.0 | 2.84 | 25.8 |
| 0.75 | 14.5 | 8.07 | 8.14 | 10.2 | 3.70 | 36.2 |
| 1 | 14.6 | 5.23 | 7.53 | 9.13 | 4.90 | 53.7 |
| 2 | 23.2 | 7.03 | 6.93 | 12.4 | 9.39 | 75.7 |
| 4 | 6.65 | 1.83 | 4.42 | 4.30 | 2.41 | 56.1 |
| 8 | 6.73 | 2.09 | 3.77 | 4.20 | 2.35 | 55.9 |

(6aS)-6-methyl-5,6,6a,7-tetrahydro-4H-dibenzo[de,g]quinoline-10,11-diol plasma concentrations following IN administration to male SD Rats at 1.76 mg/kg:

| Time | Conc. (ng/mL) | | | Mean | SD | CV |
|---|---|---|---|---|---|---|
| (h) | Rat 10 | Rat 11 | Rat 12 | (ng/mL) | (ng/mL) | (%) |
| 0.0333 | 117 | 258 | 213 | 196 | 72.0 | 36.7 |
| 0.0833 | 130 | 269 | 230 | 209 | 71.4 | 34.1 |
| 0.167 | 122 | 210 | 165 | 166 | 44.1 | 26.7 |
| 0.25 | 106 | 129 | 108 | 114 | 13.0 | 11.4 |
| 0.5 | 23.9 | 35.7 | 33.3 | 31.0 | 6.25 | 20.2 |
| 1 | 5.79 | 12.2 | 8.93 | 8.96 | 3.19 | 35.6 |
| 2 | 4.82 | 9.16 | 6.64 | 6.87 | 2.18 | 31.7 |
| 4 | 2.02 | 3.57 | 3.68 | 3.09 | 0.929 | 30.0 |
| 8 | 0.607 | 0.706 | 0.640 | 0.651 | 0.0504 | 7.74 |

(6aR)-6-methyl-5,6,6a,7-tetrahydro-4H-dibenzo[de,g]quinoline-10,11-diol plasma concentrations following IV administration to male SD Rats at 0.880 mg/kg:

| Time | Conc. (ng/mL) | | | Mean | SD | CV |
|---|---|---|---|---|---|---|
| (h) | Rat 1 | Rat 2 | Rat 3 | (ng/mL) | (ng/mL) | (%) |
| 0.0333 | 3.85 | 3.87 | 3.54 | 3.75 | 0.183 | 4.88 |
| 0.0833 | 2.69 | 2.64 | 2.45 | 2.59 | 0.123 | 4.76 |
| 0.167 | 2.26 | 1.73 | 1.66 | 1.88 | 0.327 | 17.4 |
| 0.25 | 1.42 | 1.24 | 1.32 | 1.33 | 0.0903 | 6.79 |
| 0.5 | 0.582 | 0.520 | 0.575 | 0.559 | 0.0340 | 6.07 |
| 1 | 0.257 | 0.207 | BLOQ | 0.232 | NA | NA |
| 2 | BLOQ | BLOQ | BLOQ | NA | NA | NA |
| 4 | BLOQ | BLOQ | BLOQ | NA | NA | NA |
| 8 | BLOQ | BLOQ | BLOQ | NA | NA | NA |

(6aS)-6-methyl-5,6,6a,7-tetrahydro-4H-dibenzo[de,g]quinoline-10,11-diol plasma concentrations following PO administration to male SD Rats at 4.40 mg/kg:

| Time | Conc. (ng/mL) | | | Mean | SD | CV |
|---|---|---|---|---|---|---|
| (h) | Rat 7 | Rat 8 | Rat 9 | (ng/mL) | (ng/mL) | (%) |
| 0.0333 | BLOQ | BLOQ | BLOQ | NA | NA | NA |
| 0.0833 | BLOQ | BLOQ | BLOQ | NA | NA | NA |
| 0.167 | BLOQ | BLOQ | BLOQ | NA | NA | NA |
| 0.25 | BLOQ | BLOQ | BLOQ | NA | NA | NA |
| 0.5 | BLOQ | BLOQ | BLOQ | NA | NA | NA |
| 1 | BLOQ | BLOQ | BLOQ | NA | NA | NA |
| 2 | BLOQ | BLOQ | BLOQ | NA | NA | NA |
| 4 | BLOQ | BLOQ | BLOQ | NA | NA | NA |
| 8 | BLOQ | BLOQ | BLOQ | NA | NA | NA |

### Example 4: Pharmacokinetic and Brain Penetration Study Following Single Dose of (6aS)-6-methyl-5,6,6a,7-tetrahydro-4H-dibenzo[de,g]quinoline-10,11-diol Hydrochloride Via IN and PO Administration at 1.76 mg/kg and 10.56 mg/kg in Male SD Rats

(6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol pharmacokinetic profiles were evaluated in male SD rats (N=3). 12 male SD rats were administered with single IN and PO dose of 1.76 mg/kg and 10.56 mg/kg. The vehicle for the IN and PO formulation contained “0.1% sodium metabisulfite solution”.

Animals

Male SD rats were purchased from Si Bei Fu Laboratory Animal Technology Co. Ltd (Beijing, China). The animals were about 8 weeks old with body weights of 200-300 g on the dosing date. The animals were housed in a 12-hour light/12-hour dark cycle environment and had free access to food and water. All animals were fasted overnight and fed after 4 hours collection.

Study Design

(6aS)-6-methyl-5,6,6a,7-tetrahydro-4H-dibenzo[de,g]quinoline-10,11-diol hydrochloride hemihydrate was administered with an IN dose (1.76 mg/kg, free base) at 0.2 mL/kg, and a PO dose (10.56 mg/kg, free base) at 12 mL/kg. For group 1, plasma, CSF and brain samples were collected at 0.0833 and 0.5 h post dose. For group 2, plasma, CSF and brain samples were collected at 0.5 and 1 h post dose.

| | | Dose Level | Dose Level | | | | | |
|---|---|---|---|---|---|---|---|---|
| | | (mg/kg) (salt | (mg/kg) (salt | Dose Level | Dose | | | |
| | | hemihydrate | anhydrous | (mg/kg) | Volume | Conc. | | No. of |
| Group | Treatment | form) | form) | (free base) | (mL/kg) | (mg/mL) | Route | Animals |
| 1 | (6aS)-6-methyl- | 2.06 | 2 | 1.76 | 0.2 | 8.80 | IN (split | 3/male/time |
| | 5,6,6a,7- | | | | | | to dose to | point |
| | tetrahydro-4H- | | | | | | 2 nostril) | |
| | dibenzo[de,g]quin- | | | | | | | |
| | oline-10,11-diol | | | | | | | |
| | hydrochloride | | | | | | | |
| 2 | (6aS)-6-methyl- | 12.36 | 12 | 10.56 | 12 | 0.880 | PO | 3/male/time |
| | 5,6,6a,7- | | | | | | | point |
| | tetrahydro-4H- | | | | | | | |
| | dibenzo[de,g]quin- | | | | | | | |
| | oline-10,11-diol | | | | | | | |
| | hydrochloride | | | | | | | |

Dosage and Administration

Preparation of dosing for IN administration (1.76 mg/kg, Group 1): 3.86 mg of (6aS)-6-methyl-5,6,6a,7-tetrahydro-4H-dibenzo[de,g]quinoline-10,11-diol hydrochloride hemihydrate was dissolved in 0.375 mL of 0.1% sodium metabisulfite solution with vortexing to obtain a solution with concentration at 8.80 mg/mL of (6aS)-6-methyl-5,6,6a,7-tetrahydro-4H-dibenzo[de,g]quinoline-10,11-diol.

Preparation of dosing for PO administration (10.56 mg/kg, Group 2): 24.04 mg of (6aS)-6-methyl-5,6,6a,7-tetrahydro-4H-dibenzo[de,g]quinoline-10,11-diol hydrochloride hemihydrate was dissolved in 23.347 mL of 0.1% sodium metabisulfite solution with vortexing to obtain a solution with concentration at 0.880 mg/mL of (6aS)-6-methyl-5,6,6a,7-tetrahydro-4H-dibenzo[de,g]quinoline-10,11-diol.

Dose Sample Validation (IN and PO):

| | |
|---|---|
| Step 1 | Take 20 μL IN and PO dose to 180 μL DMSO to |
| | make first 10 X dilution (S1). |
| Step 2 | Take 10 μL of solution from S1 to 990 μL 50% |
| | ACN in water to make second 100 X dilution (S2). |
| Step 3 | Take 100 μL of solution from S2 to 900 μL 50% |
| | ACN in water to make third 10 X dilution (S3). |
| Step 4 | Take 5 μL of solution from S3 to 50 μL |
| | blank Plasma to make fourth 10X dilution (S4). |

Totally dose solution was diluted 100000×.

The concentrations of dose formulation were measured by LC-MS, and the results shown in the table below:

| | | Dilution | Nominal | Measured | Mean | Accuracy | SD | CV |
|---|---|---|---|---|---|---|---|---|
| Route | Sample Name | Factor | (mg/mL) | (mg/mL) | (mg/mL) | (%) | (mg/mL) | (%) |
| IN | IN_DOSE_1 | 100000 | 8.80 | 9.03 | 8.98 | 102 | 0.162 | 1.80 |
| | IN_DOSE_2 | 100000 | | 8.81 | | | | |
| | IN_DOSE_3 | 100000 | | 9.12 | | | | |
| PO | PO_DOSE_1 | 100000 | 0.880 | 0.871 | 0.901 | 102 | 0.0260 | 2.89 |
| | PO_DOSE_2 | 100000 | | 0.910 | | | | |
| | PO_DOSE_3 | 100000 | | 0.921 | | | | |

Sample Collection

For Group 1 (IN dose, 1.76 mg/kg), blood, CSF and brain samples were collected from 3 mice at two time points: 0.0833 h and 0.5 h post dose.

For Group 2 (PO dose, 10.56 mg/kg), blood, CSF and brain samples were collected from 3 mice at two time point: 0.5 h and 1 h post dose.

For plasma samples, about 200 μL of blood samples were collected from each animal via jugular vein. These blood samples were placed into the tubes containing K2EDTA, and centrifuged at 3000 g for 10 minutes in a 4° C. centrifuge. The completion time from collection was less than 30 min. The stabilizing agent of ascorbic acid at 200 mg/mL was added in a proportion of 10% in plasma. The plasma samples were stored in a freezer at −75±15° C. prior to analysis. The completion time from collection was less than 60 min.

For brain and CSF samples, The animals were fully exsanguinated with a rising concentration of CO2 gas prior to CSF and brain collection.

For CSF collection, an incision was made by scissors on the neck of the rats to expose the foramen magnum. A syringe fitted with a venoclysis needle was used to sample the CSF, the tip of the needle was inserted into the membrane of foramen magnum about 1-2 mm, and a negative pressure was applied by pulling a stopcock a small length of the syringe to extract CSF fluids into a tube. The stopcock was further actuated to until a sufficient amount of CSF was collected. There should be no blood in the CSF fluid. A stabilizing agent of ascorbic acid at 200 mg/mL was added in a proportion of 10% in CSF.

For brain collection. Brain samples were collected at an adopted time point, and washed 3 times with cold saline, then dried with clean gauze and put into the weighed tube, quick frozen in an ice box and kept at −75±15° C. All brain samples were weighted and homogenized with phosphate buffered saline (PBS) by brain weight (g) to PBS volume (mL) ratio 1:3 before analysis. The stabilizing agent of ascorbic acid at 200 mg/mL was added in a proportion of 10% in grain homogenate. The actual concentration was the detected value multiplied by the dilution factor. Brain samples were collected at designed time point, quickly homogenized and kept at −75±15° C.

### Preparation of Standards

About 1 mg (6aS)-6-methyl-5,6,6a,7-tetrahydro-4H-dibenzo[de,g]quinoline-10,11-diol hydrochloride hemihydrate and (6aR)-6-methyl-5,6,6a,7-tetrahydro-4H-dibenzo[de,g]quinoline-10,11-diol hydrochloride hemihydrate was dissolved in DMSO to obtain a 1 mg/mL standard stock solution (free base).

Calibration standard working solutions were prepared at concentrations of 2, 5, 10, 20, 50, 100, 500, 1000, 5000 and 10000 ng/mL by serial dilution of the standard stock solution in 50% acetonitrile in water. Quality control working solutions at concentrations of 5, 10, 20, 500 and 8000 ng/mL were prepared by serial dilution of the standard stock solution in 50% acetonitrile in water. These QC samples were prepared on the day of analysis in the same way as calibration standards.

Sample Treatment

For plasma, CSF, and brain samples, 5 μL of each calibration standard working solution (2, 5, 10, 50, 100, 500, 1000, 5000 and 10000 ng/mL) was added to 50 μL of the blank SD rat plasma, CSF or brain homogenate (with 10% 200 mg/mL ascorbic acid) to achieve calibration standards of 0.2-1000 ng/mL (0.2, 0.5, 1, 5, 10, 50, 100, 500, 1000 ng/mL) in a total volume of 55 μL. Quality Control (QC) samples at 0.5 ng/mL (low-1), 1 ng/mL (low-2), 50 ng/mL (mid), 800 ng/mL (high) for plasma, CSF or brain homogenate were prepared independently for those used for the calibration curves. These QC samples were prepared on the day of analysis in the same way as calibration standards. The total volumes of 55 μL of standards, 55 μL of QC samples and 55 μL of unknown samples (50 μL of unknown plasma, CSF or brain homogenate (with 10% 200 mg/mL ascorbic acid) with 5 μL 50% acetonitrile) were added to 200 μL of acetonitrile containing IS to precipitate proteins. Then the samples were vortexed for 30 sec. After centrifugation at 4° C., 4700 rpm for 30 min, and supernatant was diluted 2 times with water, 10 μL of the diluted supernatant was injected into the LC-MS/MS system for quantitative analysis.

All the samples were processed on ice.

LC-MS/MS Conditions

The LC-MS/MS system consisted of two Shimadzu LC-30AD pumps, a DGU-20A5R degasser, a Rack changer II and LCMS-8060 LC/MS/MS instrument mass spectrometer.

Chromatographic separation was performed on a DAICEL CORPORATION CHIRALCEL OD-3R Particle size 3 μm 4.6 mm×150 mm column at room temperature. The mobile phase was composed of A: Water (5 mM Ammonium Acetate); B: Ethanol (5 mM Ammonium Acetate). The flow rate was 0.7 mL/min. The injection volume was 10 μL.

Positive mode electrospray ionization (ESI) was performed to obtain a protonated ion of (6aR)-6-methyl-5,6,6a,7-tetrahydro-4H-dibenzo[de,g]quinoline-10,11-diol, (6aS)-6-methyl-5,6,6a,7-tetrahydro-4H-dibenzo[de,g]quinoline-10,11-diol, and Dexamethasone (IS). A multiple reaction monitoring (MRM) method was selected for quantitative analysis. The optimized transitions were 268.20191.10, 268.20191.10 and 393.20-373.20 for (6aR)-6-methyl-5,6,6a,7-tetrahydro-4H-dibenzo[de,g]quinoline-10,11-diol, (6aS)-6-methyl-5,6,6a,7-tetrahydro-4H-dibenzo[de,g]quinoline-10,11-diol, and Dexamethasone, respectively. The instrument parameters were set as follows: ion spray voltage: 8060; heating gas flow: 10 L/min; interface temperature: 250° C.; DL temperature: 220° C.; heat block temperature: 400° C.; drying gas flow: 10 L/min. (6aR)-6-methyl-5,6,6a,7-tetrahydro-4H-dibenzo[de,g]quinoline-10,11-diol, (6aS)-6-methyl-5,6,6a,7-tetrahydro-4H-dibenzo[de,g]quinoline-10,11-diol, and Dexamethasone dependent parameters are listed as follows:

| | (6aR)-6-methyl- | (6aS)-6-methyl- | |
|---|---|---|---|
| | 5,6,6a,7- | 5,6,6a,7-tetrahydro- | |
| | tetrahydro-4H- | 4H- | |
| | dibenzo[de,g]quin | dibenzo[de,g]quinolin | |
| Compound ID | oline-10,11-diol | e−10,11-diol | Dexamethasone (IS) |
| Transition | 268.20→191.10 | 268.20→191.10 | 393.20→373.20 |
| Dwell | 100 | 100 | 50 |
| Time(msec) | | | |
| Q1 Pre Bias (v) | −10 | −10 | −25 |
| CE | −30 | −30 | −10 |
| Q3 Pre Bias (v) | −19 | −19 | −18 |

Data acquisition was performed by Lab Solutions version 5.89 Software (Shimadzu). All concentration data was reported with 3 significant figures. Data statistics were performed using Excel 2010 software. The pharmacokinetic parameters of (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol were calculated using a non-compartmental approach with Phoenix™ WinNonlin®.

Results

The plasma, CSF, and brain concentrations for (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol in each animal for IN and PO dose at 1.76 mg/kg and 10.56 mg/kg, were used to calculate the ratio between brain or CSF concentrations and plasma concentrations.

The resulting of the ratio of 6aS)-6-methyl-5,6,6a,7-tetrahydro-4H-dibenzo[de,g]quinoline-10,11-diol between plasma and brain following IN administration to Male SD Rats at 1.76 mg/kg:

| | | | | Ratio (Brain/ | Mean Ratio |
|---|---|---|---|---|---|
| | | Brain | Plasma | Plasma) | (Brain/Plasma) |
| Time (h) | Animal | (ng/g) | (ng/mL) | (mL/g) | (mL/g) |
| 0.0833 | Rat 1 | 1029 | 460 | 2.24 | 3.78 |
| | Rat 2 | 1995 | 377 | 5.30 | |
| | Rat 3 | 1764 | 463 | 3.81 | |
| 0.5 | Rat 4 | 299 | 50.2 | 5.95 | 5.62 |
| | Rat 5 | 180 | 33.9 | 5.31 | |
| | Rat 6 | 441 | 78.9 | 5.59 | |

The resulting ratio of (6aS)-6-methyl-5,6,6a,7-tetrahydro-4H-dibenzo[de,g]quinoline-10,11-diol between plasma and CSF following IN administration to Male SD Rats at 1.76 mg/kg:

| | | CSF | Plasma | Ratio (CSF/ | Mean Ratio |
|---|---|---|---|---|---|
| Time (h) | Animal | (ng/mL) | (ng/mL) | Plasma) | (CSF/ Plasma) |
| 0.0833 | Rat 1 | 43.0 | 460 | 0.0935 | 0.137 |
| | Rat 2 | 70.0 | 377 | 0.186 | |
| | Rat 3 | 60.4 | 463 | 0.130 | |
| 0.5 | Rat 4 | 11.7 | 50.2 | 0.233 | 0.244 |
| | Rat 5 | 8.05 | 33.9 | 0.237 | |
| | Rat 6 | 20.7 | 78.9 | 0.263 | |

The resulting ratio of (6aS)-6-methyl-5,6,6a,7-tetrahydro-4H-dibenzo[de,g]quinoline-10,11-diol between plasma and brain following PO administration to Male SD Rats at 10.56 mg/kg:

| | | | | | Mean Ratio |
|---|---|---|---|---|---|
| | | Brain | Plasma | Ratio (Brain/ | (Brain/Plasma) |
| Time (h) | Animal | (ng/g) | (ng/mL) | Plasma) (mL/g) | (mL/g) |
| 0.5 | Rat 7 | 32.2 | 49.3 | 0.654 | 0.689 |
| | Rat 8 | 39.8 | 44.9 | 0.886 | |
| | Rat 9 | 18.8 | 35.6 | 0.528 | |
| 1 | Rat 10 | 17.1 | 28.9 | 0.590 | 0.391 |
| | Rat 11 | 12.9 | 43.6 | 0.297 | |
| | Rat 12 | 13.6 | 47.8 | 0.285 | |

The resulting ratio of (6aS)-6-methyl-5,6,6a,7-tetrahydro-4H-dibenzo[de,g]quinoline-10,11-diol between plasma and CSF following PO administration to Male SD Rats at 10.56 mg/kg:

| | | CSF | Plasma | Ratio (CSF/ | Mean Ratio |
|---|---|---|---|---|---|
| Time (h) | Animal | (ng/mL) | (ng/mL) | Plasma) | (CSF/ Plasma) |
| 0.5 | Rat 7 | 1.43 | 49.3 | 0.0290 | 0.0419 |
| | Rat 8 | 2.46 | 44.9 | 0.0547 | |
| | Rat 9 | BLOQ | 35.6 | NA | |
| 1 | Rat 10 | BLOQ | 28.9 | NA | NA |
| | Rat 11 | BLOQ | 43.6 | NA | |
| | Rat 12 | BLOQ | 47.8 | NA | |

The resulting ratios of (6aR)-6-methyl-5,6,6a,7-tetrahydro-4H-dibenzo[de,g]quinoline-10,11-diol between plasma and brain following IN administration to Male SD Rats at 1.76 mg/kg:

| | | | | Ratio (Brain/ | |
|---|---|---|---|---|---|
| | | Brain | Plasma | Plasma) | Mean Ratio (Brain/ |
| Time (h) | Animal | (ng/g) | (ng/mL) | (mL/g) | Plasma) (mL/g) |
| 0.0833 | Rat 1 | 18.5 | 4.00 | 4.62 | 7.16 |
| | Rat 2 | 31.8 | 3.38 | 9.43 | |
| | Rat 3 | 28.8 | 3.88 | 7.43 | |
| 0.5 | Rat 4 | 12.7 | BLOQ | NA | NA |
| | Rat 5 | 7.11 | BLOQ | NA | |
| | Rat 6 | 10.7 | 1.23 | 8.67 | |

Note:
NA = Not available, BLOQ = Below quantifiable limit of 1 ng/mL for plasma.

The resulting ratios of (6aR)-6-methyl-5,6,6a,7-tetrahydro-4H-dibenzo[de,g]quinoline-10,11-diol between plasma and CSF following IN administration to Male SD Rats at 1.76 mg/kg:

| | | CSF | Plasma | Ratio (CSF/ | Mean Ratio |
|---|---|---|---|---|---|
| Time (h) | Animal | (ng/mL) | (ng/mL) | Plasma) | (CSF/ Plasma) |
| 0.0833 | Rat 1 | BLOQ | 4.00 | NA | NA |
| | Rat 2 | 1.11 | 3.38 | 0.330 | |
| | Rat 3 | BLOQ | 3.88 | NA | |
| 0.5 | Rat 4 | BLOQ | BLOQ | NA | NA |
| | Rat 5 | BLOQ | BLOQ | NA | |
| | Rat 6 | BLOQ | 1.23 | NA | |

Note:
NA = Not available, BLOQ = Below quantifiable limit of 1 ng/mL for CSF and plasma.

The resulting ratios of (6aR)-6-methyl-5,6,6a,7-tetrahydro-4H-dibenzo[de,g]quinoline-10,11-diol between Plasma and Brain Following PO Administration to Male SD Rats at 10.56 mg/kg:

| | | | | | Mean Ratio |
|---|---|---|---|---|---|
| | | Brain | Plasma | Ratio (Brain/ | (Brain/ Plasma) |
| Time (h) | Animal | (ng/g) | (ng/mL) | Plasma) (mL/g) | (mL/g) |
| 0.5 | Rat 7 | 2.46 | BLOQ | NA | NA |
| | Rat 8 | 2.73 | BLOQ | NA | |
| | Rat 9 | 3.10 | BLOQ | NA | |
| 1 | Rat 10 | BLOQ | BLOQ | NA | NA |
| | Rat 11 | BLOQ | BLOQ | NA | |
| | Rat 12 | BLOQ | BLOQ | NA | |

Note:
NA = Not available, BLOQ = Below quantifiable limit of 1 ng/mL for plasma and 0.5 ng/mL for brain.

The resulting ratios of (6aR)-6-methyl-5,6,6a,7-tetrahydro-4H-dibenzo[de,g]quinoline-10,11-diol between Plasma and CSF Following PO Administration to Male SD Rats at 10.56 mg/kg:

| | | CSF | Plasma | Ratio (CSF/ | Mean Ratio |
|---|---|---|---|---|---|
| Time (h) | Animal | (ng/mL) | (ng/mL) | Plasma) | (CSF/Plasma) |
| 0.5 | Rat 7 | BLOQ | BLOQ | NA | NA |
| | Rat 8 | BLOQ | BLOQ | NA | |
| | Rat 9 | BLOQ | BLOQ | NA | |
| 1 | Rat 10 | BLOQ | BLOQ | NA | NA |
| | Rat 11 | BLOQ | BLOQ | NA | |
| | Rat 12 | BLOQ | BLOQ | NA | |

(6aS)-6-methyl-5,6,6a,7-tetrahydro-4H-dibenzo[de,g]quinoline-10,11-diol plasma concentrations following IN administration to male SD Rats at 1.76 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.0833 | 460 | 377 | 463 | 433 | 48.9 | 11.3 |
| 0.5 | 50.2 | 33.9 | 78.9 | 54.4 | 22.8 | 42.0 |

(6aS)-6-methyl-5,6,6a,7-tetrahydro-4H-dibenzo[de,g]quinoline-10,11-diol CSF concentrations following IN administration to male SD Rats at 1.76 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.0833 | 43.0 | 70.0 | 60.4 | 57.8 | 13.7 | 23.7 |
| 0.5 | 11.7 | 8.05 | 20.7 | 13.5 | 6.54 | 48.5 |

(6aS)-6-methyl-5,6,6a,7-tetrahydro-4H-dibenzo[de,g]quinoline-10,11-diol brain concentrations following IN administration to male SD Rats at 1.76 mg/kg:

| Time | Conc. (ng/mL) | | | Conc. (ng/g) | | | Mean | SD | CV |
|---|---|---|---|---|---|---|---|---|---|
| (h) | Sort 1 | Sort 2 | Sort 3 | Sort 1 | Sort 2 | Sort 3 | (ng/g) | (ng/g) | (%) |
| 0.0833 | 257 | 499 | 441 | 1029 | 1995 | 1764 | 1596 | 505 | 31.6 |
| 0.5 | 74.7 | 45.1 | 110 | 299 | 180 | 441 | 307 | 131 | 42.6 |

Note:
All the brain samples were added with PBS by brain weight (g) to PBS volume (mL) Ratio 1:3 for homogenizing. The actual concentration (ng/g) is the detected value (ng/mL) multiplied by 4.

(6aS)-6-methyl-5,6,6a,7-tetrahydro-4H-dibenzo[de,g]quinoline-10,11-diol plasma concentrations following PO administration to male SD Rats at 10.56 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.5 | 49.3 | 44.9 | 35.6 | 43.3 | 7.03 | 16.2 |
| 1 | 28.9 | 43.6 | 47.8 | 40.1 | 9.90 | 24.7 |

(6aS)-6-methyl-5,6,6a,7-tetrahydro-4H-dibenzo[de,g]quinoline-10,11-diol CSF concentrations following PO administration to male SD Rats at 10.56 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.5 | 1.43 | 2.46 | BLOQ | 1.95 | NA | NA |
| 1 | BLOQ | BLOQ | BLOQ | NA | NA | NA |

Note:
NA = Not available,
BLOQ = Below quantifiable limit of 1 ng/mL for CSF.

(6aS)-6-methyl-5,6,6a,7-tetrahydro-4H-dibenzo[de,g]quinoline-10,11-diol brain concentrations following PO administration to Male SD Rats at 10.56 mg/kg:

| Time | Conc. (ng/mL) | | | Conc. (ng/g) | | | Mean | SD | CV |
|---|---|---|---|---|---|---|---|---|---|
| (h) | Sort 1 | Sort 2 | Sort 3 | Sort 1 | Sort 2 | Sort 3 | (ng/g) | (ng/g) | (%) |
| 0.5 | 8.06 | 9.95 | 4.70 | 32.2 | 39.8 | 18.8 | 30.3 | 10.7 | 35.2 |
| 1 | 4.27 | 3.24 | 3.40 | 17.1 | 12.9 | 13.6 | 14.5 | 2.22 | 15.2 |

Note:
All the brain samples were added with PBS by brain weight (g) to PBS volume (mL) Ratio 1:3 for homogenizing. The actual concentration (ng/g) is the detected value (ng/mL) multiplied by 4.

(6aR)-6-methyl-5,6,6a,7-tetrahydro-4H-dibenzo[de,g]quinoline-10,11-diol plasma concentrations following IN administration to male SD Rats at 1.76 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.0833 | 4.00 | 3.38 | 3.88 | 3.75 | 0.331 | 8.82 |
| 0.5 | BLOQ | BLOQ | 1.23 | NA | NA | NA |

Note:
NA = Not available,
BLOQ = Below quantifiable limit of 1 ng/mL for plasma.

(6aR)-6-methyl-5,6,6a,7-tetrahydro-4H-dibenzo[de,g]quinoline-10,11-diol CSF concentrations following IN administration to male SD Rats at 1.76 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.0833 | BLOQ | 1.11 | BLOQ | NA | NA | NA |
| 0.5 | BLOQ | BLOQ | BLOQ | NA | NA | NA |

Note:
NA = Not available,
BLOQ = Below quantifiable limit of 1 ng/mL for CSF.

(6aR)-6-methyl-5,6,6a,7-tetrahydro-4H-dibenzo[de,g]quinoline-10,11-diol brain concentrations following IN administration to male SD Rats at 1.76 mg/kg:

| Time | Conc. (ng/mL) | | | Conc. (ng/g) | | | Mean | SD | CV |
|---|---|---|---|---|---|---|---|---|---|
| (h) | Sort 1 | Sort 2 | Sort 3 | Sort 1 | Sort 2 | Sort 3 | (ng/g) | (ng/g) | (%) |
| 0.0833 | 4.62 | 7.96 | 7.20 | 18.5 | 31.8 | 28.8 | 26.4 | 6.99 | 26.5 |
| 0.5 | 3.19 | 1.78 | 2.68 | 12.7 | 7.11 | 10.7 | 10.2 | 2.85 | 28.0 |

Note:
All the brain samples were added with PBS by brain weight (g) to PBS volume (mL) Ratio 1:3 for homogenizing. The actual concentration (ng/g) is the detected value (ng/mL) multiplied by 4.

(6aR)-6-methyl-5,6,6a,7-tetrahydro-4H-dibenzo[de,g]quinoline-10,11-diol plasma concentrations following PO administration to male SD Rats at 10.56 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.5 | BLOQ | BLOQ | BLOQ | NA | NA | NA |
| 1 | BLOQ | BLOQ | BLOQ | NA | NA | NA |

Note:
NA = Not available,
BLOQ = Below quantifiable limit of 1 ng/mL for plasma.

(6aR)-6-methyl-5,6,6a,7-tetrahydro-4H-dibenzo[de,g]quinoline-10,11-diol CSF concentrations following PO administration to male SD Rats at 10.56 mg/kg:

| Time | Conc. (ng/mL) | | | Mean | SD | CV |
|---|---|---|---|---|---|---|
| (h) | Sort 1 | Sort 2 | Sort 3 | (ng/mL) | (ng/mL) | (%) |
| 0.5 | BLOQ | BLOQ | BLOQ | NA | NA | NA |
| 1 | BLOQ | BLOQ | BLOQ | NA | NA | NA |

Note:
NA = Not available,
BLOQ = Below quantifiable limit of 1 ng/mL for CSF.

(6aR)-6-methyl-5,6,6a,7-tetrahydro-4H-dibenzo[de,g]quinoline-10,11-diol brain concentrations following PO administration to Male SD Rats at 10.56 mg/kg:

| Time | Conc. (ng/mL) | | | Conc. (ng/g) | | | Mean | SD | CV |
|---|---|---|---|---|---|---|---|---|---|
| (h) | Sort 1 | Sort 2 | Sort 3 | Sort 1 | Sort 2 | Sort 3 | (ng/g) | (ng/g) | (%) |
| 0.5 | 0.616 | 0.683 | 0.775 | 2.46 | 2.73 | 3.10 | 2.77 | 0.319 | 11.5 |
| 1 | BLOQ | BLOQ | BLOQ | NA | NA | NA | NA | NA | NA |

Note:
NA = Not available, BLOQ = Below quantifiable limit of 0.5 ng/mL for brain. All the brain samples were added with PBS by brain weight (g) to PBS volume (mL) Ratio 1:3 for homogenizing. The actual concentration (ng/g) is the detected value (ng/mL) multiplied by 4.

### Example 5: Plasma and CSF Pharmacokinetic Study of (6aS)-6-methyl-5,6,6a,7-tetrahydro-4H-dibenzo[de,g]quinoline-10,11-diol Following Single Sublingual, Oral

Administration or Subcutaneous Injection of (6aS)-6-methyl-5,6,6a,7-tetrahydro-4H-dibenzo[de,g]quinoline-10,11-diol to Non-Naïve Male Beagle Dogs

The pharmacokinetics of (6aS)-6-methyl-5,6,6a,7-tetrahydro-4H-dibenzo[de,g]quinoline-10,11-diol were evaluated in 9 male non-naïve beagle dogs following single sublingual (SL) administration at 5 mg/kg, oral (PO) administration at 10 mg/kg, and subcutaneous (SC) injection at 1 mg/kg.

Animals

Nine non-naïve male beagle dogs supplied by Marshall Bioresources (Beijing, China) were used in this study. The animals were confirmed healthy before being assigned to the study. The room(s) were controlled and monitored for relative humidity (targeted mean range 40% to 70%, and were on a 12-hour light/dark cycle except when interruptions are necessitated by study activities.

Animals were fed twice daily. Stock Dogs were fed approximately 220 grams of Certified Dog Diet daily (Beijing Keao Xieli Feed Co., Ltd. Beijing, P. R. China). These amounts could be adjusted as necessary based on food consumption of the group or an individual body weight change. For SC, SL and PO groups, animals were fed at 3:30 to 4:00 pm on the day prior to oral dosing and any remaining food was removed at 7:00 pm. Food was withheld until 4-hour post-dose. Animals were fed once on the day of dosing, with the amount of approximately 220 grams.

Study Design

Nine male non-naïve beagle dogs were assigned to this study consisting of three groups. In group 1, animals were treated with (6aS)-6-methyl-5,6,6a,7-tetrahydro-4H-dibenzo[de,g]quinoline-10,11-diol by single SL administration at 5 mg/kg, which was formulated in 0.83% HPMC (40˜60 cp):PEG400:VE-TPGS=60:30:10. In group 2, animals were treated with (6aS)-6-methyl-5,6,6a,7-tetrahydro-4H-dibenzo[de,g]quinoline-10,11-diol by PO administration at 10 mg/kg, which was formulated in 0.5% CMC-Na in water. In group 3, animals were treated with (6aS)-6-methyl-5,6,6a,7-tetrahydro-4H-dibenzo[de,g]quinoline-10,11-diol by SC administration at 1 mg/kg, which was formulated in saline. The dosing regimens and formulation (vehicle) compositions are described in the following table below:

| | | | Dose | | | | |
|---|---|---|---|---|---|---|---|
| | | Dose Level | Volume | Conc. | | | No. of |
| Group | Treatment | (mg/kg) | (mL/kg) | (mg/mL) | Vehicle | Route | Animals |
| G1 | (6aS)-6-methyl- | 5 | 0.1 | 50 | 0.83% HPMC | SL (fasted) | 3 (D1001, |
| | 5,6,6a,7- | | | | (40~60 | | D1002, |
| | tetrahydro-4H- | | | | cp):PEG400:VE- | | D1003) |
| | dibenzo[de,g]quin- | | | | TPGS = 60:30:10 | | |
| | oline-10,11- | | | | | | |
| | diol | | | | | | |
| | hydrochloride | | | | | | |
| G2 | (6aS)-6-methyl- | 10 | 5 | 2 | 0.5% CMC- | PO (fasted) | 3 (D2001, |
| | 5,6,6a,7- | | | | Na in water | | D2002, |
| | tetrahydro-4H- | | | | | | D2003) |
| | dibenzo[de,g]quin- | | | | | | |
| | oline-10,11- | | | | | | |
| | diol | | | | | | |
| | hydrochloride | | | | | | |
| G3 | (6aS)-6-methyl- | 1 | 1 | 1 | Saline | SC (fasted) | 3 (D0001, |
| | 5,6,6a,7- | | | | | | D3002, |
| | tetrahydro-4H- | | | | | | D3003) |
| | dibenzo[de,g]quin- | | | | | | |
| | oline-10,11- | | | | | | |
| | diol | | | | | | |
| | hydrochloride | | | | | | |

The sampling regimens are described in the following table below:

| | Dose | | | | | | | | | | |
|---|---|---|---|---|---|---|---|---|---|---|---|
| | Level | | Sampling Time Point (hr) | | | | | | | | |
| Group | (mg/kg) | Pre-dose | 0.033 | 0.167 | 0.25 | 0.5 | 1 | 2 | 4 | 8 | 24 |
| G1P | 5 | P + CSF | P + CSF | P + CSF | P + CSF | P + CSF | P + CSF | P + CSF | P + CSF | P + CSF | P |
| G2P | 10 | P + CSF | P + CSF | P + CSF | P + CSF | P + CSF | P + CSF | P + CSF | P + CSF | P + CSF | P |
| G3P | 1 | P + CSF | P + CSF | P + CSF | P + CSF | P + CSF | P + CSF | P + CSF | P + CSF | P + CSF | P |

P = Plasma;
CSF = Cerebral Spinal Fluid

Dose Administration

Animals were weighed prior to dose administration on the day of dosing for calculating the actual dose volume. In group 1, animals were treated with (6aS)-6-methyl-5,6,6a,7-tetrahydro-4H-dibenzo[de,g]quinoline-10,11-diol by single SL administration at 5 mg/kg, which was formulated in 0.83% HPMC (40-60 cp):PEG400:VE-TPGS=60:30:10. In group 2, animals were treated with (6aS)-6-methyl-5,6,6a,7-tetrahydro-4H-dibenzo[de,g]quinoline-10,11-diol by PO administration at 10 mg/kg, which was formulated in 0.5% CMC-Na in water. In group 3, animals were treated with (6aS)-6-methyl-5,6,6a,7-tetrahydro-4H-dibenzo[de,g]quinoline-10,11-diol by SC administration at 1 mg/kg, which was formulated in Saline.

Sample Collection

Approximately 0.5 mL blood was collected at each time point. All blood samples were collected from a peripheral vessel. All blood samples were transferred into commercially available tubes containing Potassium (K2) EDTA on wet ice and processed for plasma. Samples were centrifuged (3,000×g for 10 minutes at 2 to 8° C.) within 30 minutes of collection. After centrifugation, the stabilizing agent was added to the harvested plasma within 60 minutes. The samples were kept on ice and protected from sunlight (under yellow light). The stabilizing agent was ascorbic acid 200 mg/mL added in a proportion of 10% in plasma. The plasma samples were divided into two equal samples with each at least 0.1 ml (0.1*2 mL/sample) and transferred into labeled polypropylene micro-centrifuge tubes and stored frozen in a freezer set to maintain −60° C. or lower until analyzed.

About 0.1 mL/time point (50 μL*2 aliquots) of CSF were collected via the catheter implanted in the Cisterna Magna of conscious beagle dogs without any sedation. For CSF collection, a polypropylene tube was first weighted to accuracy of 0.1 mg (weight #1). CSF was collected in the tube and weighted again (weight #2). Total CSF weight was calculated by subtracting weight #1 from weight #2. Samples were kept on ice and protected from sunlight. Stabilizing agent (ascorbic acid 200 mg/mL) was added within 60 minutes in a proportion of 10% in CSF. Frozen CSF samples on dry ice were stored at −60° C. or lower until bioanalysis.

### Preparation of Standards

In plasma, blank male Beagle dog plasma with EDTA-K2 as anti-coagulant was used for the preparation of standards. In CSF blank artificial CSF was used for the preparation of standards.

About 1.82 mg of (6aS)-6-methyl-5,6,6a,7-tetrahydro-4H-dibenzo[de,g]quinoline-10,11-diol was dissolved in DMSO to obtain a 1000 μg/mL stock solution (Stock A).

The preparation procedures of (6aS)-6-methyl-5,6,6a,7-tetrahydro-4H-dibenzo[de,g]quinoline-10,11-diol working solutions are detailed in the table below:

| Source Solution | | | Dilution | Working Solution | | |
|---|---|---|---|---|---|---|
| Source | Conc. | Volume | Volume | Volume | Final Conc. | Working |
| Solution ID | (μg/mL) | (μL) | (μL) | (μL) | (μg/mL) | Solution ID |
| Stock A | 1000 | 30 | 470 | 500 | 60 | WS-C1 |
| WS-C1 | 60 | 120 | 240 | 360 | 20 | WS-C2 |
| WS-C2 | 20 | 160 | 160 | 320 | 10 | WS-C3 |
| WS-C3 | 10 | 80 | 320 | 400 | 2 | WS-C4 |
| WS-C4 | 2 | 160 | 160 | 320 | 1 | WS-C5 |
| WS-C5 | 1 | 80 | 320 | 400 | 0.2 | WS-C6 |
| WS-C6 | 0.2 | 80 | 320 | 400 | 0.04 | WS-C7 |
| WS-C7 | 0.04 | 160 | 160 | 320 | 0.02 | WS-C8 |
| Stock A | 1000 | 30 | 595 | 625 | 48 | WS-QC1 |
| WS-QC1 | 48 | 120 | 240 | 360 | 16 | WS-QC2 |
| WS-QC2 | 16 | 20 | 380 | 400 | 0.8 | WS-QC3 |
| WS-QC3 | 0.8 | 30 | 370 | 400 | 0.06 | WS-QC4 |
| Stock A | 1000 | 30 | 270 | 300 | 100 | WS-DQC1 |

Dilution Solvent: ACN/water (v:v, 70:30)

Preparation details of the (6aS)-6-methyl-5,6,6a,7-tetrahydro-4H-dibenzo[de,g]quinoline-10,11-diol calibration and QC standards in plasma and CSF are shown in the following table:

| Working Solution | | | Matrix | Calibration Standard | | |
|---|---|---|---|---|---|---|
| Source | Conc. | Volume | Volume | Volume | Final Conc. | Calibration |
| Solution ID | (μg/mL) | (μL) | (μL) | (μL) | (ng/mL) | Standard ID |
| WS-C1 | 60 | 3 | 57 | 60 | 3000 | C1 |
| WS-C2 | 20 | 3 | 57 | 60 | 1000 | C2 |
| WS-C3 | 10 | 3 | 57 | 60 | 500 | C3 |
| WS-C4 | 2 | 3 | 57 | 60 | 100 | C4 |
| WS-C5 | 1 | 3 | 57 | 60 | 50 | C5 |
| WS-C6 | 0.2 | 3 | 57 | 60 | 10 | C6 |
| WS-C7 | 0.04 | 3 | 57 | 60 | 2 | C7 |
| WS-C8 | 0.02 | 3 | 57 | 60 | 1 | C8 |

| Source | Conc. | Volume | Volume | Volume | Final Conc. | |
|---|---|---|---|---|---|---|
| Solution ID | (μg/mL) | (μL) | (μL) | (μL) | (ng/mL) | QCID |
| WS-QC1 | 48 | 3 | 57 | 60 | 2400 | QC1 |
| WS-QC2 | 16 | 3 | 57 | 60 | 800 | QC2 |
| WS-QC3 | 0.8 | 3 | 57 | 60 | 40 | QC3 |
| WS-QC4 | 0.06 | 3 | 57 | 60 | 3 | QC4 |
| WS-DQC1 | 100 | 3 | 57 | 60 | 5000 | DQC |

The concentrations of calibration and QC samples are shown in the table below:

| | | Calibration Standard | Quality Control |
|---|---|---|---|
| | | Samples Cone. | Samples Cone. |
| Compound | Matrix | (ng/mL) | (ng/mL) |
| (6aS)-6-methyl- | Male Beagle dog | 1, 2, 10, 50, 500, 3000 | 3, 40, 800, 2400, |
| 5,6,6a,7-tetrahydro-4H- | plasma | | 5000(DQC) |
| dibenzo [de,g]quinoline- | | | |
| 10,11-diol group 1 | | | |
| (6aS)-6-methyl- | Male Beagle dog | 1, 2, 10, 50, 100, 500, | 3, 40, 800, 2400 |
| 5,6,6a,7-tetrahydro-4H- | plasma | 1000, 3000 | |
| dibenzo [de,g]quinoline- | | | |
| 10,11-diol groups 2 and | | | |
| 3 | | | |
| (6aS)-6-methyl- | Male Beagle dog CSF | 1, 2, 10, 50, 100, 500, | 3, 40, 800, 2400 |
| 5,6,6a,7-tetrahydro-4H- | | 1000, 3000 | |
| dibenzo [de,g]quinoline- | | | |
| 10,11-diol group 1 | | | |
| (6aS)-6-methyl- | Male Beagle dog CSF | 1, 10, 50, 100, 500, | |
| 5,6,6a,7-tetrahydro-4H- | | 1000, 3000 | |
| dibenzo [de,g]quinoline- | | | |
| 10,11-diol group 1 | | | |
| retest | | | |
| (6aS)-6-methyl- | Male Beagle dog CSF | 1, 2, 10, 100, 500, 1000, | 3, 40, 800, 2400 |
| 5,6,6a,7-tetrahydro-4H- | | 3000 | |
| dibenzo [de,g]quinoline- | | | |
| 10,11-diol group 2 | | | |
| (6aS)-6-methyl- | Male Beagle dog CSF | 1, 2, 50, 100, 500, 1000, | 3, 40, 800, 2400 |
| 5,6,6a,7-tetrahydro-4H- | | 3000 | |
| dibenzo [de,g]quinoline- | | | |
| 10,11-diol group 3 | | | |

Sample Treatment

All CSF samples were added with the equal volume of plasma. An aliquot of 10 μL sample with 200 μL internal standard (100 ng/mL Labetalol & 100 ng/mL Dexamethasone & 100 ng/mL Tolbutamide & 100 ng/mL Verapamil & 100 ng/mL Glyburide & 200 ng/mL Dclofenac in ACN), the mixture was vortex-mixed well and centrifuged at 4000 rpm for 15 min, 4° C. 30 μL supernatant was then mixed with 30 μL water, vortex-mixed well and centrifuged at 4° C. 3 μL sample was injected for LC-MS/MS analysis.

Dilution procedure description: Dilution factor as 5—An aliquot of 2 μL sample was added with 8 μL blank matrix.

LC-MS/MS Conditions

The LC-MS/MS system consisted of an ACQUITY UPLC System and API 4000 mass spectrometer. Chromatographic separation was performed on a ACQUITY UPLC HSS T3 (2.1×50 mm, 1.8 μm) column. The mobile phase was composed of A: 0.1% formic acid and 2 mM HCOONH₄ in Water:ACN (v:v, 95:5); and B: 0.1% formic acid & 2 mM HCOONH₄ in ACN:Water (v:v, 95:5). The flow rate was 0.6 mL/min. The injection volume was 3 μL. The following gradient was implemented:

| | Flow Rate | | |
|---|---|---|---|
| Time (min) | (mL/min) | A (%) | B (%) |
| Initial | 0.600 | 100 | 0 |
| 0.30 | 0.600 | 100 | 0 |
| 1.10 | 0.600 | 5 | 95 |
| 1.40 | 0.600 | 5 | 95 |
| 1.41 | 0.600 | 100 | 0 |
| 1.60 | 0.600 | 100 | 0 |

Positive mode electrospray ionization (ESI) was performed to obtain a protonated ion of (6aS)-6-methyl-5,6,6a,7-tetrahydro-4H-dibenzo[de,g]quinoline-10,11-diol, and Diclofenac (IS). A multiple reaction monitoring (MRM) method was selected for quantitative analysis. The (6aS)-6-methyl-5,6,6a,7-tetrahydro-4H-dibenzo[de,g]quinoline-10,11-diol and Diclofenac dependent parameters are listed as follows:

| | | Collision Energy | Retention |
|---|---|---|---|
| Compound Name | Ion Transition | (eV) | Time (min) |
| (6aS)-6-methyl- | 268.0/219.0 | 32 | 0.85 |
| 5,6,6a,7-tetrahydro- | | | |
| 4H- | | | |
| dibenzo [de,g] quinoline- | | | |
| 10,11-diol | | | |
| Diclofenac | 296.0/214.0 | 40 | 1.29 |

Data acquisition was performed by Analyst 1.6.2 software was used for processing the data of all samples.

Results

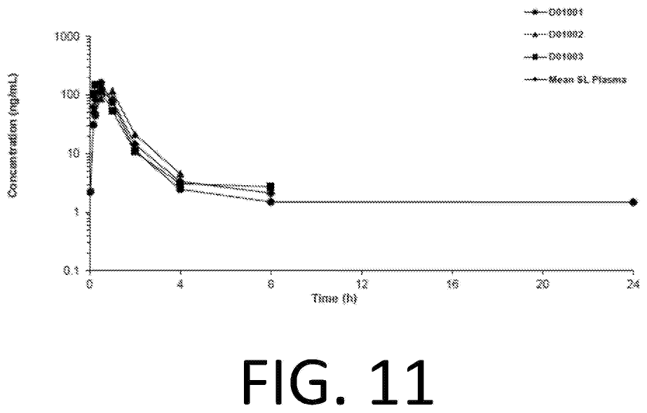

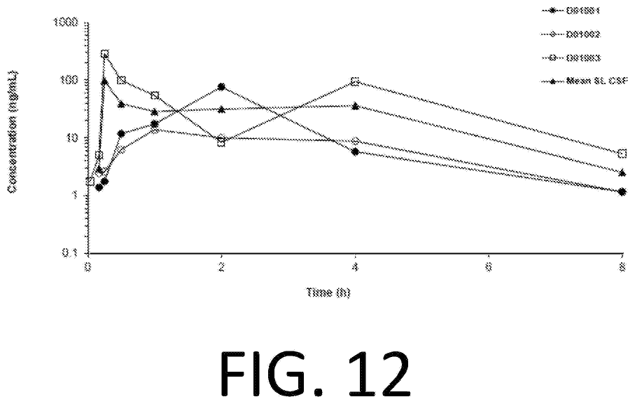

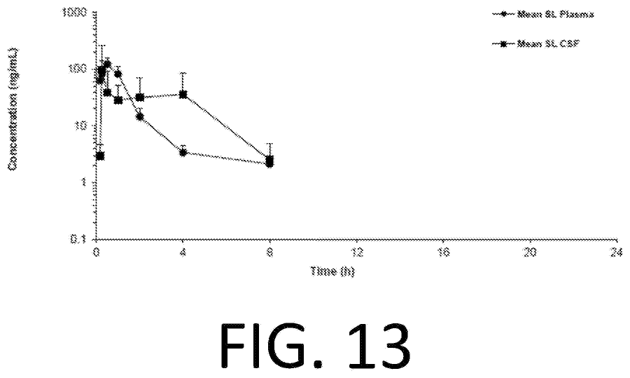

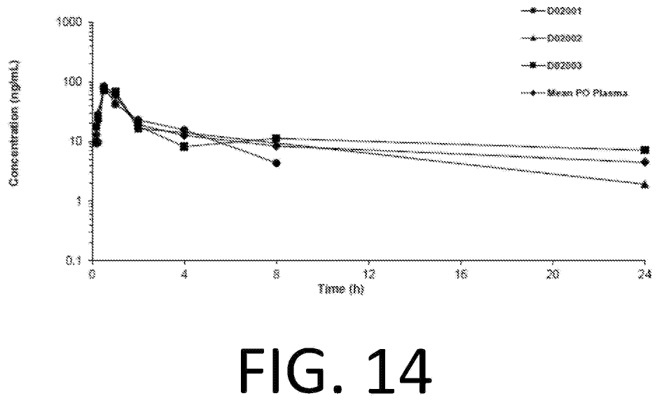

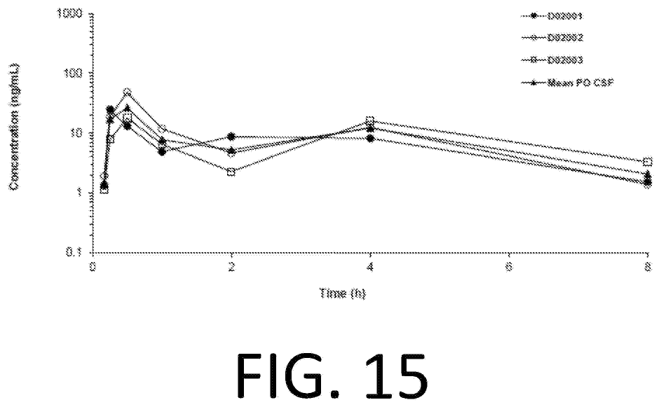

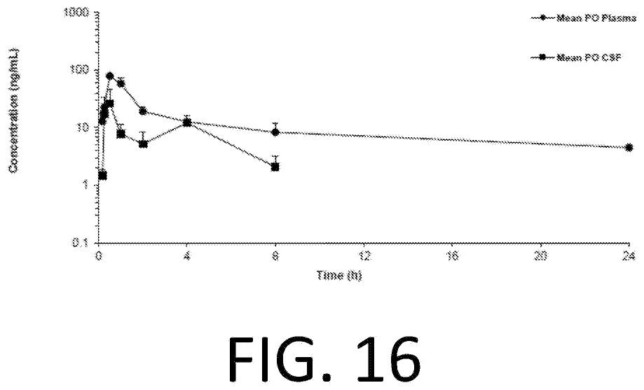

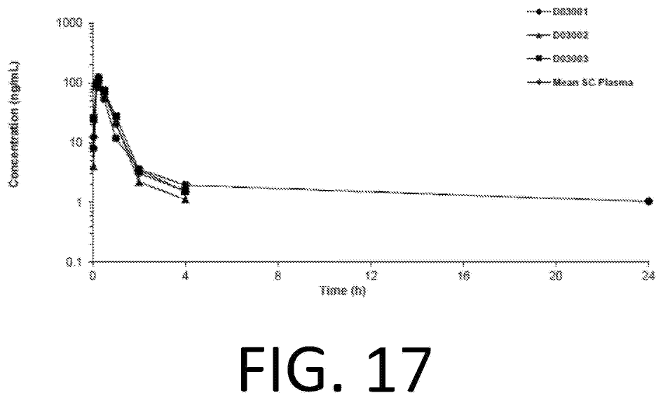

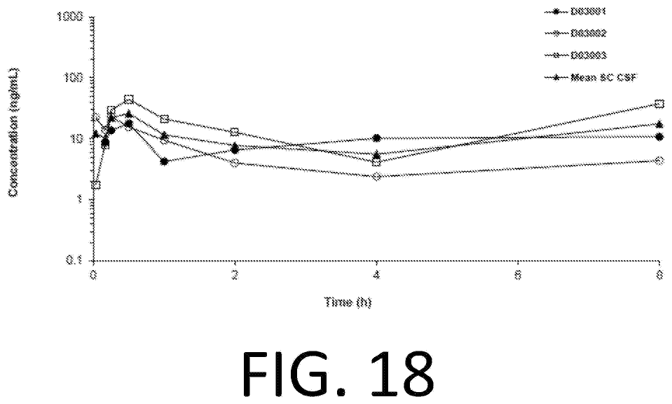

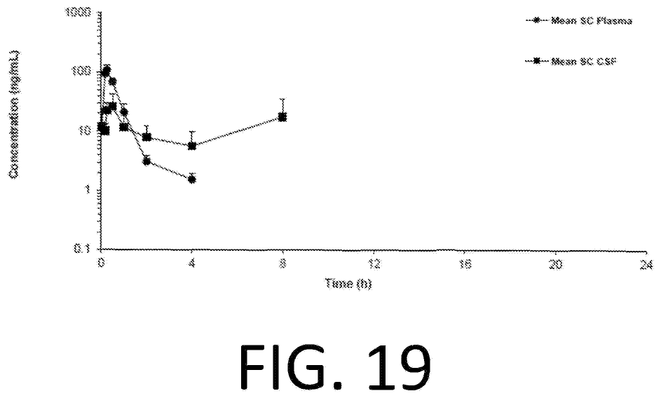

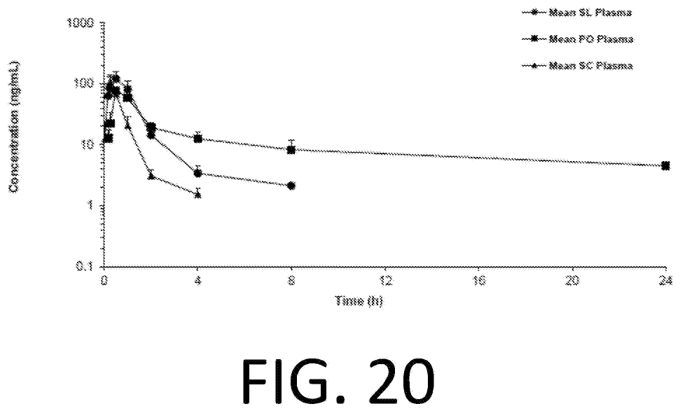

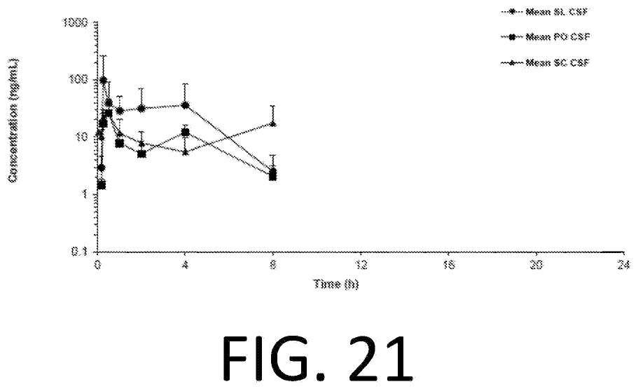

The individual and mean plasma concentration-time profiles are illustrated from FIGS. 11-21.

In group 1: For the SL dose at 5 mg/kg, (6aS)-6-methyl-5,6,6a,7-tetrahydro-4H-dibenzo[de,g]quinoline-10,11-diol showed a maximum plasma concentration (Cₘₐₓ) of 143±24.0 ng/mL, with time to reach Cₘₐₓ (Tₘₐₓ) at 0.583±0.382 h. (6aS)-6-methyl-5,6,6a,7-tetrahydro-4H-dibenzo[de,g]quinoline-10,11-diol showed a maximum CSF concentration (Cₘₐₓ) of 126±144 ng/mL, with time to reach Cₘₐₓ (Tₘₐₓ) at 1.08±0.878 h. The half-life (t_{1/2}) of (6aS)-6-methyl-5,6,6a,7-tetrahydro-4H-dibenzo[de,g]quinoline-10,11-diol in plasma and CSF were 2.76±2.95 h and 1.79±0.705 h, respectively. The AUCₗₐₛₜ and AUCinf values in plasma were 155±10.5 ng·h/mL and 162±14.5 ng·h/mL, respectively. The AUCₗₐₛₜ and AUCinf values in CSF were 162±135 ng·h/mL and 170±144 ng·h/mL, respectively. The AUCₗₐₛₜ ratio of CSF to total plasma was 1.08±0.964.

The table below demonstrates the individual and mean plasma PK parameters of (6aS)-6-methyl-5,6,6a,7-tetrahydro-4H-dibenzo[de,g]quinoline-10,11-diol in male Beagle dogs following single SL administration of (6aS)-6-methyl-5,6,6a,7-tetrahydro-4H-dibenzo[de,g]quinoline-10,11-diol at 5 mg/kg:

| PK | | | | Mean SL | | CV |
|---|---|---|---|---|---|---|
| Parameters | D01001 | D01002 | D01003 | Plasma | SD | (%) |
| No. points used for t_{1/2} | 5.00 | 3.00 | 5.00 | ND | — | — |
| Cₘₐₓ (ng/mL) | 162 | 116 | 151 | 143 | 24.0 | 16.8 |
| Tₘₐₓ (h) | 0.500 | 1.00 | 0.250 | 0.583 | 0.382 | 65.5 |
| t_{1/2} (h) | 6.13 | 0.671 | 1.48 | 2.76 | 2.95 | 107 |
| Tₗₐₛₜ (h) | 24.0 | 4.00 | 8.00 | ND | — | — |
| AUCₗₐₛₜ | 165 | 154 | 144 | 155 | 10.5 | 6.79 |
| (ng · h/mL) | | | | | | |
| AUC_{inf} | 178 | 159 | 150 | 162 | 14.5 | 8.92 |
| (ng · h/mL) | | | | | | |
| MRTₗₐₛₜ (h) | 3.39 | 1.19 | 1.29 | 1.96 | 1.24 | 63.3 |
| MRT_{inf} (h) | 5.57 | 1.30 | 1.64 | 2.83 | 2.37 | 83.7 |
| AUC_{Extra} (%) | 7.39 | 2.79 | 3.90 | 4.69 | 2.40 | 51.1 |
| AUMC_{Extra} | 43.6 | 10.7 | 24.2 | 26.1 | 16.5 | 63.3 |
| (%) | | | | | | |
| AUCₗₐₛₜ/Cₘₐₓ | 1.019 | 1.328 | 0.954 | 1.100 | 0.200 | 18.2 |
| (h) | | | | | | |

“—” means not applicable
“ND” means not determined
“D#” means animal number

The following table illustrates the individual mean CSF PK parameters of (6aS)-6-methyl-5,6,6a,7-tetrahydro-4H-dibenzo[de,g]quinoline-10,11-diol in male Beagle dogs following single SL administration of (6aS)-6-methyl-5,6,6a,7-tetrahydro-4H-dibenzo[de,g]quinoline-10,11-diol at 5 mg/kg:

| PK | | | | Mean SL | | CV |
|---|---|---|---|---|---|---|
| Parameters | D01001 | D01002 | D01003 | CSF | SD | (%) |
| No. points used for t_{1/2} | 3.00 | 3.00 | 5.00 | ND | — | — |
| Cₘₐₓ (ng/mL) | 76.2 | 13.8 | 289 | 126 | 144 | 114 |
| Tₘₐₓ (h) | 2.00 | 1.00 | 0.250 | 1.08 | 0.878 | 81.0 |
| t_{1/2} (h) | 1.06 | 1.83 | 2.47 | 1.79 | 0.705 | 39.5 |
| Tlast (h) | 8.00 | 8.00 | 8.00 | 8.00 | — | — |
| AUCₗₐₛₜ | 122 | 52.0 | 313 | 162 | 135 | 83.2 |
| (ng · h/mL) | | | | | | |
| AUC_{inf} | 124 | 55.1 | 332 | 170 | 144 | 84.6 |
| (ng · h/mL) | | | | | | |
| MRTₗₐₛₜ (h) | 2.43 | 3.05 | 3.04 | 2.84 | 0.353 | 12.4 |
| MRTinf (h) | 2.54 | 3.47 | 3.53 | 3.18 | 0.555 | 17.5 |
| AUC_{Extra} (%) | 1.45 | 5.55 | 5.65 | 4.22 | 2.40 | 56.9 |
| AUMC_{Extra} | 5.44 | 17.0 | 18.5 | 13.7 | 7.16 | 52.4 |
| (%) | | | | | | |
| AUC Ratio | 0.738 | 0.337 | 2.17 | 1.08 | 0.964 | 89.1 |
| AUCₗₐₛₜ/Cₘₐₓ | 1.601 | 3.768 | 1.083 | 2.151 | 1.424 | 66.2 |
| (h) | | | | | | |

“—” means not applicable
“ND” means not determined
“D#” means animal number

In group 2: For the PO dose at 10 mg/kg, (6aS)-6-methyl-5,6,6a,7-tetrahydro-4H-dibenzo[de,g]quinoline-10,11-diol showed a plasma Cₘₐₓ of 78.4±6.21 ng/mL, with Tₘₐₓ at 0.500±0.000 h. (6aS)-6-methyl-5,6,6a,7-tetrahydro-4H-dibenzo[de,g]quinoline-10,11-diol showed a CSF Cₘₐₓ of 30.3±16.1 ng/mL, with Tₘₐₓ at 0.417±0.144 h. The t_{1/2} of (6aS)-6-methyl-5,6,6a,7-tetrahydro-4H-dibenzo[de,g]quinoline-10,11-diol in plasma and CSF were 11.3±11.5 h and 10.1±13.3 h, respectively. The AUCₗₐₛₜ and AUCinf values in plasma were 228±74.9 ng·h/mL and 322±199 ng·h/mL, respectively. The AUCₗₐₛₜ and AUC_{inf} values in CSF were 58.1±8.47 ng·h/mL and 102±68.9 ng·h/mL, respectively. The AUCₗₐₛₜ ratio of CSF to total plasma was 0.270±0.0663.

The table below demonstrates the individual and mean plasma PK parameters of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg:

| PK | | | | Mean PO | | CV |
|---|---|---|---|---|---|---|
| Parameters | D02001 | D02002 | D02003 | Plasma | SD | (%) |
| No. points used for t_{1/2} | 3.00 | 3.00 | 4.00 | ND | — | — |
| Cₘₐₓ (ng/mL) | 84.8 | 72.4 | 78.0 | 78.4 | 6.21 | 7.92 |
| Tₘₐₓ (h) | 0.500 | 0.500 | 0.500 | 0.500 | 0.000 | 0.0 |
| t_{1/2} (h) | 2.46 | 7.00 | 24.3 | 11.3 | 11.5 | 102 |
| Tₗₐₛₜ (h) | 8.00 | 24.0 | 24.0 | ND | — | — |
| AUCₗₐₛₜ | 149 | 236 | 298 | 228 | 74.9 | 32.9 |
| (ng · h/mL) | | | | | | |
| AUC_{inf} | 164 | 256 | 546 | 322 | 199 | 61.9 |
| (ng · h/mL) | | | | | | |
| MRTₗₐₛₜ (h) | 2.57 | 6.32 | 8.74 | 5.88 | 3.11 | 52.9 |
| MRT_{inf} (h) | 3.42 | 8.44 | 31.6 | 14.5 | 15.0 | 104 |
| AUC_{Extra} (%) | 9.43 | 7.63 | 45.4 | 20.8 | 21.3 | 102 |
| AUMC_{Extra} | 31.9 | 30.8 | 84.9 | 49.2 | 30.9 | 62.9 |
| (%) | | | | | | |
| AUCₗₐₛₜ/Cₘₐₓ | 1.757 | 3.260 | 3.821 | 2.946 | 1.067 | 36.2 |
| (h) | | | | | | |

“—” means not applicable
“ND” means not determined
“D#” means animal number

The following table illustrates the individual mean CSF PK parameters of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg:

| PK | | | | Mean PO | | CV |
|---|---|---|---|---|---|---|
| Parameters | D02001 | D02002 | D02003 | CSF | SD | (%) |
| No. points used for t_{1/2} | 3.00 | 4.00 | 4.00 | ND | — | — |
| Cₘₐₓ (ng/mL) | 24.7 | 48.5 | 17.8 | 30.3 | 16.1 | 53.1 |
| Tₘₐₓ (h) | 0.250 | 0.500 | 0.500 | 0.417 | 0.144 | 34.6 |
| t_{1/2} (h) | 2.25 | 2.68 | 25.5 | 10.1 | 13.3 | 131 |
| Tₗₐₛₜ (h) | 8.00 | 8.00 | 8.00 | 8.00 | — | — |
| AUCₗₐₛₜ | 49.1 | 66.0 | 59.2 | 58.1 | 8.47 | 14.6 |
| (ng · h/mL) | | | | | | |
| AUC_{inf} | 54.1 | 71.3 | 181 | 102 | 68.9 | 67.4 |
| (ng · h/mL) | | | | | | |
| MRTₗₐₛₜ (h) | 3.09 | 2.74 | 3.94 | 3.26 | 0.615 | 18.9 |
| MRT_{inf} (h) | 3.84 | 3.43 | 31.4 | 12.9 | 16.0 | 124 |
| AUC_{Extra} (%) | 9.18 | 7.53 | 67.3 | 28.0 | 34.0 | 122 |
| AUMC_{Extra} | 26.9 | 26.1 | 95.9 | 49.6 | 40.1 | 80.8 |
| (%) | | | | | | |
| AUC Ratio | 0.330 | 0.279 | 0.199 | 0.270 | 0.0663 | 24.6 |
| AUCₗₐₛₜ/Cₘₐₓ | 1.988 | 1.361 | 3.326 | 2.225 | 1.004 | 45.1 |
| (h) | | | | | | |

“—” means not applicable
“ND” means not determined
“D#” means animal number

In group 3: For the SC dose at 1 mg/kg, (6aS)-6-methyl-5,6,6a,7-tetrahydro-4H-dibenzo[de,g]quinoline-10,11-diol showed a plasma Cₘₐₓ of 110±19.4 ng/mL, with Tₘₐₓ at 0.222±0.0479 h. (6aS)-6-methyl-5,6,6a,7-tetrahydro-4H-dibenzo[de,g]quinoline-10,11-diol showed a CSF Cₘₐₓ of 28.5±14.0 ng/mL, with Tₘₐₓ at 0.417±0.144 h. The t_{1/2} of (6aS)-6-methyl-5,6,6a,7-tetrahydro-4H-dibenzo[de,g]quinoline-10,11-diol in plasma and CSF were 5.43±8.36 h and ND, respectively. The AUCₗₐₛₜ and AUC_{inf} values in plasma were 80.1±12.3 ng·h/mL and 88.4±23.8 ng·h/mL, respectively. The AUCₗₐₛₜ and AUC_{inf} values in CSF were 78.2±39.3 ng·h/mL and ND, respectively. The AUCₗₐₛₜ ratio of CSF to total plasma was 0.964±0.456.

The table below demonstrates the individual and mean plasma PK parameters of (6aS)-6-methyl-5,6,6a,7-tetrahydro-4H-dibenzo[de,g]quinoline-10,11-diol in male Beagle dogs following single SC administration of (6aS)-6-methyl-5,6,6a,7-tetrahydro-4H-dibenzo[de,g]quinoline-10,11-diol at 1 mg/kg:

| PK | | | | Mean SC | | CV |
|---|---|---|---|---|---|---|
| Parameters | D03001 | D03002 | D03003 | Plasma | SD | (%) |
| No. points used for t_{1/2} | 3.00 | 5.00 | 4.00 | ND | — | — |
| Cₘₐₓ (ng/mL) | 127 | 88.7 | 113 | 110 | 19.4 | 17.7 |
| Tₘₐₓ (h) | 0.250 | 0.167 | 0.250 | 0.222 | 0.0479 | 21.6 |
| t_{1/2} (h) | 15.1 | 0.564 | 0.641 | 5.43 | 8.36 | 154 |
| Tₗₐₛₜ (h) | 24.0 | 4.00 | 4.00 | ND | — | — |
| AUCₗₐₛₜ | 92.0 | 67.4 | 80.8 | 80.1 | 12.3 | 15.4 |
| (ng · h/mL) | | | | | | |
| AUC_{inf} | 115 | 68.3 | 82.2 | 88.4 | 23.8 | 26.9 |
| (ng · h/mL) | | | | | | |
| MRTₗₐₛₜ (h) | 4.56 | 0.682 | 0.711 | 1.98 | 2.23 | 112 |
| MRT_{inf} (h) | 12.7 | 0.736 | 0.783 | 4.74 | 6.89 | 145 |
| AUC_{Extra} (%) | 19.8 | 1.32 | 1.71 | 7.59 | 10.5 | 139 |
| AUMC_{Extra} | 71.2 | 8.64 | 10.8 | 30.2 | 35.5 | 118 |
| (%) | | | | | | |
| AUCₗₐₛₜ/Cₘₐₓ | 0.724 | 0.760 | 0.715 | 0.733 | 0.024 | 3.2 |
| (h) | | | | | | |

“—” means not applicable
“ND” means not determined
“D#” means animal number

The following table illustrates the individual mean CSF PK parameters of (6aS)-6-methyl-5,6,6a,7-tetrahydro-4H-dibenzo[de,g]quinoline-10,11-diol in male Beagle dogs following single SC administration of (6aS)-6-methyl-5,6,6a,7-tetrahydro-4H-dibenzo[de,g]quinoline-10,11-diol at 1 mg/kg:

| PK | | | | Mean SC | | CV |
|---|---|---|---|---|---|---|
| Parameters | D03001 | D03002 | D03003 | CSF | SD | (%) |
| No. points used for t_{1/2} | 0.00 | 5.00 | 0.00 | ND | — | — |
| Cₘₐₓ (ng/mL) | 17.8 | 23.3 | 44.4 | 28.5 | 14.0 | 49.3 |
| Tₘₐₓ (h) | 0.500 | 0.250 | 0.500 | 0.417 | 0.144 | 34.6 |
| t_{1/2} (h) | ND | 4.79* | ND | ND | ND | ND |
| Tₗₐₛₜ (h) | 8.00 | 8.00 | 8.00 | 8.00 | — | — |
| AUCₗₐₛₜ | 74.1 | 41.0 | 119 | 78.2 | 39.3 | 50.3 |
| (ng · h/mL) | | | | | | |
| AUC_{inf} | ND | 71.2 | ND | ND | ND | ND |
| (ng · h/mL) | | | | | | |
| MRTₗₐₛₜ (h) | 4.27 | 2.81 | 4.07 | 3.72 | 0.792 | 21.3 |
| MRT_{inf} (h) | ND | 7.94 | ND | ND | ND | ND |
| AUC_{Extra} (%) | ND | 42.4 | ND | ND | ND | ND |
| AUMC_{Extra} | ND | 79.6 | ND | ND | ND | ND |
| (%) | | | | | | |
| AUC Ratio | 0.806 | 0.609 | 1.48 | 0.964 | 0.456 | 47.3 |
| AUCₗₐₛₜ/Cₘₐₓ | 4.163 | 1.760 | 2.680 | 2.868 | 1.213 | 42.3 |
| (h) | | | | | | |

“—” means not applicable
“ND” means not determined
“D#” means animal number

Individual and mean plasma concentrations of (6aS)-6-methyl-5,6,6a,7-tetrahydro-4H-dibenzo[de,g]quinoline-10,11-diol in male Beagle dogs following single SL administration of (6aS)-6-methyl-5,6,6a, 7-tetrahydro-4H-dibenzo[de,g]quinoline-10,11-diol at 5 mg/kg (ng/mL):

| Time | | | | Mean SL | | CV |
|---|---|---|---|---|---|---|
| (h) | D01001 | D01002 | D01003 | Plasma | SD | (%) |
| 0 | BQL | BQL | BQL | ND | ND | ND |
| 0.0330 | 2.26 | BQL | BQL | ND | ND | ND |
| 0.167 | 30.9 | 53.6 | 104 | 62.8 | 37.4 | 59.5 |
| 0.250 | 45.2 | 51.2 | 151 | 82.5 | 59.4 | 72.1 |
| 0.500 | 162 | 87.3 | 116 | 122 | 37.7 | 30.9 |
| 1.00 | 75.6 | 116 | 54.5 | 82.0 | 31.3 | 38.1 |
| 2.00 | 11.2 | 21.2 | 10.8 | 14.4 | 5.89 | 40.9 |
| 4.00 | 2.49 | 4.58 | 3.10 | 3.39 | 1.07 | 31.7 |
| 8.00 | 1.51 | BQL | 2.74 | 2.13 | ND | ND |
| 24.0 | 1.49 | BQL | BQL | ND | ND | ND |

Individual and mean CSF concentrations of (6aS)-6-methyl-5,6,6a,7-tetrahydro-4H-dibenzo[de,g]quinoline-10,11-diol in male Beagle dogs following single SL administration of (6aS)-6-methyl-5,6,6a,7-tetrahydro-4H-dibenzo[de,g]quinoline-10,11-diol at 5 mg/kg (ng/mL):

| Time | | | | Mean SL | | CV |
|---|---|---|---|---|---|---|
| (h) | D01001 | D01002 | D01003 | CSF | SD | (%) |
| 0 | BQL | BQL | BQL | ND | ND | ND |
| 0.0330 | BQL | BQL | 1.76 | ND | ND | ND |
| 0.167 | 1.39 | 2.48 | 4.95 | 2.94 | 1.82 | 62.0 |
| 0.250 | 1.77 | 2.61 | 289 | 97.8 | 166 | 169 |
| 0.500 | 11.8 | 6.23 | 99.0 | 39.0 | 52.0 | 133 |
| 1.00 | 17.3 | 13.8 | 54.3 | 28.5 | 22.4 | 78.8 |
| 2.00 | 76.2 | 9.97 | 8.33 | 31.5 | 38.7 | 123 |
| 4.00 | 5.73 | 8.75 | 94.2 | 36.2 | 50.2 | 139 |
| 8.00 | 1.17 | 1.16 | 5.26 | 2.53 | 2.36 | 93.4 |

Individual and mean plasma concentrations of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg (ng/mL):

| Time | | | | Mean PO | | CV |
|---|---|---|---|---|---|---|
| (h) | D02001 | D02002 | D02003 | Plasma | SD | (%) |
| 0 | BQL | BQL | BQL | ND | ND | ND |
| 0.0330 | BQL | BQL | BQL | ND | ND | ND |
| 0.167 | 9.20 | 11.7 | 17.7 | 12.9 | 4.37 | 34.0 |
| 0.250 | 9.58 | 30.7 | 26.3 | 22.2 | 11.1 | 50.2 |
| 0.500 | 84.8 | 72.4 | 78.0 | 78.4 | 6.21 | 7.92 |
| 1.00 | 42.2 | 63.3 | 68.8 | 58.1 | 14.0 | 24.2 |
| 2.00 | 22.9 | 16.5 | 18.1 | 19.2 | 3.33 | 17.4 |
| 4.00 | 15.6 | 14.0 | 8.15 | 12.6 | 3.92 | 31.2 |
| 8.00 | 4.37 | 9.36 | 11.2 | 8.31 | 3.53 | 42.5 |
| 24.0 | BQL | 1.93 | 7.07 | 4.50 | ND | ND |

Individual and mean CSF concentrations of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg (ng/mL)

| Time | | | | Mean PO | | CV |
|---|---|---|---|---|---|---|
| (h) | D02001 | D02002 | D02003 | CSF | SD | (%) |
| 0 | BQL | BQL | BQL | ND | ND | ND |
| 0.0330 | BQL | BQL | BQL | ND | ND | ND |
| 0.167 | 1.33 | 1.93 | 1.13 | 1.46 | 0.416 | 28.5 |
| 0.250 | 24.7 | 18.7 | 7.78 | 17.1 | 8.58 | 50.3 |
| 0.500 | 13.0 | 48.5 | 17.8 | 26.4 | 19.3 | 72.9 |
| 1.00 | 4.85 | 11.8 | 6.46 | 7.70 | 3.64 | 47.2 |
| 2.00 | 8.70 | 4.63 | 2.28 | 5.20 | 3.25 | 62.4 |
| 4.00 | 8.15 | 12.4 | 15.8 | 12.1 | 3.83 | 31.6 |
| 8.00 | 1.53 | 1.39 | 3.32 | 2.08 | 1.08 | 51.7 |

Individual and mean plasma concentrations of (6aS)-6-methyl-5,6,6a,7-tetrahydro-4H-dibenzo[de,g]quinoline-10,11-diol in male Beagle dogs following single SC administration of (6aS)-6-methyl-5,6,6a,7-tetrahydro-4H-dibenzo[de,g]quinoline-10,11-diol at 1 mg/kg (ng/mL)

| Time | | | | Mean SC | | CV |
|---|---|---|---|---|---|---|
| (h) | D03001 | D03002 | D03003 | Plasma | SD | (%) |
| 0 | BQL | BQL | BQL | ND | ND | ND |
| 0.0330 | 8.10 | 4.01 | 25.3 | 12.5 | 11.3 | 90.6 |
| 0.167 | 89.2 | 88.7 | 102 | 93.3 | 7.54 | 8.08 |
| 0.250 | 127 | 84.7 | 113 | 108 | 21.5 | 19.9 |
| 0.500 | 54.1 | 76.6 | 73.4 | 68.0 | 12.2 | 17.9 |
| 1.00 | 11.9 | 22.3 | 27.9 | 20.7 | 8.12 | 39.2 |
| 2.00 | 3.58 | 2.17 | 3.47 | 3.07 | 0.784 | 25.5 |
| 4.00 | 1.93 | 1.11 | 1.52 | 1.52 | 0.410 | 27.0 |
| 8.00 | BQL | BQL | BQL | ND | ND | ND |
| 24.0 | 1.04 | BQL | BQL | ND | ND | ND |

Individual and mean CSF concentrations of (6aS)-6-methyl-5,6,6a,7-tetrahydro-4H-dibenzo[de,g]quinoline-10,11-diol in male Beagle dogs following single SC administration of (6aS)-6-methyl-5,6,6a, 7-tetrahydro-4H-dibenzo[de,g]quinoline-10,11-diol at 1 mg/kg (ng/mL)

| Time | | | | Mean SC | | CV |
|---|---|---|---|---|---|---|
| (h) | D03001 | D03002 | D03003 | CSF | SD | (%) |
| 0 | BQL | BQL | BQL | ND | ND | ND |
| 0.0330 | BQL | 22.6 | 1.73 | 12.2 | ND | ND |
| 0.167 | 8.64 | 13.9 | 7.88 | 10.1 | 3.28 | 32.3 |
| 0.250 | 13.7 | 23.3 | 29.4 | 22.1 | 7.91 | 35.8 |
| 0.500 | 17.8 | 15.6 | 44.4 | 25.9 | 16.0 | 61.8 |
| 1.00 | 4.24 | 9.48 | 21.1 | 11.6 | 8.63 | 74.3 |
| 2.00 | 6.55 | 4.01 | 12.8 | 7.79 | 4.52 | 58.1 |
| 4.00 | 10.2 | 2.39 | 4.11 | 5.57 | 4.10 | 73.7 |
| 8.00 | 10.8 | 4.37 | 37.5 | 17.6 | 17.6 | 100 |

### Example 6: Plasma, CSF, and Brain Pharmacokinetic Study of (6aS)-6-methyl-5,6,6a,7-tetrahydro-4H-dibenzo[de,g]quinoline-10,11-diol Hydrochloride Following Single Oral Administration to Non-Naïve Male Beagle Dogs

The pharmacokinetic parameters of (6aS)-6-methyl-5,6,6a,7-tetrahydro-4H-dibenzo[de,g]quinoline-10,11-diol hydrochloride were evaluated in plasma, cerebral spinal fluid (CSF), and brain following single oral (PO) administration at 10 mg/kg to male beagle dogs.

Animals

Three non-naïve male beagle dogs supplied by Marshall Bioresources (Beijing, China) were used in this study. The animals were confirmed healthy before being assigned to the study. Each animal was given a unique identification number which was marked on ear and written on the cage card. The room(s) were controlled and monitored for relative humidity (targeted mean range 40% to 70%, and any excursion from this range for more than 3 hours will be documented as a deviation) and temperature (targeted mean range 18° C. to 26° C., and any excursion from this range will be documented as a deviation) with 10 to 20 air changes/hour. The rooms were on a 12-hour light/dark cycle except when interruptions are necessitated by study activities.

Animals were fed twice daily. Stock Dogs were fed approximately 220 grams of Certified Dog Diet daily (Beijing Keao Xieli Feed Co., Ltd. Beijing, P. R. China). These amounts could be adjusted as necessary based on food consumption of the group or an individual body weight change. For PO groups, animals were fed at 3:30 to 4:00 pm on the day prior to oral dosing and any remaining food was removed at 7:00 pm. Food was withheld until 4-hour post-dose. Animals were fed once on the day of dosing, with the amount of approximately 220 grams.

Study Design

Three male non-naïve beagle dogs were assigned to this study. Animals were treated with (6aS)-6-methyl-5,6,6a,7-tetrahydro-4H-dibenzo[de,g]quinoline-10,11-diol by single PO administration at 10 mg/kg, which was formulated in 0.5% CMC-Na in water. The detailed dosing and sampling regimens are described in the following two tables:

| | | Dosing | | | | | |
|---|---|---|---|---|---|---|---|
| | No. of | | | Dose | Target Dose | | |
| Group | animals | | Dose | Volume | Concentration | | Dose |
| No. | (male) | Test Article | (mg/kg) | (mL/Kg) | (mg/mL) | Vehicle | Route |
| G1 | 3 | Compound 1 | 10 | 5 | 2 | 0.5% CMC-Na in water | PO(fasted) |

| | Dosage | | Sampling time point (hr) | | | | |
|---|---|---|---|---|---|---|---|
| Group | (mg/kg) | Animal No. | Pre-dose# | 0.033 | 0.167 | 0.25 | 0.5 |
| G1 | 10 | D1001, D1002, | P + CSF | P + CSF | P + CSF | P + CSF | P + CSF + |
| | | D1003 | | | | | Striatum, cortex, |
| | | | | | | | cerebellum |

Dose Administration

PO formulation preparations were processed in study facility on the day of dosing according to instructions provided by the sponsor. The concentrations of compounds in all dosing solutions were determined by HPLC/UV. Two aliquots were taken from each clear and three aliquots were taken from suspension formulations. All formulation samples were stored at 2-8° C. until analyzed by HPLC/UV. All dose formulation samples were analyzed in duplicate using HPLC/UV.

Animals were weighed prior to dose administration on the day of dosing for calculating the actual dose volume. Animals were treated with (6aS)-6-methyl-5,6,6a,7-tetrahydro-4H-dibenzo[de,g]quinoline-10,11-diol hydrochloride by PO administration at 10 mg/kg, which was formulated in 0.5% CMC-Na in water.

Sample Collection and Preparation

In plasma, approximately 0.5 mL blood was collected at each time point. All blood samples were collected from a peripheral vessel. All blood samples were transferred into commercially available tubes containing Potassium (K2) EDTA on wet ice and processed for plasma. Samples were centrifuged (3,000×g for 10 minutes at 2 to 8° C.) within 30 minutes of collection. After centrifugation, the stabilizing agent was added to the harvested plasma within 60 minutes. The samples were kept on ice and protected from sunlight (under yellow light). The stabilizing agent was ascorbic acid 200 mg/mL added in a proportion of 10% in plasma. The plasma samples were divided into two equal samples with each at least 0.2 ml (0.2*2 mL/sample) and transferred into labeled polypropylene micro-centrifuge tubes and stored frozen in a freezer set to maintain 60° C. or lower until analyzed.

In CSF, about 0.1 mL/time point (50 μL*2 aliquots) of CSF were collected via the catheter implanted in the Cisterna Magna of conscious beagle dogs without any sedation. The procedures for CSF collection and handling were as follows: a polypropylene tube was weighed to accuracy of 0.1 mg (weight #1). CSF was collected and weighed the sample tube again (weight #2). Total CSF weight=weight #2−weight #1. The samples were kept on ice and protected from sunlight (under yellow light). Stabilizing agent was added within 60 minutes. The stabilizing agent was ascorbic acid 200 mg/mL added in a proportion of 10% in CSF. CSF samples were frozen on dry ice and stored at −60° C. or lower until bio-analysis.

For brain samples, different regions of brain were collected. Dogs were anesthetized and euthanasia according to SOP and collected the brain. The striatum, cortex and cerebellum were token rapidly and homogenized immediately (within 30 min) after being harvested. All the samples were stored at −60° C. or lower freezer finally.

For tissue homogenization, the homogenization solution was methanol:10 mM PBS=1:2 and set on dry ice. An empty conical tube was weighed, recorded, and set on dry ice. The tissue pieces were picked and placed into the pre-weighed conical tubes. The tube was weighed again and recorded. 5-fold homogenization solution was added to the weighed tissue. The tissues were homogenized by a Polytron (3 strokes or more until homogenous, each 30 seconds) under cold condition (dry ice-ethanol). The sample mixture was very cold but not totally frozen. The stabilizing agent was added within 60 minutes. The stabilizing agent was ascorbic acid 200 mg/mL and added in a proportion of 10% in each homogenized tissue. All the samples were stored at −60° C. or lower freezer finally.

For blank tissue sample homogenization, the tissues were harvested under regular conditions at room temperature. The tissues were weighed and added to a pre-weighed conical tube containing methanol:10 mM PBS=1:2. The tissues were homogenized under room temperature. The stabilizing agent was added. The stabilizing agent was ascorbic acid 200 mg/mL added in a proportion of 10% in each homogenized tissue.

### Preparation of Standards

In plasma, blank male Beagle dog plasma with EDTA-K2 as anti-coagulant was used for the preparation of standards. In CSF, blank artificial CSF was used for the preparation of standards. In cerebellum homogenate, blank male Beagle dog cerebellum homogenate was used for the preparation of calibration standards (C) and QC samples. For striatum homogenate, blank male Beagle dog striatum homogenate was used for the preparation of standards. For cortex homogenate, blank male Beagle dog cortex homogenate was used for the preparation of standards.

About 1.96 mg of (6aS)-6-methyl-5,6,6a,7-tetrahydro-4H-dibenzo[de,g]quinoline-10,11-diol hydrochloride was dissolved in 1.846 mL of DMSO to obtain a 1000 ng/mL stock solution (Stock A).

The preparation procedures of (6aS)-6-methyl-5,6,6a,7-tetrahydro-4H-dibenzo[de,g]quinoline-10,11-diol working solutions are detailed in the table below:

| Source Solution | | | Dilution | Working Solution | | |
|---|---|---|---|---|---|---|
| Source Solution | Conc. | Volume | Volume | Volume | Final Conc. | Working |
| ID | (μg/mL) | (μL) | (μL) | (μL) | (μg/mL) | Solution ID |
| Stock A | 1000 | 30 | 470 | 500 | 60 | WS-Pre |
| WS-Pre | 60 | 40 | 360 | 400 | 6 | WS-C1 |
| WS-C1 | 6 | 120 | 240 | 360 | 2 | WS-C2 |
| WS-C2 | 2 | 160 | 160 | 320 | 1 | WS-C3 |
| WS-C3 | 1 | 80 | 320 | 400 | 0.2 | WS-C4 |
| WS-C4 | 0.2 | 160 | 160 | 320 | 0.1 | WS-C5 |
| WS-C5 | 0.1 | 80 | 320 | 400 | 0.02 | WS-C6 |
| WS-C6 | 0.02 | 80 | 320 | 400 | 0.004 | WS-C7 |
| WS-C7 | 0.004 | 160 | 160 | 320 | 0.002 | WS-C8 |
| Stock A | 1000 | 30 | 595 | 625 | 48 | WS-QC-Pre |
| WS-QC-Pre | 48 | 40 | 360 | 400 | 4.8 | WS-QC1 |
| WS-QC1 | 4.8 | 120 | 240 | 360 | 1.6 | WS-QC2 |
| WS-QC2 | 1.6 | 20 | 380 | 400 | 0.08 | WS-QC3 |
| WS-QC3 | 0.08 | 30 | 370 | 400 | 0.006 | WS-QC4 |
| Stock A | 1000 | 30 | 270 | 300 | 100 | WS-DQC1-Pre |
| WS-DQC1-Pre | 100 | 40 | 360 | 400 | 10 | WS-DQC2 |

Dilution Solvent: 0.02% 2-mercaptoethanol in MeOH for (6aS)-6-methyl-5,6,6a,7-tetrahydro-4H-dibenzo[de,g]quinoline-10,11-diol hydrochloride

Preparation details of the (6aS)-6-methyl-5,6,6a,7-tetrahydro-4H-dibenzo[de,g]quinoline-10,11-diol calibration and QC standards in plasma, CSF, and tissue homogenate are shown in the following table:

| Working Solution | | | Matrix | Calibration Standard | | |
|---|---|---|---|---|---|---|
| Source | Conc. | Volume | Volume | Volume | Final Conc. | Calibration |
| Solution ID | (μg/mL) | (μL) | (μL) | (μL) | (ng/mL) | Standard ID |
| WS-C1 | 6 | 2 | 38 | 40 | 300 | C1 |
| WS-C2 | 2 | 2 | 38 | 40 | 100 | C2 |
| WS-C3 | 1 | 2 | 38 | 40 | 50 | C3 |
| WS-C4 | 0.2 | 2 | 38 | 40 | 10 | C4 |
| WS-C5 | 0.1 | 2 | 38 | 40 | 5 | C5 |
| WS-C6 | 0.02 | 2 | 38 | 40 | 1 | C6 |
| WS-C7 | 0.004 | 2 | 38 | 40 | 0.2 | C7 |
| WS-C8 | 0.002 | 2 | 38 | 40 | 0.1 | C8 |

| Source | Conc. | Volume | Volume | Volume | Final Conc. | |
|---|---|---|---|---|---|---|
| Solution ID | (μg/mL) | (μL) | (μL) | (μL) | (ng/mL) | QC ID |
| WS-QC1 | 4.8 | 2 | 38 | 40 | 240 | QC1 |
| WS-QC2 | 1.6 | 2 | 38 | 40 | 80 | QC2 |
| WS-QC3 | 0.08 | 2 | 38 | 40 | 4 | QC3 |
| WS-QC4 | 0.006 | 2 | 38 | 40 | 0.3 | QC4 |
| WS-DQC2 | 10 | 2 | 38 | 40 | 500 | DQC |

The concentrations of calibration and QC samples are shown in the table below:

| | | Calibration | |
|---|---|---|---|
| | | Standard Samples | Quality Control Samples |
| Compound | Matrix | Conc. (ng/mL) | Conc. (ng/mL) |
| (6aS)-6-methyl-5,6,6a,7- | Male Beagle dog | 0.1, 0.2, 1, 5, 10, 50, | |
| tetrahydro-4H- | Cortex homogenate | 100, 300 | |
| dibenzo [de,g] quinoline- | | | |
| 10,11-diol HCl | | | |
| (6aS)-6-methyl-5,6,6a,7- | Male Beagle dog | 1, 2, 10, 50, 100, 500, | |
| tetrahydro-4H- | plasma | 1000, 3000 | |
| dibenzo [de, g] quinoline- | | | |
| 10,11-diol HCl | | | |
| (6aS)-6-methyl-5,6,6a,7- | Male Beagle dog | 0.1, 0.2, 1, 5, 10, 50, | |
| tetrahydro-4H- | Striamu homogenate | 100, 300 | |
| dibenzo [de, g] quinoline- | | | |
| 10,11-diol HCl | | | |
| (6aS)-6-methyl-5,6,6a,7- | Male Beagle dog | 0.1, 0.2, 1, 5, 10, 50, | 0.3, 4, 80, 240, 500 (DQC) |
| tetrahydro-4H- | Cerebellum | 100, 300 | 0.3, 4, 80, 240 |
| dibenzo [de, g] quinoline- | homogenate | | |
| 10,11-diol HCl | | | |
| (6aS)-6-methyl-5,6,6a,7- | Male Beagle dog | 0.1, 0.2, 1, 5, 10, 50, | 0.3, 4, 80, 240 |
| tetrahydro-4H- | plasma | 100, 300 | |
| dibenzo [de, g] quinoline- | | | |
| 10,11-diol HCl | | | |

Sample Treatment

The tissue (Cerebellum, Striatum and Cortex) was homogenized using homogenizing buffer (methanol:10 mM PB S=1:2) at the ratio of 1:5 (1 g tissue with 5 mL buffer). An aliquot of 20 μL sample with 400 μL internal standard (100 ng/mL Labetalol & 100 ng/mL Dexamethasone & 100 ng/mL Tolbutamide & 100 ng/mL Verapamil & 100 ng/mL Glyburide & 200 ng/mL Diclofenac in ACN), the mixture was vortex-mixed well and centrifuged at 13000 rpm for 10 min, 4° C. 70 μL supernatant was then mixed with 70 μL water, 3 μL sample was injected for LC-MS/MS analysis.

For the dilutions: a dilution factor as 5—an aliquot of 4 μL sample was added with 16 μL blank plasma; and a dilution factor as 10—An aliquot of 2 μL sample was added with 18 μL blank plasma.

LC-MS/MS Conditions

The LC-MS/MS system consisted of an ACQUITY UPLC System and API 4000 mass spectrometer. Chromatographic separation was performed on a ACQUITY UPLC HSS T3 (2.1×50 mm, 1.8 μm) column. The mobile phase was composed of A: 0.1% formic acid and 2 mM HCOONH₄ in Water:ACN (v:v, 95:5); and B: 0.1% formic acid & 2 mM HCOONH₄ in ACN:Water (v:v, 95:5). The flow rate was 0.6 mL/min. The injection volume was 3 μL. The following gradient was implemented:

| | Flow Rate | | |
|---|---|---|---|
| Time (min) | (mL/min) | A (%) | B (%) |
| Initial | 0.600 | 98.0 | 2.0 |
| 0.20 | 0.600 | 98.0 | 2.0 |
| 0.90 | 0.600 | 55.0 | 45.0 |
| 1.10 | 0.600 | 5.0 | 95.0 |
| 1.60 | 0.600 | 5.0 | 95.0 |
| 1.61 | 0.600 | 98.0 | 2.0 |
| 1.80 | 0.600 | 98.0 | 2.0 |

Positive mode electrospray ionization (ESI) was performed to obtain a protonated ion of (6aS)-6-methyl-5,6,6a,7-tetrahydro-4H-dibenzo[de,g]quinoline-10,11-diol, and Verampil (IS). A multiple reaction monitoring (MRM) method was selected for quantitative analysis. The (6aS)-6-methyl-5,6,6a,7-tetrahydro-4H-dibenzo[de,g]quinoline-10,11-diol and Verampil dependent parameters are listed as follows:

| | Ion | Collision Energy | Retention Time |
|---|---|---|---|
| Compound Name | Transition | (eV) | (min) |
| (6aS)-6-methyl- | 268.0/219.2 | 26 | 0.96 |
| 5,6,6a,7-tetrahydro- | | | |
| 4H- | | | |
| dibenzo[de,g] | | | |
| quinoline-10,11-diol | | | |
| Verapamil | 455.2/164.9 | 22 | 1.3 |

Data acquisition was performed by Analyst 1.6.3 software was used for processing the data of all samples.

Results

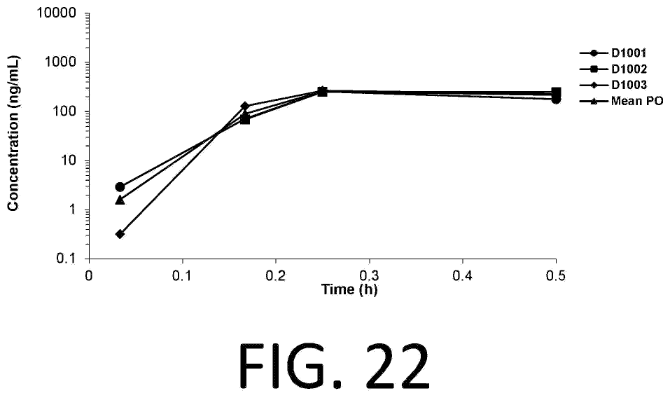

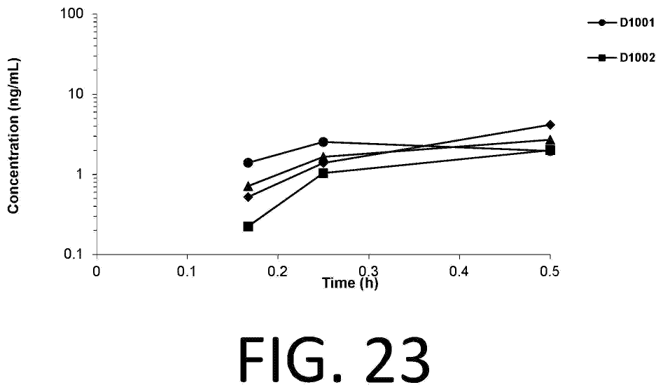

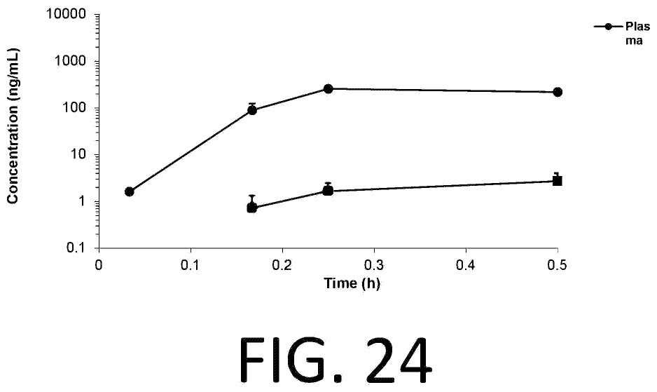

The individual and mean plasma concentration-time profiles are illustrated from FIGS. 22-24.

For the PO dose at 10 mg/kg, (6aS)-6-methyl-5,6,6a,7-tetrahydro-4H-dibenzo[de,g]quinoline-10,11-diol showed a plasma Cₘₐₓ of 257±7.21 ng/mL, with Tₘₐₓ at 0.250±0.000 h. (6aS)-6-methyl-5,6,6a,7-tetrahydro-4H-dibenzo[de,g]quinoline-10,11-diol showed a CSF Cₘₐₓ of 2.90±1.12 ng/mL, with Tₘₐₓ at 0.417±0.144 h. The AUCₗₐₛₜ values in plasma and CSF were 80.0±7.29 ng·h/mL and 0.703±0.218 ng·h/mL, respectively. The AUCₗₐₛₜ ratio of CSF to plasma was 0.00888±0.00310.

The table below demonstrates the individual and mean plasma PK parameters of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg:

| PK Parameters | D1001 | D1002 | D1003 | Mean PO | SD | CV (%) |
|---|---|---|---|---|---|---|
| No. points used for t_{1/2} | 0.00 | 0.00 | 0.00 | 0.00 | — | — |
| Cₘₐₓ (ng/mL) | 255 | 251 | 265 | 257 | 7.21 | 2.81 |
| Tₘₐₓ (h) | 0.250 | 0.250 | 0.250 | 0.250 | 0.00 | 0.00 |
| t_{1/2} (h) | ND | ND | ND | ND | ND | ND |
| Tₗₐₛₜ (h) | 0.500 | 0.500 | 0.500 | 0.500 | — | — |
| AUCₗₐₛₜ (ng · h/mL) | 72.1 | 81.6 | 86.4 | 80.0 | 7.29 | 9.11 |
| AUC_{inf} (ng · h/mL) | ND | ND | ND | ND | ND | ND |
| MRTₗₐₛₜ (h) | 0.328 | 0.337 | 0.323 | 0.329 | 0.00717 | 2.18 |
| MRT_{inf} (h) | ND | ND | ND | ND | ND | ND |
| AUC_{Extra} (%) | ND | ND | ND | ND | ND | ND |
| AUMC_{Extra} (%) | ND | ND | ND | ND | ND | ND |
| AUCₗₐₛₜ/Cₘₐₓ (h) | 0.283 | 0.325 | 0.326 | 0.311 | 0.025 | 7.9 |

“—” means not applicable
“ND” means not determined
“D#” means animal number

The following table illustrates the Individual and mean CSF PK parameters of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg:

| PK Parameters | D1001 | D1002 | D1003 | Mean PO | SD | CV (%) |
|---|---|---|---|---|---|---|
| No. points used for t_{1/2} | 0.00 | 0.00 | 0.00 | 0.00 | — | — |
| Cₘₐₓ (ng/mL) | 2.54 | 2.00 | 4.16 | 2.90 | 1.12 | 38.8 |
| Tₘₐₓ (h) | 0.250 | 0.500 | 0.500 | 0.417 | 0.144 | 34.6 |
| t_{1/2} (h) | ND | ND | ND | ND | ND | ND |
| Tₗₐₛₜ (h) | 0.500 | 0.500 | 0.500 | 0.500 | — | — |
| AUCₗₐₛₜ (ng · h/mL) | 0.840 | 0.451 | 0.817 | 0.703 | 0.218 | 31.0 |
| AUC_{inf} (ng · h/mL) | ND | ND | ND | ND | ND | ND |
| MRTₗₐₛₜ (h) | 0.312 | 0.383 | 0.403 | 0.366 | 0.0475 | 13.0 |
| MRT_{inf} (h) | ND | ND | ND | ND | ND | ND |
| AUC_{Extra} (%) | ND | ND | ND | ND | ND | ND |
| AUMC_{Extra} (%) | ND | ND | ND | ND | ND | ND |
| CSF/Plasma AUC Ratio | 0.0117 | 0.00553 | 0.00945 | 0.00888 | 0.00310 | 35.0 |
| AUCₗₐₛₜ/Cₘₐₓ (h) | 0.331 | 0.226 | 0.196 | 0.251 | 0.071 | 28.2 |

“—” means not applicable
“ND” means not determined
“D#” means animal number

The concentration of (6aS)-6-methyl-5,6,6a,7-tetrahydro-4H-dibenzo[de,g]quinoline-10,11-diol in plasma, striatum, cortex and cerebellum at 0.5 hour post-dose were 218±36.5 ng/mL, 145±22.7 ng/g, 151±36.2 ng/g and 108±19.1 ng/g, respectively. The ratios of striatum/plasma, cortex/plasma and cerebellum/plasma were 0.666±0.0544, 0.704±0.184 and 0.505±0.117, respectively.

The table below demonstrates the Individual and mean plasma concentrations of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg (ng/mL):

| Time | | | | Mean | | CV |
|---|---|---|---|---|---|---|
| (h) | D1001 | D1002 | D1003 | PO | SD | (%) |
| 0.0000 | BQL | BQL | BQL | ND | ND | ND |
| 0.0330 | 2.92 | BQL | 0.319 | 1.62 | ND | ND |
| 0.167 | 71.0 | 68.8 | 129 | 89.5 | 34.0 | 38.0 |
| 0.250 | 255 | 251 | 265 | 257 | 7.32 | 2.85 |
| 0.500 | 178 | 250 | 227 | 218 | 36.5 | 16.7 |

“BQL” means below the quantifiable limit (1.00 ng/mL)
“ND” means not determined as more than half (>50%) of the individual values are not quantifiable
“D#” means animal number

The following table illustrates the individual and mean CSF concentrations of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg (ng/mL):

| Time | | | | Mean | | CV |
|---|---|---|---|---|---|---|
| (h) | D1001 | D1002 | D1003 | PO | SD | (%) |
| 0.0000 | BQL | BQL | BQL | ND | ND | ND |
| 0.0330 | BQL | BQL | BQL | ND | ND | ND |
| 0.167 | 1.40 | 0.224 | 0.523 | 0.715 | 0.609 | 85.3 |
| 0.250 | 2.54 | 1.04 | 1.39 | 1.66 | 0.786 | 47.5 |
| 0.500 | 1.96 | 2.00 | 4.16 | 2.71 | 1.26 | 46.5 |

“BQL” means below the quantifiable limit (1.00 ng/mL)
“ND” means not determined as more than half (>50%) of the individual values are not quantifiable
“D#” means animal number

The table below demonstrates the individual and mean striatum, cortex and cerebellum concentrations of (6aS)-6-methyl-5,6,6a,7-tetrahydro-4H-dibenzo[de,g]quinoline-10,11-diol in male Beagle dogs following single PO administration of (6aS)-6-methyl-5,6,6a,7-tetrahydro-4H-dibenzo[de,g]quinoline-10,11-diol at 10 mg/kg (ng/g):

| | Time | | | | Mean | | CV |
|---|---|---|---|---|---|---|---|
| Matrix | (h) | D1001 | D1002 | D1003 | PO | SD | (%) |
| Striatum | 0.500 | 127 | 170 | 137 | 145 | 22.7 | 15.7 |
| Cortex | 0.500 | 137 | 124 | 192 | 151 | 36.2 | 24.0 |
| Cerebellum | 0.500 | 102 | 92.4 | 129 | 108 | 19.1 | 17.7 |

### Example 7: Plasma Pharmacokinetic Study of (6aS)-6-methyl-5,6,6a,7-tetrahydro-4H-dibenzo[de,g]quinoline-10,11-diol Following Subcutaneous and Oral Administration in Fasted Male Cynomolgus Monkeys

The pharmacokinetic profile of (6aS)-6-methyl-5,6,6a,7-tetrahydro-4H-dibenzo[de,g]quinoline-10,11-diol was evaluated in male Cynomolgus Monkeys. 3 male Cynomolgus Monkeys were administrated a single Subcutaneous dose of 1.14 mg/kg and a solution formulation at a matching dose level. The (6aS)-6-methyl-5,6,6a,7-tetrahydro-4H-dibenzo[de,g]quinoline-10,11-diol pharmacokinetics profile was evaluated in male Cynomolgus Monkeys. Three male Cynomolgus Monkeys were administrated a single oral dose of 11.4 mg/kg and a solution formulation at a matching dose level. The vehicle for SC formulation was 0.1% sodium metabisulfite solution and PO formulation was 0.5% CMC-Na in water.

Animals

Male Cynomolgus Monkeys were purchased from Topgene Biotechnology. The animals were 2-5 years old with body weights of 2.0-5.0 kg on the dosing date. The animals were housed in a 12-hour light/12-hour dark cycle environment and had free access to food and water.

All animals for SC administration had free access to food and water. All animals for PO administration were fasted overnight prior to dosing and will be fed approximately 2 hours after dosing.

Study Design

Six male Cynomolgus Monkeys were assigned to this study consisting of two groups. In group 1, animals were treated with (6aS)-6-methyl-5,6,6a,7-tetrahydro-4H-dibenzo[de,g]quinoline-10,11-diol by single SC administration at 1.14 mg/kg, which was formulated in 0.1% sodium metabisulfite. In group 2, animals were treated with (6aS)-6-methyl-5,6,6a,7-tetrahydro-4H-dibenzo[de,g]quinoline-10,11-diol by PO administration at 11.4 mg/kg, which was formulated in 0.5% CMC-Na in water. The dosing regimens and formulation (vehicle) compositions are described in the following table below:

| | | Dose Level | Dose Volume | Conc. | Administration | No. of |
|---|---|---|---|---|---|---|
| Group | Treatment | (mg/kg) | (ml/kg) | (mg/ml) | Route | Animals |
| 1 | (6aS)-6-methyl- | 1.14 | 1 | 1.14 | SC | 3 |
| 2 | 5,6,6a,7- | 11.4 | 5 | 2.28 | PO | 3 |
| | tetrahydro-4H- | | | | | |
| | dibenzo[de,g]quin- | | | | | |
| | oline-10,11-diol | | | | | |

Dose Administration

Animals were weighed prior to dose administration on the day of dosing for calculating the actual dose volume. The dosages were prepared as illustrated in the table below:

| | | Free | | | Weight | Conc | Volume | 0.1% sodium |
|---|---|---|---|---|---|---|---|---|
| Compound | Route | MW | MW | FW | (mg) | (mg/mL) | (mL) | metabisulfite |
| (6aS)-6-methyl- | SC | 267.32 | 303.79 | 312.79 | 15.35 | 1.14 | 13.077 | 13.077 |
| 5,6,6a,7- | | | | | | | | |
| tetrahydro-4H- | | | | | | | | |
| dibenzo[de,g]quin- | | | | | | | | |
| oline-10,11-diol | | | | | | | | |
| | | Free | | | Weight | Conc | Volume | 0.5% CMC- |
| Compound | Route | MW | MW | FW | (mg) | (mg/mL) | (mL) | Na |
| (6aS)-6-methyl- | PO | 267.32 | 303.79 | 312.79 | 119.00 | 2.28 | 50.691 | 50.691 |
| 5,6,6a,7- | | | | | | | | |
| tetrahydro-4H- | | | | | | | | |
| dibenzo[de,g]quin- | | | | | | | | |
| oline-10,11-diol | | | | | | | | |

Sample Collection

Blood samples were taken at the following times:

| Group | PK time points |
|---|---|
| SC | Pre-dose, 2 min, 5 min, 10 min, 15 min, 30 min, |
| | 1, 2, 4, 8 and 24 hr post dose |
| PO | Pre-dose, 5 min, 10 min, 15 min, 30 min, |
| | 1, 2, 4, 8 and 24 hr post dose |

Whole blood samples were gently inverted several times to ensure anticoagulation then placed on wet ice prior to centrifugation. Within 30 minutes of collection, the samples were centrifuged at approximately 4,000×g (force) for 5 minutes at 2 to 8° C. to obtain plasma. The stabilizing agent, ascorbic acid at 200 mg/mL, was added in a proportion of 10% in plasma. The plasma samples were stored in a freezer at −75±15° C. prior to analysis. The completion time from collection was less than 60 min. The resultant plasma samples will be immediately separated divided into 2 aliquots (˜75 μL each) and transferred to cryogenic vial. Samples will be maintained on wet ice throughout processing, and stored in a freezer at −75±15° C. prior to analysis. Samples should be protected from ambient light (under sodium lamp).

### Preparation of Standards

Standards were prepared with 0.96 mg of (6aS)-6-methyl-5,6,6a,7-tetrahydro-4H-dibenzo[de,g]quinoline-10,11-diol added to 0.820 mL DMSO to obtain a 1 mg/mL standard stock solution (free form).

Calibration standard working solutions were prepared at concentrations of 2, 5, 10, 20, 100, 500, 1000, 5000, 10000 ng/mL by serial dilution of the standard stock solution in 1:1 acetonitrile:water. Quality control working solutions at concentrations of 5, 10, 20, 500 and 8000 ng/mL were prepared by serial dilution of the standard stock solution in 1:1 acetonitrile:water. The QC samples were prepared on the day of analysis in the same way as calibration standards.

Sample Treatment

Samples were prepared with 5 μL of each calibration standard working solution (2, 5, 10, 20, 100, 500, 1000, 5000, 10000 ng/mL) added to 50 μL of the blank Cynomolgus Monkeys plasma to achieve calibration standards concentrations of 0.2, 0.5, 1, 2, 10, 50, 100, 500, 1000 ng/mL (relative to a 50-4, plasma aliquot). Quality Control (QC) samples at 0.5 ng/mL (low-1), 1 ng/mL (low-2), 2 ng/mL (low-3), 50 ng/mL (mid) and 800 ng/mL (high) for plasma were prepared independently for those used for the calibration curves.

Total volumes of 55 μL of standards, 55 μL of QC samples and 55 μL of unknown samples (50 μL of plasma with 5 μL of 1:1 acetonitrile:water) were added to 200 μL of acetonitrile containing IS to precipitate proteins. Then the samples were vortexed for 30 sec. After centrifugation at 4° C., 3900 rpm for 15 min, supernatant was diluted 5 times with water, and 2 μL of the diluted supernatant was injected into the LC-MS/MS system for quantitative analysis. All of the samples were processed on ice.

LC-MS/MS Conditions

The LC-MS/MS system consisted of two Shimadzu LC-30AD pumps, a DGU-20A5R degasser, a Rack changer II and an AB Sciex Triple Quad 5500 LC/MS/MS mass spectrometer.

Chromatographic separation was performed on Agilent ZORBAX XDB-Phenyl 5 μm (50×2.1 mm) at room temperature. The mobile phase was composed of A: 5% Acetonitrile/95% water (0.1% Formic acid); B: 95% Acetonitrile/5% water (0.1% Formic acid). The flow rate was 0.6 mL/min. The injection volume was 2 μL.

Positive mode electrospray ionization (ESI) was performed on a Turbo VR® ion source to obtain a protonated ion of (6aS)-6-methyl-5,6,6a,7-tetrahydro-4H-dibenzo[de,g]quinoline-10,11-diol and Dexamethasone (IS). A multiple reaction monitoring (MRM) method was selected for quantitative analysis. The optimized transitions were 268.05/237.10 Da and 393.14/373.10 Da for (6aS)-6-methyl-5,6,6a,7-tetrahydro-4H-dibenzo[de,g]quinoline-10,11-diol and Dexamethasone, respectively. The instrument parameters were set as follows: ion spray voltage: 5500 V; curtain gas: 40 psi; ion source gas 1: 50 psi; ion source gas 2: 50 psi; temperature: 450° C. The compound dependent parameters are listed as follows:

| | (6aS)-6-methyl-5,6,6a,7- | |
|---|---|---|
| | tetrahydro-4H- | |
| | dibenzo[de,g]quinoline- | Dexamethasone |
| | 10,11-diol | (IS) |
| Precursor/ | 268.05/237.10 | 393.14/373.10 |
| product | | |
| DP | 131.00 | 146.00 |
| CE | 25.00 | 13.00 |
| CXP | 18.00 | 16.00 |

HPLC flow rate was set at 0.6 mL/min with the following conditions:

| Time (min) | A (%) | B (%) |
|---|---|---|
| 0.01 | 95.0 | 5.00 |
| 0.20 | 95.0 | 5.00 |
| 1.90 | 5.00 | 95.0 |
| 2.20 | 5.00 | 95.0 |
| 2.21 | 95.0 | 5.00 |
| 2.50 | 95.0 | 5.00 |

Results

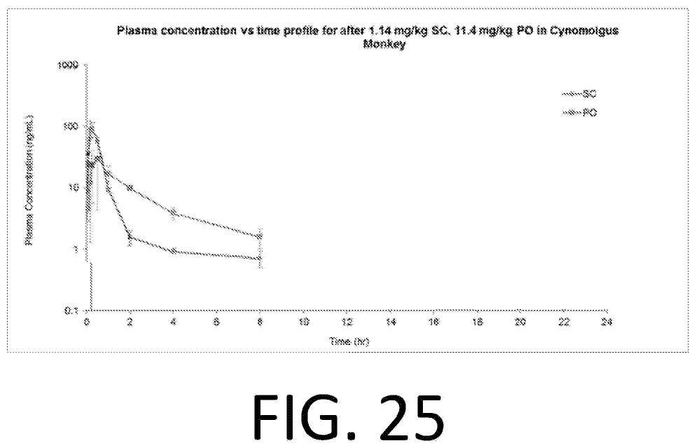

The mean plasma concentration-time profiles after PO and SC administration are illustrated from FIG. 25.

Following the SC (1.14 mg/kg) administration the t_{1/2} was 5.20 hr, Tₘₐₓ was 0.195±0.048 hr, Cₘₐₓ was 92±28 ng/mL, MRT_{Inf} was 1.96 hr, AUC₀₋₂₄ hr was 60.5±11.8 h*ng/mL, and AUC_{inf} was 58.2 h*ng/mL in Cynomolgus Monkeys.

The table below demonstrates shows the plasma pharmacokinetic parameters of (6aS)-6-methyl-5,6,6a,7-tetrahydro-4H-dibenzo[de,g]quinoline-10,11-diol following SC administration to male Cynomolgus Monkeys at 1.14 mg/kg:

| PK parameters | Unit | 170353C | 170291C | 165161C | Mean | SD | CV(%) |
|---|---|---|---|---|---|---|---|
| t_{1/2} | h | 4.68 | 5.72 | NA | 5.20 | NA | NA |
| Tₘₐₓ | h | 0.250 | 0.167 | 0.167 | 0.195 | 0.048 | 24.6 |
| Cₘₐₓ | ng/mL | 74.1 | 77.4 | 124 | 92 | 28 | 30.4 |
| AUCₗₐₛₜ | h*ng/mL | 52.1 | 55.5 | 74.0 | 60.5 | 11.8 | 19.5 |
| AUC_{Inf} | h*ng/mL | 56.0 | 60.3 | NA | 58.2 | NA | NA |
| AUC_% Extrap_obs | % | 7.05 | 7.98 | NA | 7.52 | NA | NA |
| MRT_{Inf} | h | 1.89 | 2.04 | NA | 1.96 | NA | NA |
| AUCₗₐₛₜ/D | h*mg/mL | 45.7 | 48.7 | 64.9 | 53.1 | 10.4 | 19.5 |
| F | % | NA | NA | NA | NA | NA | NA |
| AUCₗₐₛₜ/Cₘₐₓ | h | 0.703 | 0.717 | 0.597 | 0.672 | 0.066 | 9.8 |

Following the PO (11.4 mg/kg) t_{1/2} was 2.26±0.56 hr, Tₘₐₓ was 0.667±0.289 hr, Cₘₐₓ was 34.7±17.9 ng/mL, MRT_{Inf} was 2.92±0.92 hr, AUC₀₋₂₄ hr was 58.1±12.4 h*ng/mL, and AUC_{inf} was 63.5±11.9 h*ng/mL in Cynomolgus Monkeys.

The following table illustrates the plasma pharmacokinetic parameters of (6aS)-6-methyl-5,6,6a,7-tetrahydro-4H-dibenzo[de,g]quinoline-10,11-diol following PO administration to male Cynomolgus Monkeys at 11.4 mg/kg:

| PK parameters | Unit | 170295C | 174223C | 174753C | Mean | SD | CV(%) |
|---|---|---|---|---|---|---|---|
| t_{1/2} | h | 1.80 | 2.88 | 2.09 | 2.26 | 0.56 | 24.8 |
| Tₘₐₓ | h | 0.500 | 1.00 | 0.500 | 0.67 | 0.29 | 43.3 |
| Cₘₐₓ | ng/mL | 27.7 | 21.4 | 55.0 | 34.7 | 17.9 | 51.5 |
| AUCₗₐₛₜ | h*ng/mL | 54.2 | 48.2 | 72.0 | 58.1 | 12.4 | 21.3 |
| AUC_{Inf} | h*ng/mL | 56.6 | 56.6 | 77.3 | 63.5 | 11.9 | 18.8 |
| AUC_% Extrap_obs | % | 4.31 | 14.8 | 6.80 | 8.6 | 5.5 | 63.5 |
| MRT_{Inf} | h | 2.46 | 3.98 | 2.33 | 2.92 | 0.92 | 31.3 |
| AUCₗₐₛₜ/D | h*mg/mL | 4.75 | 4.23 | 6.32 | 5.10 | 1.09 | 21.3 |
| F | % | NA | NA | NA | NA | NA | NA |
| AUCₗₐₛₜ/Cₘₐₓ | h | 1.957 | 2.252 | 1.309 | 1.839 | 0.482 | 26.2 |

(6aS)-6-methyl-5,6,6a,7-tetrahydro-4H-dibenzo[de,g]quinoline-10,11-diol Plasma Concentrations Following SC Administration to Male Cynomolgus Monkeys at 1.14 mg/kg:

| Time | Concentration (ng/mL) | | | Mean | SD | CV |
|---|---|---|---|---|---|---|
| (h) | 170353C | 170291C | 165161C | (ng/mL) | (ng/mL) | (%) |
| 0 | BLOQ | BLOQ | BLOQ | NA | NA | NA |
| 0.033 | 1.88 | 12.5 | 12.6 | 9.0 | 6.2 | 68.6 |
| 0.083 | 5.47 | 40.3 | 65.3 | 37.1 | 30.1 | 81.2 |
| 0.167 | 66.5 | 77.4 | 124 | 89 | 30 | 34.1 |
| 0.25 | 74.1 | 76.3 | 119 | 90 | 26 | 28.4 |
| 0.5 | 52.4 | 57.0 | 65.1 | 58.2 | 6.4 | 11.1 |
| 1 | 10.3 | 8.14 | 9.44 | 9.28 | 1.07 | 11.5 |
| 2 | 1.44 | 1.23 | 2.02 | 1.56 | 0.41 | 26.3 |
| 4 | 1.01 | 0.884 | 0.844 | 0.912 | 0.085 | 9.36 |
| 8 | 0.585 | 0.584 | 0.959 | 0.710 | 0.216 | 30.5 |
| 24 | BLOQ | BLOQ | BLOQ | NA | NA | NA |

(6aS)-6-methyl-5,6,6a,7-tetrahydro-4H-dibenzo[de,g]quinoline-10,11-diol Plasma Concentrations Following PO Administration to Male Cynomolgus Monkeys at 11.4 mg/kg

| Time | Concentration (ng/mL) | | | Mean | SD | CV |
|---|---|---|---|---|---|---|
| (h) | 170295C | 174223C | 174753C | (ng/mL) | (ng/mL) | (%) |
| 0 | BLOQ | BLOQ | BLOQ | NA | NA | NA |
| 0.083 | 2.55 | 0.786 | 10.1 | 4.5 | 5.0 | 111 |
| 0.167 | 12.3 | 1.26 | 23.1 | 12.2 | 10.9 | 89.4 |
| 0.25 | 27.2 | 3.90 | 38.1 | 23.0 | 17.5 | 75.7 |
| 0.5 | 27.7 | 5.15 | 55.0 | 29.3 | 25.0 | 85.2 |
| 1 | 10.8 | 21.4 | 18.2 | 16.8 | 5.4 | 32.1 |
| 2 | 9.42 | 9.30 | 10.8 | 9.8 | 0.8 | 8.33 |
| 4 | 4.63 | 3.81 | 2.98 | 3.80 | 0.82 | 21.7 |
| 8 | 0.941 | 2.02 | 1.74 | 1.57 | 0.56 | 35.8 |
| 24 | BLOQ | BLOQ | BLOQ | NA | NA | NA |

### Example 8: Pharmacokinetic and Brain Penetration Study Following Single and Repeated Dose of (6aS)-6-methyl-5,6,6a,7-tetrahydro-4H-dibenzo[de,g]quinoline-10,11-diol Hydrochloride Via PO Administration in Male C57BL/6J Mouse

(6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol pharmacokinetic profiles were evaluated in male C57BL/6J mice (N=3). 9 male C57BL/6J mice following administration of a single and repeated PO dose of 4.40 mg/kg. The vehicle for the subcutaneous formulation contained “0.1% sodium metabisulfite solution”. During this PK study, there were no obvious drug-related changes in clinical signs and behavioral responses. There was no animal death related to drug administration.

Animals

Male C57BL/6J mice were purchased from Si Bei Fu Laboratory Animal Technology Co. Ltd (Beijing, China). The animals were about 8 weeks old with body weights of 20-30 g on the dosing date. The animals were housed in a 12-hour light/12-hour dark cycle environment and had free access to food and water. All animals were fasted overnight and fed after 4 hours collection.

Study Design

(6aS)-6-methyl-5,6,6a,7-tetrahydro-4H-dibenzo[de,g]quinoline-10,11-diol hydrochloride hemihydrate was administered to both groups 1 and 2 via oral administration (25 mg/kg) at 10 mL/kg. Blood, CSF, and brain samples were collected from group 1 at 5 min., 15 min., 30 min., 1 hr., 2 hrs., 3 hrs., and 4 hrs. following administration of a single dose. Blood, CSF, and brain samples were collected from group 2 at 0 min., 5 min., 15 min., 30 min., 1 hr., 2 hrs., 3 hrs., 4 hrs., 8 hrs., 12 hrs., 24 hrs., and 48 hrs. post last day (Day 4).

| | | Dose Level | Dose | | | |
|---|---|---|---|---|---|---|
| | | (mg/kg) (salt | Volume | Conc. | | |
| | | hhemiydrate | (mL/ | (mg/ | | No. of |
| Group | Treatment | form) | kg) | mL) | Route | Animals |
| 1 | (6aS)-6-methyl-5,6,6a,7- | 25 | 10 | 2.5 | PO (Single | 3/male |
| | tetrahydro-4H- | | | | Dose) | |
| | dibenzo[de,g]quinoline- | | | | | |
| | 10,11-diol hydrochloride | | | | | |
| 2 | (6aS)-6-methyl-5,6,6a,7- | 25 | 10 | 2.5 | PO (Once | 6/male |
| | tetrahydro-4H- | | | | daily for 4 | |
| | dibenzo[de,g]quinoline- | | | | days) | |
| | 10,11-diol hydrochloride | | | | | |

Dosage and Administration

The formulation was freshly made prior to use. Preparation of formulation for oral dosing are as follows:

Preparation of stock solution: 1.55 mg of (6aS)-6-methyl-5,6,6a,7-tetrahydro-4H-dibenzo[de,g]quinoline-10,11-diol hydrochloride hemihydrate was dissolved in 1.505 mL DMSO with vortex and sonification to prepare 1 mg/mL stock solution in DMSO.

Preparation of Day 1 (25 mg/kg, 10 mL/kg) formulation: 2.5 mg/mL of “10% HP-(3-CD in water” solution was prepared by dissolving 61.83 mg of (6aS)-6-methyl-5,6,6a,7-tetrahydro-4H-dibenzo[de,g]quinoline-10,11-diol hydrochloride hemihydrate in 24.020 mL of 10% HP-(3-CD in water with vortex.

Preparation of Day 2 (25 mg/kg, 10 mL/kg) formulation: 2.5 mg/mL of “10% HP-β-CD in water” solution was prepared by dissolving 39.28 mg of (6aS)-6-methyl-5,6,6a,7-tetrahydro-4H-dibenzo[de,g]quinoline-10,11-diol hydrochloride hemihydrate in 15.260 mL of 10% HP-(3-CD in water with vortex.

Preparation of Day 3 (25 mg/kg, 10 mL/kg) formulation: 2.5 mg/mL of “10% HP-β-CD in water” solution by dissolving 44.36 mg of (6aS)-6-methyl-5,6,6a,7-tetrahydro-4H-dibenzo[de,g]quinoline-10,11-diol hydrochloride hemihydrate in 17.233 mL of 10% HP-β-CD in water with vortex.

Preparation of Day 4 (25 mg/kg, 10 mL/kg) formulation: 2.5 mg/mL of “10% HP-(3-CD in water” solution by dissolving 40.83 mg of (6aS)-6-methyl-5,6,6a,7-tetrahydro-4H-dibenzo[de,g]quinoline-10,11-diol hydrochloride hemihydrate in 15.862 mL of 10% HP-β-CD in water with vortex.

Sample Collection

For Group 1 and 2, blood, CSF and brain samples were collected from 3 mice at the following time points.

| Group | PK Sampling Time Points |
|---|---|
| 1 | 5, 15, 30 min, 1, 2, 3, and 4 hours post dose |
| 2 | 0, 5, 15, 30 min, 1, 2, 3, 4, 8, 12, 24, and |
| | 48 hours post last day (Day 4) dose |

Plasma samples: About 30 μL or 300 μL of blood samples were collected from each animal via dorsal metatarsal vein or heart. These blood samples were placed into the tubes containing K2EDTA. And centrifugation at 3000 g for 10 minutes in a 4° C. centrifuge. The completion time from collection was less than 30 min. The stabilizing agent of ascorbic acid at 200 mg/mL was added in a proportion of 10% in plasma. The plasma samples were stored in a freezer at −75±15° C. prior to analysis. The completion time from collection was less than 60 min.

Brain and CSF Samples: The animals were fully exsanguinated with a rising concentration of CO2 gas prior to CSF and brain collection.

For CSF collection, make an incision by scissors on the neck of the rats, and the foramen magnum is exposed to the sight of scientist. Use a syringe with a venoclysis needle to sample the CSF, the tip of the needle is insert into the membrane of foramen magnum about 1-2 mm, and then pull the stopcock a small length of the syringe to make a negative pressure, then the CSF fluids to the tube. Watch the fluid in the tube and pull the stopcock slowly, when the amount of the CSF is enough, stop and split the needle and the syringe to ensure there is no negative pressure in the tube. Then cut off the needle and collect the CSF fluid. If we could see the blood in the tube, stop immediately, split the needle and the syringe, and then cut off between the blood and CSF to ensure there is no blood in the CSF fluid. Then collect the CSF fluid. The stabilizing agent of ascorbic acid at 200 mg/mL was added in a proportion of 10% in CSF. The completion time from collection was less than 60 min.

For brain collection. Brain samples were collected at adopted time point, and washed 3 times with cold saline, then dried with clean gauze and put into the weighed tube, quick frozen in ice box and kept at −75±15° C. All brain samples were weighted and homogenized with phosphate buffered saline (PBS) by brain weight (g) to PBS volume (mL) ratio 1:3 before analysis. The stabilizing agent of ascorbic acid at 200 mg/mL was added in a proportion of 10% in Brain homogenate. The actual concentration was the detected value multiplied by the dilution factor. Brain samples were collected at designed time point, quickly homogenized and kept at −75±15° C. The completion time from collection was less than 60 min.

Preparation of Standards

About 1 mg (6aS)-6-methyl-5,6,6a,7-tetrahydro-4H-dibenzo[de,g]quinoline-10,11-diol hydrochloride hemihydrate and (6aR)-6-methyl-5,6,6a,7-tetrahydro-4H-dibenzo[de,g]quinoline-10,11-diol hydrochloride hemihydrate was dissolved in DMSO to obtain a 1 mg/mL standard stock solution (free base).

Calibration standard working solutions were prepared at concentrations of 0.2, 0.5, 1, 2, 5, 10, 50, 100, 500, 1000 ng/mL by serial dilution of the standard stock solution. These QC samples were prepared on the day of analysis in the same way as calibration standards.

Sample Treatment

Plasma: The desired serial concentrations of working solutions were achieved by diluting stock solution of analyte with 50% acetonitrile in water solution. 10 μL of working solutions (0.1, 0.2, 0.5, 1, 2, 5, 10, 50, 100, 500, 1000 ng/mL) were added to 10 μL of the blank C57BL/6J Mice plasma to achieve calibration standards of 0.1˜1000 ng/mL (0.1, 0.2, 0.5, 1, 2, 5, 10, 50, 100, 500, 1000 ng/mL) in a total volume of 20 μL. Five quality control samples at 0.2 ng/mL, 1 ng/mL, 2 ng/mL, 50 ng/mL and 800 ng/mL for plasma and brain were prepared independently of those used for the calibration curves. These QC samples were prepared on the day of analysis in the same way as calibration standards. 20 μL of standards, 20 μL of QC samples and 20 μL of unknown samples (10 μL of plasma with 10 μL of blank solution) were added to 200 μL of acetonitrile containing IS mixture for precipitating protein respectively. Then the samples were vortexed for 30 s. After centrifugation at 4 degree Celsius, 4700 rpm for 15 min. The supernatant was diluted 3 times with water. 5 μL of diluted supernatant was injected into the LC/MS/MS system for quantitative analysis.

Brain: The desired serial concentrations of working solutions were achieved by diluting stock solution of analyte with 50% acetonitrile in water solution. 10 μL of working solutions (0.1, 0.2, 0.5, 1, 2, 5, 10, 50, 100, 500, 1000 ng/mL) were added to 10 μL of the blank C57BL/6J Mice brain homogenate to achieve calibration standards of 0.1˜1000 ng/mL (0.1, 0.2, 0.5, 1, 2, 5, 10, 50, 100, 500, 1000 ng/mL) in a total volume of 20 μL. Five quality control samples at 0.2 ng/mL, 0.5 ng/mL, 1 ng/mL, 50 ng/mL and 800 ng/mL for plasma and brain were prepared independently of those used for the calibration curves. These QC samples were prepared on the day of analysis in the same way as calibration standards. 20 μL of standards, 20 μL of QC samples and 20 μL of unknown samples (10 μL of brain homogenate with 10 μL of blank solution) were added to 200 μL of acetonitrile containing IS mixture for precipitating protein respectively. Then the samples were vortexed for 30 s. After centrifugation at 4 degree Celsius, 4700 rpm for 15 min. The supernatant was diluted 3 times with water. 5 μL of diluted supernatant was injected into the LC/MS/MS system for quantitative analysis.

CSF: The desired serial concentrations of working solutions were achieved by diluting stock solution of analyte with 50% acetonitrile in water solution. 10 μL of working solutions (0.1, 0.2, 0.5, 1, 2, 5, 10, 50, 100, 500, 1000 ng/mL) were added to 10 μL of the blank C57BL/6J Mice CSF to achieve calibration standards of 0.1˜1000 ng/mL (0.1, 0.2, 0.5, 1, 2, 5, 10, 50, 100, 500, 1000 ng/mL) in a total volume of 20 μL. Four quality control samples at 0.5 ng/mL, 1 ng/mL, 50 ng/mL and 800 ng/mL for plasma and brain were prepared independently of those used for the calibration curves. These QC samples were prepared on the day of analysis in the same way as calibration standards. 20 μL of standards, 20 μL of QC samples and 20 μL of unknown samples (10 μL of CSF with 10 μL of blank solution) were added to 200 μL of acetonitrile containing IS mixture for precipitating protein respectively. Then the samples were vortexed for 30 s. After centrifugation at 4 degree Celsius, 4700 rpm for 15 min. The supernatant was diluted 3 times with water. 5 μL of diluted supernatant was injected into the LC/MS/MS system for quantitative analysis.

LC-MS/MS Conditions

The LC-MS/MS system consisted of SHIMADZU (LC-30A D, Serial NO. L20555407804 AE and L20555407803 AE; DGU-20A5R Serial NO. L20705413440 IX; CBM-20A Serial NO. L20235428798 CD; SIL-30AC, Serial NO. L20565403657 AE; CTO-30A, Serial NO. L20575401041 CD), Rack Changer II (Serial No. L20585400874 SS), and LCMS-8060 instrument (Serial NO. 011105400164 AE).

Chromatographic separation was performed on a HALO 90A, C18, 2.7 μm, 2.1*50 mm, SN:USAQ001307 at room temperature. The mobile phase was composed of A: Water (5 mM Ammonium Acetate); B: Ethanol (95% methanol in water). The flow rate was 0.6 mL/min. The injection volume was 5 μL.

The calibration line used for sample quantification must consist of at least 6 accepted calibration standards. Acceptance of calibration standards requires calculated concentration within 80%-120% of the nominal concentration. 75% of the calibration standards should be within the acceptable range.

Acceptance Criteria of Quality Control Samples:

At least 3 concentrations of quality control samples (QCs) should be analyzed in a run. Each concentration should include at least 2 individual samples. Acceptance of QCs requires calculated concentration within 80%-120% of the nominal concentration. QCs should be analyzed amongst all unknown samples and ⅔ of the QCs should be within the acceptable range, including at least 1 sample at each concentration level in an analytical run.

Acceptance Criteria of Unknown Samples:

Unknown samples with normal peak shape of analytes and calculated concentration within the calibration range should be accepted. Samples with calculated concentration below LLOQ should be recorded as BLOQ. Samples with calculated concentration above 120% of ULOQ should be diluted with blank matrix and re-assayed. The re-assayed concentration should be multiplied by the dilution factor to obtain the final data. In cases of abnormality, such as equipment malfunction, power outage, sample treatment failure and/or sample injection failure, re-assay should be done in an individual analytical run.

Data acquisition was performed by Lab Solutions version 5.89 Software (Shimadzu). All concentration data was reported with 3 significant figures. Data statistics were performed using Excel 2010 software. The pharmacokinetic parameters of (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol were calculated using a non-compartmental approach with Phoenix™ WinNonlin®.

Results

The pharmacokinetic parameters for (6aS)-6-methyl-5,6,6a,7-tetrahydro-4H-dibenzo[de,g]quinoline-10,11-diol in mice following PO administration were calculated by non-compartmental analysis (Phoenix™ WinNonlin® 8.0), and a linear trapezoidal algorithm was used for AUC calculation.

Plasma pharmacokinetic parameters for (6aS)-6-methyl-5,6,6a,7-tetrahydro-4H-dibenzo[de,g]quinoline-10,11-diol following single dose oral administration to male C57BL/6J Mice at 25 mg/kg:

| PK parameters | Unit | Mean |
|---|---|---|
| T_{1/2} | h | 1.4 |
| Tₘₐₓ | h | 0.1 |
| Cₘₐₓ | ng/mL | 1153 |
| AUCₗₐₛₜ | h* ng/mL | 857 |
| AUC_{Inf} | h* ng/mL | 945 |
| AUC__{%Extrap}_obs | % | 9.34 |
| MRT_{Inf}_obs | h | 1.5 |
| AUCₗₐₛₜ/D | h*mg/mL | 34.3 |
| AUCₗₐₛₜ/Cₘₐₓ | h | 0.743 |

Plasma pharmacokinetic parameters for (6aS)-6-methyl-5,6,6a,7-tetrahydro-4H-dibenzo[de,g]quinoline-10,11-diol following last day (Day 4) repeated dose oral administration to male C57BL/6J Mice at 25 mg/kg:

| PK parameters | Unit | Mean |
|---|---|---|
| T_{1/2} | h | 1.2 |
| Tₘₐₓ | h | 0.1 |
| Cₘₐₓ | ng/mL | 1363 |
| AUCₗₐₛₜ | h*ng/mL | 837 |
| AUC_{Inf} | h*ng/mL | 837 |
| AUC__{%Extrap}_obs | % | 0.0352 |
| MRT_{Inf}_obs | h | 0.9 |
| AUCₗₐₛₜ/D | h*mg/mL | 33.5 |
| AUCₗₐₛₜ/Cₘₐₓ | h | 0.614 |

Brain pharmacokinetic parameters for (6aS)-6-methyl-5,6,6a,7-tetrahydro-4H-dibenzo[de,g]quinoline-10,11-diol following single dose oral administration to male C57BL/6J Mice at 25 mg/kg:

| PK parameters | Unit | Mean |
|---|---|---|
| T_{1/2} | h | 2.9 |
| Tₘₐₓ | h | 0.1 |
| Cₘₐₓ | ng/mL | 729 |
| AUCₗₐₛₜ | h* ng/mL | 215 |
| AUC_{Inf} | h* ng/mL | 264 |
| AUC__{%Extrap}_obs | % | 18.5 |
| MRT_{Inf}_obs | h | 2.05 |
| AUCₗₐₛₜ/D | h*mg/mL | 8.61 |
| AUCₗₐₛₜ/Cₘₐₓ | h | 0.295 |

Brain pharmacokinetic parameters of (6aS)-6-methyl-5,6,6a,7-tetrahydro-4H-dibenzo[de,g]quinoline-10,11-diol following last day (Day 4) repeated dose oral administration to male C57BL/6J Mice at 25 mg/kg:

| PK parameters | Unit | Mean |
|---|---|---|
| T_{1/2} | h | 0.6 |
| Tₘₐₓ | h | 0.1 |
| Cₘₐₓ | ng/mL | 2328 |
| AUCₗₐₛₜ | h* ng/mL | 556 |
| AUC_{Inf} | h* ng/mL | 558 |
| AUC__{%Extrap}_obs | % | 0.393 |
| MRT_{Inf}_obs | h | 0.343 |
| AUCₗₐₛₜ/D | h*mg/mL | 22.3 |
| AUCₗₐₛₜ/Cₘₐₓ | h | 0.239 |

CSF pharmacokinetic parameters for (6aS)-6-methyl-5,6,6a,7-tetrahydro-4H-dibenzo[de,g]quinoline-10,11-diol following single dose oral administration to male C57BL/6J Mice at 25 mg/kg:

| PK parameters | Unit | Mean |
|---|---|---|
| T_{1/2} | h | 2.0 |
| Tₘₐₓ | h | 0.1 |
| Cₘₐₓ | ng/mL | 56.4 |
| AUCₗₐₛₜ | h* ng/mL | 25.5 |
| AUC_{Inf} | h* ng/mL | 35.6 |
| AUC__{%Extrap}_obs | % | 28.4 |
| MRT_{Inf}_obs | h | 2.82 |
| AUCₗₐₛₜ/D | h*mg/mL | 1.02 |
| AUCₗₐₛₜ/Cₘₐₓ | h | 0.452 |

CSF pharmacokinetic parameters of (6aS)-6-methyl-5,6,6a,7-tetrahydro-4H-dibenzo[de,g]quinoline-10,11-diol following last day (Day 4) repeated dose oral administration to male C57BL/6J Mice at 25 mg/kg:

| PK parameters | Unit | Mean |
|---|---|---|
| T_{1/2} | h | 0.2 |
| Tₘₐₓ | h | 0.1 |
| Cₘₐₓ | ng/mL | 200 |
| AUCₗₐₛₜ | h* ng/mL | 38.9 |
| AUC_{Inf} | h* ng/mL | 40.2 |
| AUC__{%Extrap}_obs | % | 3.18 |
| MRT_{Inf}_obs | h | 0.214 |
| AUCₗₐₛₜ/D | h*mg/mL | 1.56 |
| AUCₗₐₛₜ/Cₘₐₓ | h | 0.195 |

(6aS)-6-methyl-5,6,6a,7-tetrahydro-4H-dibenzo[de,g]quinoline-10,11-diol plasma concentrations following single dose oral administration to male C57BL/6J Mice at 25 mg/kg:

| Time | Concentration (ng/mL) | | | Mean | SD | CV |
|---|---|---|---|---|---|---|
| (h) | G1 | G2 | G3 | (ng/mL) | (ng/mL) | (%) |
| 0.0833 | 1120 | 1300 | 1040 | 1153 | 133 | 11.5 |
| 0.25 | 524 | 569 | 658 | 584 | 68 | 11.7 |
| 0.5 | 501 | 701 | 383 | 528 | 161 | 30.4 |
| 1 | 332 | 162 | 210 | 235 | 88 | 37.3 |
| 2 | 100 | 138 | 128 | 122 | 20 | 16.1 |
| 3 | 55.5 | 90.3 | 71.5 | 72.4 | 17.4 | 24.0 |
| 4 | 89.2 | 58.2 | 30.8 | 44.5 | 29.2 | 65.7 |

(6aS)-6-methyl-5,6,6a,7-tetrahydro-4H-dibenzo[de,g]quinoline-10,11-diol plasma concentrations following last day (Day 4) repeated dose oral administration to male C57BL/6J Mice at 25 mg/kg:

| Time | Concentration (ng/mL) | | | Mean | SD | CV |
|---|---|---|---|---|---|---|
| (h) | G1 | G2 | G3 | (ng/mL) | (ng/mL) | (%) |
| 0 | BLOQ | BLOQ | BLOQ | BLOQ | NA | NA |
| 0.0833 | 1460 | 1520 | 1110 | 1363 | 221 | 16.2 |
| 0.25 | 618 | 1280 | 1070 | 989 | 338 | 34.2 |
| 0.5 | 448 | 599 | 313 | 453 | 143 | 31.6 |
| 1 | 112 | 202 | 146 | 153 | 45 | 29.6 |
| 2 | 69.0 | 81.8 | 72.5 | 74.4 | 6.6 | 8.89 |
| 3 | 35.7 | 35.8 | 73.1 | 48.2 | 21.6 | 44.7 |
| 4 | 20.1 | 20.8 | 18.2 | 19.7 | 1.3 | 6.83 |
| 8 | 0.765 | 0.599 | 1.24 | 0.87 | 0.33 | 38.3 |
| 12 | 0.198 | BLOQ | 0.150 | 0.174 | NA | NA |
| 24 | BLOQ | BLOQ | BLOQ | BLOQ | NA | NA |
| 48 | BLOQ | BLOQ | BLOQ | BLOQ | NA | NA |

(6aS)-6-methyl-5,6,6a,7-tetrahydro-4H-dibenzo[de,g]quinoline-10,11-diol brain concentrations following single dose oral administration to male C57BL/6J Mice at 25 mg/kg:

| Time | Concentration (ng/g) | | | Mean | SD | CV |
|---|---|---|---|---|---|---|
| (h) | G1 | G2 | G3 | (ng/g) | (ng/g) | (%) |
| 0.0833 | 636 | 940 | 612 | 729 | 183 | 25.1 |
| 0.25 | 240 | 247 | 208 | 232 | 20.8 | 8.98 |
| 0.5 | 77.2 | 74.0 | 46.0 | 65.7 | 17.2 | 26.1 |
| 1 | 26.7 | 19.0 | 22.0 | 22.6 | 3.85 | 17.1 |
| 2 | 14.9 | 19.0 | 16.5 | 16.8 | 2.10 | 12.5 |
| 3 | 9.68 | 12.4 | 12.4 | 11.5 | 1.58 | 13.7 |
| 4 | 15.4 | 12.6 | 6.84 | 11.6 | 4.38 | 37.7 |

(6aS)-6-methyl-5,6,6a,7-tetrahydro-4H-dibenzo[de,g]quinoline-10,11-diol brain concentrations following last day (Day 4) repeated dose oral administration to male C57BL/6J Mice at 25 mg/kg:

| Time | Concentration (ng/g) | | | Mean | SD | CV |
|---|---|---|---|---|---|---|
| (h) | G1 | G2 | G3 | (ng/g) | (ng/g) | (%) |
| 0 | BLOQ | BLOQ | BLOQ | BLOQ | NA | NA |
| 0.0833 | 3512 | 2388 | 1084 | 2328 | 1215 | 52.2 |
| 0.25 | 756 | 1164 | 592 | 837 | 295 | 35.2 |
| 0.5 | 81.6 | 137 | 51 | 90 | 44 | 48.7 |
| 1 | 56.8 | 27.6 | 24.4 | 36.3 | 17.9 | 49.2 |
| 2 | 40.8 | 13.9 | 10.9 | 21.9 | 16.5 | 75.3 |
| 3 | 4.76 | 5.40 | 10.2 | 6.80 | 3.00 | 44.1 |
| 4 | 2.48 | 2.52 | 2.26 | 2.42 | 0.14 | 5.66 |
| 8 | BLOQ | BLOQ | BLOQ | BLOQ | NA | NA |
| 12 | BLOQ | BLOQ | BLOQ | BLOQ | NA | NA |
| 24 | BLOQ | BLOQ | BLOQ | BLOQ | NA | NA |
| 48 | BLOQ | BLOQ | BLOQ | BLOQ | NA | NA |

(6aS)-6-methyl-5,6,6a,7-tetrahydro-4H-dibenzo[de,g]quinoline-10,11-diol CSF concentrations following single dose oral administration to male C57BL/6J Mice at 25 mg/kg:

| Time | Concentration (ng/mL) | | | Mean | SD | CV |
|---|---|---|---|---|---|---|
| (h) | G1 | G2 | G3 | (ng/mL) | (ng/mL) | (%) |
| 0.0833 | 50.2 | 73.1 | 46.1 | 56.4 | 14.6 | 25.8 |
| 0.25 | 22.7 | 19.8 | 19.0 | 20.5 | 2.0 | 9.53 |
| 0.5 | 9.76 | 6.83 | 6.50 | 7.70 | 1.79 | 23.3 |
| 1 | 3.71 | 3.06 | 3.18 | 3.32 | 0.34 | 10.4 |
| 2 | 3.08 | 4.37 | 3.22 | 3.56 | 0.71 | 19.9 |
| 3 | BLOQ | BLOQ | BLOQ | BLOQ | NA | NA |
| 4 | 4.08 | 3.42 | BLOQ | 3.42 | NA | NA |

(6aS)-6-methyl-5,6,6a,7-tetrahydro-4H-dibenzo[de,g]quinoline-10,11-diol CSF concentrations following last day (Day 4) repeated dose oral administration to male C57BL/6J Mice at 25 mg/kg:

| Time | Concentration (ng/mL) | | | Mean | SD | CV |
|---|---|---|---|---|---|---|
| (h) | G1 | G2 | G3 | (ng/mL) | (ng/mL) | (%) |
| 0 | BLOQ | BLOQ | BLOQ | BLOQ | NA | NA |
| 0.0833 | 344 | 178 | 79.3 | 200 | 134 | 66.9 |
| 0.25 | 41.8 | 67.6 | 41.4 | 50.2 | 15.0 | 29.9 |
| 0.5 | 6.22 | 9.54 | 4.18 | 6.64 | 2.71 | 40.8 |
| 1 | 5.22 | 3.47 | 3.07 | 3.92 | 1.14 | 29.2 |
| 2 | 4.25 | BLOQ | BLOQ | BLOQ | NA | NA |
| 3 | BLOQ | BLOQ | BLOQ | BLOQ | NA | NA |
| 4 | BLOQ | BLOQ | BLOQ | BLOQ | NA | NA |
| 8 | BLOQ | BLOQ | BLOQ | BLOQ | NA | NA |
| 12 | BLOQ | BLOQ | BLOQ | BLOQ | NA | NA |
| 24 | BLOQ | BLOQ | BLOQ | BLOQ | NA | NA |
| 48 | BLOO | BLOO | BLOO | BLOQ | NA | NA |

### Example 9: Plasma Pharmacokinetic Study of (6aS)-6-methyl-5,6,6a,7-tetrahydro-4H-dibenzo[de,g]quinoline-10,11-diol Following Single Dose Oral Administration in Fed Male

Cynomolgus Monkeys

The pharmacokinetic profile of (6aS)-6-methyl-5,6,6a,7-tetrahydro-4H-dibenzo[de,g]quinoline-10,11-diol was evaluated in male Cynomolgus Monkeys. Three male Cynomolgus Monkeys were administrated a single oral dose at 10 mg/kg. The oral vehicle was 10% (w/v) HP-β-CD in water.

Animals

Male Cynomolgus Monkeys were purchased from Topgene Biotechnology. The animals were 2-5 years old with body weights of 2.0-5.0 kg on the dosing date. The animals were housed in a 12-hour light/12-hour dark cycle environment and had free access to food and water.

All animals for SC administration had free access to food and water. All animals for PO administration were fasted overnight prior to dosing and will be fed approximately 2 hours after dosing.

Study Design

Three male Cynomolgus Monkeys were assigned to this study. The animals were treated with (6aS)-6-methyl-5,6,6a,7-tetrahydro-4H-dibenzo[de,g]quinoline-10,11-diol by PO administration at 10 mg/kg, which was formulated in 10% (w/v) HP-β-CD in water. The dosing regimens and formulation (vehicle) compositions are described in the following table below:

| | | Dose Level | Dose Volume | Conc. | Administration | No. of |
|---|---|---|---|---|---|---|
| Group | Treatment | (mg/kg) | (mL/kg) | (mg/mL) | Route | Animals |
| 1 | M102 | 10 | 5 | 2 | PO | 3M |

Dose Administration

Animals were weighed prior to dose administration on the day of dosing for calculating the actual dose volume. The dosages were prepared as illustrated in the table below:

| | | Free | | | Conc | |
|---|---|---|---|---|---|---|
| Compound | Route | MW | MW | FW | (mg/mL) | Vehicle |
| (6aS)-6-methyl- | PO | 267.32 | 303.79 | 312.79 | 2 | 10% |
| 5,6,6a,7- | | | | | | (w/v) |
| tetrahydro-4H- | | | | | | HP-β-CD |
| dibenzo[de,g]quin- | | | | | | in water |
| oline-10,11-diol | | | | | | |

Sample Collection

Blood samples were taken at the following times:

| Group | PK time points |
|---|---|
| PO | Pre-dose, 5 min, 10 min, 15 min, |
| | 30 min, 1, 2, 4, 8 and 24 hr post dose |

Whole blood samples were gently inverted several times to ensure anticoagulation then placed on wet ice prior to centrifugation. Within 30 minutes of collection, the samples were centrifuged at approximately 4,000×g (force) for 5 minutes at 2 to 8° C. to obtain plasma. The stabilizing agent, ascorbic acid at 200 mg/mL, was added in a proportion of 10% in plasma. The plasma samples were stored in a freezer at −75±15° C. prior to analysis. The completion time from collection was less than 60 min. The resultant plasma samples will be immediately separated divided into 2 aliquots (˜75 μL each) and transferred to cryogenic vial. Samples will be maintained on wet ice throughout processing, and stored in a freezer at −75±15° C. prior to analysis. Samples should be protected from ambient light (under sodium lamp).

Preparation of Standards

Standards were prepared with 0.96 mg of (6aS)-6-methyl-5,6,6a,7-tetrahydro-4H-dibenzo[de,g]quinoline-10,11-diol added to 0.820 mL DMSO to obtain a 1 mg/mL standard stock solution (free form).

Calibration standard working solutions were prepared at concentrations of 2, 5, 10, 20, 100, 500, 1000, 5000, 10000 ng/mL by serial dilution of the standard stock solution in 1:1 acetonitrile:water. Quality control working solutions at concentrations of 5, 10, 20, 500 and 8000 ng/mL were prepared by serial dilution of the standard stock solution in 1:1 acetonitrile:water. The QC samples were prepared on the day of analysis in the same way as calibration standards.

Sample Treatment

Samples were prepared with 5 μL of each calibration standard working solution (2, 5, 10, 20, 100, 500, 1000, 5000, 10000 ng/mL) added to 50 μL of the blank Cynomolgus Monkeys plasma to achieve calibration standards concentrations of 0.2, 0.5, 1, 2, 10, 50, 100, 500, 1000 ng/mL (relative to a 50-4, plasma aliquot). Quality Control (QC) samples at 0.5 ng/mL (low-1), 1 ng/mL (low-2), 2 ng/mL (low-3), 50 ng/mL (mid) and 800 ng/mL (high) for plasma were prepared independently for those used for the calibration curves.

Total volumes of 55 μL of standards, 55 μL of QC samples and 55 μL of unknown samples (50 μL of plasma with 5 μL of 1:1 acetonitrile:water) were added to 200 μL of acetonitrile containing IS to precipitate proteins. Then the samples were vortexed for 30 sec. After centrifugation at 4° C., 3900 rpm for 15 min, supernatant was diluted 5 times with water, and 2 μL of the diluted supernatant was injected into the LC-MS/MS system for quantitative analysis. All of the samples were processed on ice.

LC-MS/MS Conditions

The LC-MS/MS system consisted of two Shimadzu LC-30AD pumps, a DGU-20A5R degasser, a Rack changer II and an AB Sciex Triple Quad 5500 LC/MS/MS mass spectrometer. Chromatographic separation was performed on Agilent ZORBAX XDB-Phenyl 5 μm (50×2.1 mm) at room temperature. The mobile phase was composed of A: 5% Acetonitrile/95% water (0.1% Formic acid); B: 95% Acetonitrile/5% water (0.1% Formic acid). The flow rate was 0.6 mL/min. The injection volume was 2 μL.

Positive mode electrospray ionization (ESI) was performed on a Turbo V® ion source to obtain a protonated ion of (6aS)-6-methyl-5,6,6a,7-tetrahydro-4H-dibenzo[de,g]quinoline-10,11-diol and Dexamethasone (IS). A multiple reaction monitoring (MRM) method was selected for quantitative analysis. The optimized transitions were 268.05/237.10 Da and 393.14/373.10 Da for (6aS)-6-methyl-5,6,6a,7-tetrahydro-4H-dibenzo[de,g]quinoline-10,11-diol and Dexamethasone, respectively. The instrument parameters were set as follows: ion spray voltage: 5500 V; curtain gas: 40 psi; ion source gas 1: 50 psi; ion source gas 2: 50 psi; temperature: 450° C. The compound dependent parameters are listed as follows:

| | (6aS)-6-methyl-5,6,6a,7- | |
|---|---|---|
| | tetrahydro-4H- | |
| | dibenzo[de,g]quinoline- | Dexamethasone |
| | 10,11-diol | (IS) |
| Precursor/ | 268.05/237.10 | 393.14/373.10 |
| product | | |
| DP | 131.00 | 146.00 |
| CE | 25.00 | 13.00 |
| CXP | 18.00 | 16.00 |

HPLC flow rate was set at 0.6 mL/min with the following conditions:

| Time (min) | A (%) | B (%) |
|---|---|---|
| 0.01 | 95.0 | 5.00 |
| 0.20 | 95.0 | 5.00 |
| 1.90 | 5.00 | 95.0 |
| 2.20 | 5.00 | 95.0 |
| 2.21 | 95.0 | 5.00 |
| 2.50 | 95.0 | 5.00 |

Results

Following the PO (11.4 mg/kg) t_{1/2} was 2.26±0.56 hr, Tₘₐₓ was 0.667±0.289 hr, Cₘₐₓ was 34.7±17.9 ng/mL, MRT_{Inf} was 2.92±0.92 hr, AUC₀₋₂₄ hr was 58.1±12.4 h*ng/mL, and AUC_{inf} was 63.5±11.9 h*ng/mL in Cynomolgus Monkeys.

The following table illustrates the plasma pharmacokinetic parameters of (6aS)-6-methyl-5,6,6a,7-tetrahydro-4H-dibenzo[de,g]quinoline-10,11-diol following PO administration to male Cynomolgus Monkeys at 10 mg/kg:

| PK parameters | Unit | 168777C | 169939C | 168779C | Mean | SD | CV(%) |
|---|---|---|---|---|---|---|---|
| T_{1/2} | h | 5.60 | NA | NA | NA | NA | NA |
| Tmax | h | 2.00 | 4.00 | 4.00 | 3.33 | 1.15 | 34.6 |
| Cmax | ng/mL | 6.27 | 5.91 | 8.04 | 6.74 | 1.14 | 16.9 |
| AUCₗₐₛₜ | h*ng/mL | 42.6 | 33.9 | 47.9 | 41.5 | 7.1 | 17.1 |
| AUC_{Inf} | h*ng/mL | 44.9 | NA | NA | NA | NA | NA |
| AUC_% Extrap_obs | % | 5.01 | NA | NA | NA | NA | NA |
| MRTInf_obs | h | 6.52 | NA | NA | NA | NA | NA |
| AUCₗₐₛₜ/D | h*mg/mL | 4.26 | 3.39 | 4.79 | 4.15 | 0.71 | 17.1 |
| F | % | NA | NA | NA | NA | NA | NA |
| AUCₗₐₛₜ/Cₘₐₓ | h | 6.79 | 5.74 | 5.96 | 6.16 | 0.56 | 9.1 |

(6aS)-6-methyl-5,6,6a,7-tetrahydro-4H-dibenzo[de,g]quinoline-10,11-diol Plasma Concentrations Following PO Administration to Male Cynomolgus Monkeys at 10 mg/kg in fed condition:

| Time | Concentration (ng/mL) | | | Mean | SD | CV |
|---|---|---|---|---|---|---|
| (h) | 168777C | 169939C | 168779C | (ng/mL) | (ng/mL) | (%) |
| 0 | BLOQ | BLOQ | BLOQ | NA | NA | NA |
| 0.083 | 4.62 | 1.07 | 0.425 | 2.04 | 2.26 | 111 |
| 0.167 | 2.83 | 1.23 | 0.911 | 1.66 | 1.03 | 62.1 |
| 0.25 | 2.63 | 0.893 | 0.262 | 1.26 | 1.23 | 97.2 |
| 0.5 | 4.15 | 0.975 | 1.26 | 2.13 | 1.76 | 82.5 |
| 1 | 5.15 | 2.81 | 5.00 | 4.32 | 1.31 | 30.3 |
| 2 | 6.27 | 5.41 | 6.27 | 5.98 | 0.50 | 8.30 |
| 4 | 4.40 | 5.91 | 8.04 | 6.12 | 1.83 | 29.9 |
| 8 | 1.13 | 2.61 | 0.779 | 1.51 | 0.97 | 64.5 |
| 24 | 0.278 | BLOQ | 0.279 | 0.279 | NA | NA |

The present invention is not to be limited in scope by the specific embodiments described herein. Indeed various modifications of the invention in addition to those described herein will be apparent to those skilled in the art from the foregoing description and the accompanying Figures. Such modifications are intended to fall within the scope of the appended claims.

It is further to be understood that all values are approximate and are provided for description. All references cited and discussed in this specification are incorporated herein by reference in their entirety and to the same extent as if each reference was individually incorporated by reference.

1. A method of treating a neurodegenerative disease, the method comprising:
administering a pharmaceutical composition comprising (6aS)-6-methyl-5,6,6a,7-tetrahydro-4H-dibenzo[de,g]quinoline-10,11-diol and at least one pharmaceutically acceptable excipient,
wherein the composition provides a pharmacokinetic profile comprising a t_{1/2} at about 0.25 to about 4 hours after oral administration to a mammal.

2. (canceled)

3. (canceled)

4. A method of treating a neurodegenerative disease, the method comprising:
administering a pharmaceutical composition comprising (6aS)-6-methyl-5,6,6a,7-tetrahydro-4H-dibenzo[de,g]quinoline-10,11-diol and at least one pharmaceutically acceptable excipient,
wherein the composition provides a pharmacokinetic profile comprising a t_{1/2} at about 0.5 to about 7 hours after intramuscular, subcutaneous, sublingual, buccal, or intranasal administration.

5. (canceled)

6. (canceled)

7. A method of treating a neurodegenerative disease, the method comprising:
administering a pharmaceutical composition comprising (6aS)-6-methyl-5,6,6a,7-tetrahydro-4H-dibenzo[de,g]quinoline-10,11-diol and at least one pharmaceutically acceptable excipient,
wherein the composition provides a t_{1/2} at about 0.05 to about 5 hours after intravenous administration.

8. (canceled)

9. (canceled)

10. The method of claim 1, wherein the composition provides a pharmacokinetic profile comprising a Tₘₐₓ at about 0.25 to about 10 hours after oral administration.

11. The method of claim 4, wherein the composition provides a pharmacokinetic profile comprising a Tₘₐₓ at about 0.05 to about 10 hours after intramuscular, subcutaneous, sublingual, buccal, or intranasal administration.

12. (canceled)

13. (canceled)

14. (canceled)

15. (canceled)

16. (canceled)

17. (canceled)

18. The method of claim 4, wherein the composition provides a pharmacokinetic profile comprising a Cₘₐₓ at about 1 ng/mL to about 5000 ng/mL after intramuscular, subcutaneous, sublingual, buccal, or intranasal administration.

19. (canceled)

20. (canceled)

21. The method of claim 1, wherein the composition provides a pharmacokinetic profile comprising a Cₘₐₓ at about 10 ng/mL to about 2000 ng/mL after oral administration.

22. (canceled)

23. The method of claim 1, wherein the composition provides a brain to plasma ratio in a mammal for (6aS)-6-methyl-5,6,6a,7-tetrahydro-4H-dibenzo[de,g]quinoline-10,11-diol of about 0.1 to about 6 following administration.

24. The method of claim 1, wherein the composition provides a CSF to plasma ratio in a mammal for (6aS)-6-methyl-5,6,6a,7-tetrahydro-4H-dibenzo[de,g]quinoline-10,11-diol of about 0.1 to about 6 following administration.

25. (canceled)

26. The method of claim 1, wherein the composition provides an oral bioavailability of about 2% to about 30% relative to intravenous administration.

27. (canceled)

28. The method of claim 4, wherein the composition provides an intramuscular or subcutaneous bioavailability of about 40% to about 99% relative to intravenous administration.

29. The method of claim 4, wherein the composition provides a sublingual or buccal bioavailability of about 2% to about 80% relative to intravenous administration.

30. The method of claim 4, wherein the composition provides an intranasal bioavailability of about 20% to about 80% relative to intravenous administration.

31. (canceled)

32. (canceled)

33. The method of claim 1, wherein the composition provides an oral bioavailability of about 2% to about 30% relative to intramuscular or subcutaneous administration.

34. (canceled)

35. (canceled)

36. (canceled)

37. (canceled)

38. (canceled)

39. (canceled)

40. The method of claim 4, wherein the composition provides a provides an AUCₗₐₛₜ/Cₘₐₓ ratio from about 0.01 to about 20 hours after intramuscular or subcutaneous administration.

41. (canceled)

42. The method of claim 4, wherein the composition provides an AUCₗₐₛₜ/Cₘₐₓ ratio from about 0.01 to about 20 hours after sublingual or buccal administration.

43. (canceled)

44. The method of claim 4, wherein the composition provides an AUCₗₐₛₜ/Cₘₐₓ ratio from about 0.01 to about 20 hours after intranasal administration.

45. (canceled)

46. The method of claim 1, wherein the composition provides an AUCₗₐₛₜ/Cₘₐₓ ratio from about 0.01 to about 20 hours after oral administration.

47. (canceled)

48. The method of claim 7, wherein the composition provides an AUCₗₐₛₜ/C₀ ratio from about 0.0001 to about 20 hours after intravenous administration.

49. (canceled)
